(19) United States
(12) Patent Application Publication
(10) Pub. No.: US 2026/0234159 A1
(43) Pub. Date: Aug. 13, 2026

(54) SMALL MOLECULE TREATMENT OF FATTY LIVER DISEASE AND HCC

(71) Applicant: SCANDIEDGE THERAPEUTICS AB, Stockholm (SE)

(72) Inventors: Adil MARDINOGLU, Göteborg (SE); Mathias UHLÉN, Lidingö (SE); Jan BORÉN, Göteborg (SE); Jihad SEBHAOUI, Ümraniye/Istanbul (TR); Sajda ASHRAF, Ümraniye/Istanbul (TR); Shazia IQBAL, Ümraniye/Istanbul (TR)

(21) Appl. No.: 19/156,173

(22) PCT Filed: Feb. 15, 2024
(86) PCT No.: PCT/EP2024/053887

§ 371 Date: Aug. 13, 2025

(30) Foreign Application Priority Data
Feb. 15, 2023 (EP) 23156937.7

(51) Int. Cl. C07D 495/04; A61K 31/381; A61K 31/4045; A61K 31/416; A61K 31/4192; A61K 31/4365; A61K 31/4436; A61K 31/4439; A61K 31/444; A61K 31/4535; A61K 31/4709; A61K 31/506; A61K 31/5377; A61P 1/16; A61P 35/00; C07D 333/68; C07D 333/80; C07D 409/12; C07D 409/14

(52) U.S. Cl. CPC C07D 495/04; A61K 31/381; A61K 31/4045; A61K 31/416; A61K 31/4192; A61K 31/4365; A61K 31/4436; A61K 31/4439; A61K 31/444; A61K 31/4535; A61K 31/4709; A61K 31/506; A61K 31/5377; A61P 1/16; A61P 35/00; C07D 333/68; C07D 333/80; C07D 409/12; C07D 409/14

(57) ABSTRACT

There is provided a compound of formula (I) or a pharmaceutically acceptable salt thereof.

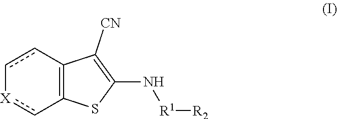

### TECHNICAL FIELD

The present disclosure relates to a pharmaceutical compound used for the treatment of fatty liver disease and hepatocellular carcinoma.

### BACKGROUND

Non-alcoholic fatty liver disease (NAFLD) is defined as the accumulation of fat in the liver due to the imbalance in the uptake and secretion of fat as well as the increased de novo lipogenesis (DNL) or decreased oxidation of fat in the liver [1]. NAFLD can progress to non-alcoholic steatohepatitis (NASH). It has been reported that the prevalence of NAFLD is approximately 25% of the population, but it has no approved effective pharmacological treatment. Hence, there is an urgent need for the development of an effective treatment strategy.

Previously, the research groups including the present inventors have generated gene co-expression networks (CNs) for the liver and 45 other primary human tissues, and identified the pyruvate kinase L/R (PKLR) as a target, whose inhibition may selectively inhibit DNL in the liver [2]. The PKLR gene encodes for the liver (PKL) and erythrocyte (PKR) isoforms of the enzyme and catalyses the production of pyruvate and ATP from phosphoenolpyruvate and ADP. PKL and PKR isoforms are specifically expressed in the liver and erythrocytes, respectively. An independent mouse population study has also verified the driving role of PKLR in the development of NAFLD [3]. Recently, in vitro experiments were performed by inhibiting and overexpressing PKLR in HepG2 cells, and it was found that the expression of PKLR was significantly positively correlated with the expression of FASN, de novo lipogenesis (DNL), triacylglycerol (TAG) levels and cell growth [4].

Furthermore, the small molecule, JNK-IN-5A, has shown potential in suppressing the expression levels of PKLR and may thereby be used in the treatment of NAFLD [5].

### SUMMARY

The present inventors have realized that there is a possibility to develop and optimize small molecules, which may be used in the treatment of fatty liver disease and hepatocellular carcinoma (HCC). The development/optimization was carried out by altering the molecular structure of JNK-IN-5A to improve the effect of the small molecules in a treatment.

According to a first aspect of the present disclosure, there is provided a compound of formula (I)

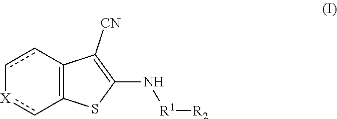

or a pharmaceutically acceptable salt thereof, wherein:
both [symbol] are singles bond or double bonds;
X is —(CH₂)₂— or —NR₃— when [symbol] is a single bond;
X is —CH— when [symbol] is a double bond;
R1 is independently selected from a carbonyl and a sulphonyl;
R2 is selected from the group consisting of

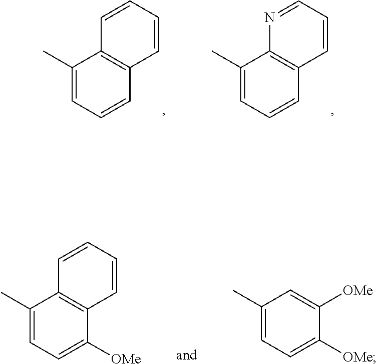

R3 is selected from the group consisting of —H,

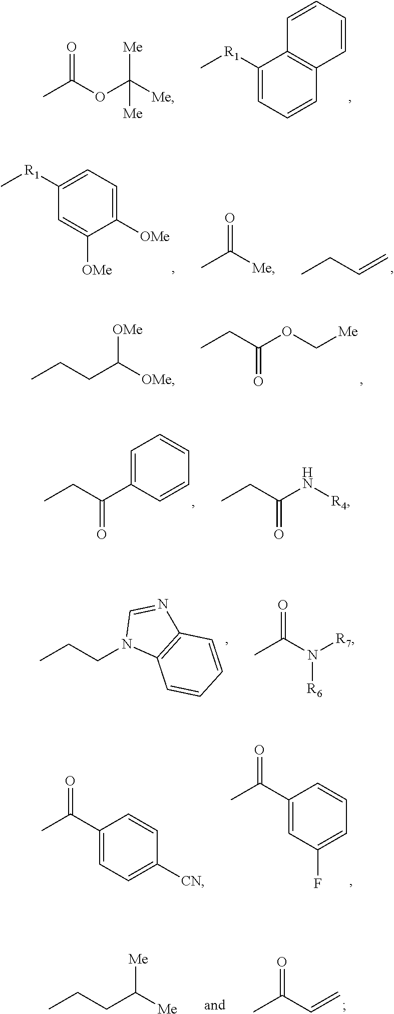

R4 is

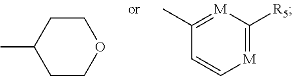

M is N or C;
R5 is —H, a halide,

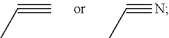

R6 is —H, -Me or phenyl; and
R7 is C1-C3 alkyl or an optionally substituted aryl, provided that it is not a compound according to any one of formulae (II)-(V):

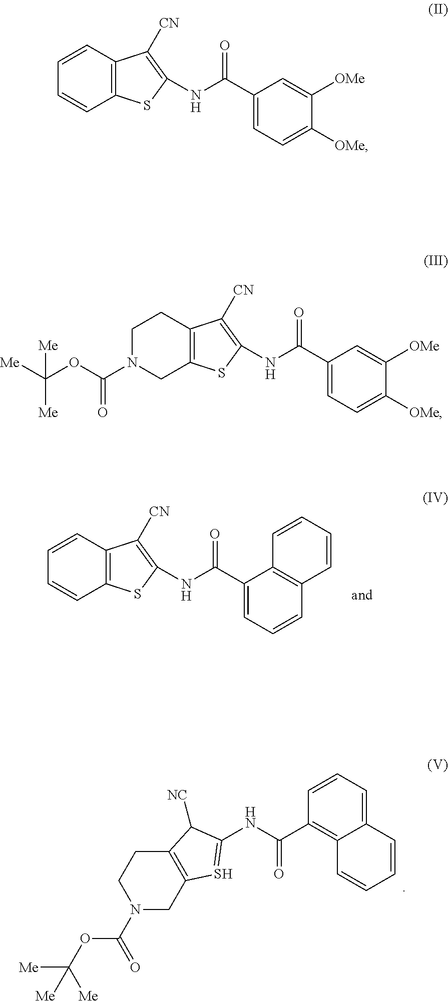

JKN-IN-5A has previously been identified as a drug for use as in the treatment of fatty liver disease, especially non-alcoholic fatty liver disease (NAFLD), due to the molecule's ability to suppress the expression levels of PKLR and lower the triglyceride (TAG) levels. The present inventors have realized that the molecular structure of JNK-IN-5A can be tuned in order to optimize the effect it has on the expression levels of PKLR, TAG levels and cell viability. Molecules according to formula (I) were proved to be active and treatment with these molecules reduces the TAG levels and/or cell viability making them suitable for use in the treatment of NAFLD and/or HCC.

In one embodiment, X is —(CH₂)₂— or —NR₃—, and [symbol] is a single bond.

In one embodiment, R4 is selected from the group consisting of:

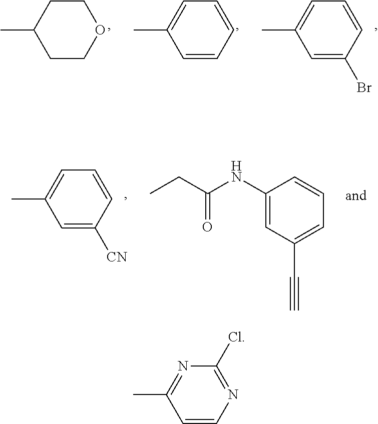

Compounds B130, B164 and B170-172 in the Examples section below are included in this embodiment and shown to be particularly active. Preferably, R4 is selected from

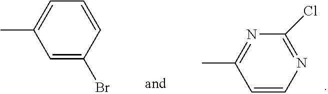

Compound B171 and B172 in the Examples section below are included in this preferred embodiment and treatment with these compounds results in a particularly low cell viability of HepG2 cells especially in a cancerous media.

In one embodiment, R3 is selected from the group consisting of

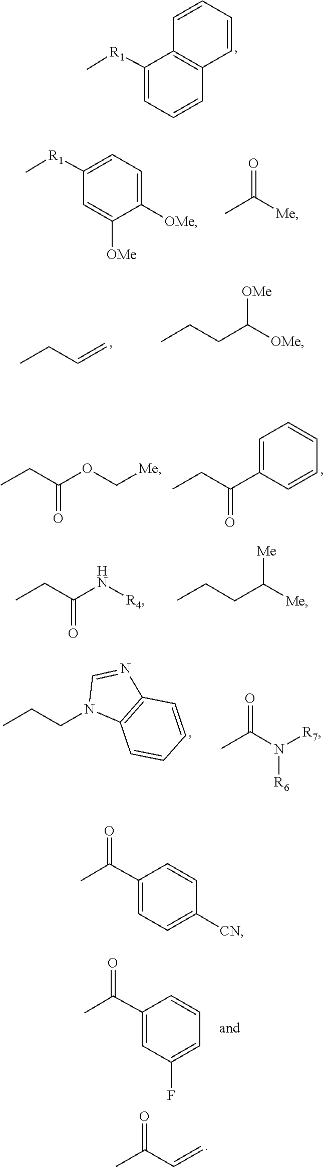

Preferably, R3 is selected form the group consisting of:

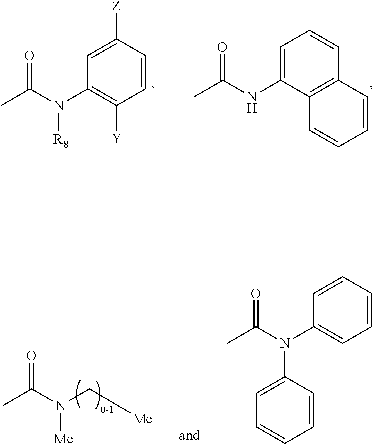

wherein:
Z is selected from the group consisting of —H, -Me and —OMe;
Y is selected from the group consisting of —H, —F, —Br, —Cl, —OMe, —OCF₃, —CF₃, -Me and —NO₂; and
R8 is selected from —H and -Me.

Compounds A117, A119, A151-A154 and A156-A164 in the Examples section below are included in this embodiment and shown to result in particularly low TAG levels.

Compounds A156, A158 and A159 result in exceptionally low cell viability and may hence be particularly suitable for treatment of HCC. Accordingly, it may be preferred that Y is —Cl, —OCF₃ or —CF₃.

Another compound of particular interest in HCC treatment is B171 shown in the Examples section below.

In one embodiment, Z is selected from the group consisting of —H, -Me and —OMe; Y is selected from the group consisting of —H, —F, —Br, —Cl, —OMe, -Me and —NO₂; and R8 is selected from —H and -Me. This embodiment may be associated with lower toxicity.

In further embodiments:
R1 is a carbonyl;
[symbol] is a single bond and X is —NR₃—; and/or
R2 is

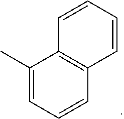

These structural features are found in the most effective compounds of the Examples section below.

The compound may have the formula (VI)

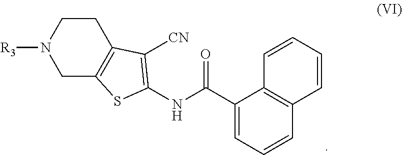

In a preferred embodiment, R3 in formula (VIII) is selected from the group consisting of

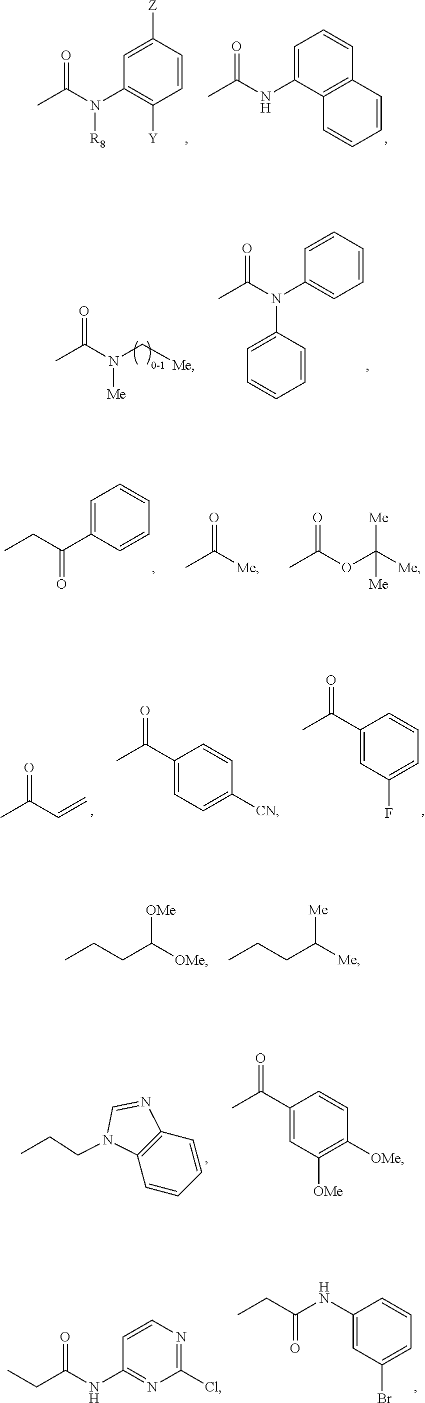

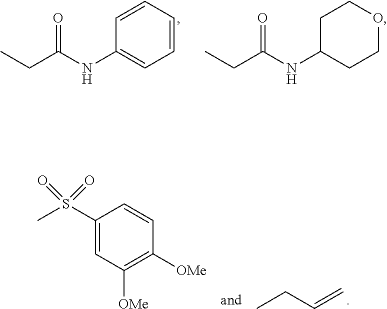

In this embodiment, one of the following applies:
Z is —H, Y is —F, —Br, —Cl, —OMe or —NO₂, and R8 is —H;
Z is —OMe, Y is —H, and R8 is —H;
Z is -Me, Y is —F, and R8 is —H; or
Z is —H, Y is —H, and R8 is -Me.

Compounds A114, A119, A117, A131-A132, A151-A154, A156-A157, A160-A164, A179, A190, A192-A193, B129-B130, B133, B153, B164 and B173 in the Examples section below are included in this embodiment and exhibit a particularly good ratio between TAG content and cell viability which is suitable for the treatment of fatty liver disease.

More preferably, R3 in formula (VIII) is selected from

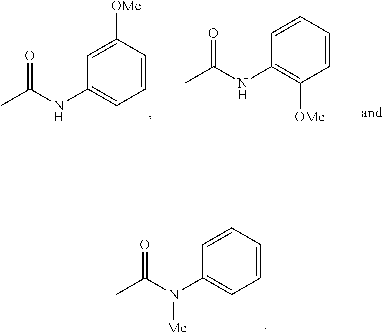

These compounds, A151, A152 and A162 in the Examples section, exhibited a low ratio between TAG content and cell viability making them particularly interesting for treatment of fatty liver disease.

In another embodiment, X is —(CH₂)₂— and R2 is preferably

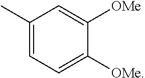

Compound A135 in the Examples section belongs to this embodiment. Treatment with these compounds results in a significantly low cell viability of HepG2 cells especially in cancerous media, making them particularly suitable for use in the treatment of HCC.

The compound according to the present invention may be used as a medicament.

According to a second aspect, a pharmaceutical composition is provided comprising a compound according to the first aspect.

According to a third aspect, a compound according to formulae (V) or (VII)

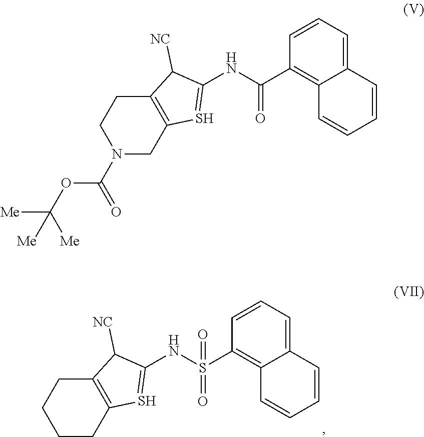

or a compound according to the first aspect or a pharmaceutical composition according to the second aspect is provided for use in a method of treatment of fatty liver disease or hepatocellular carcinoma (HCC).

In one embodiment of the third aspect, the method of treatment comprises oral administration of the compound or pharmaceutical composition.

The method of treatment may further comprise detection of overexpression of PKLR in the liver of a subject to be treated prior to administration of the compound to the subject. Overexpression may for example be a higher level of expression than a reference level corresponding to an average level of expression in healthy liver tissue. Alternatively, overexpression may be a higher level of expression than a reference level corresponding to an average level of expression in NAFLD or HCC liver tissue.

The subject of the treatment of the third aspect is preferably a human.

As a fourth aspect of the present disclosure, there is provided a method of treatment of fatty liver disease or HCC in a subject in need thereof, said method comprising administering to the subject a therapeutically effective amount of a compound of formula (I).

The embodiments and examples of the first to third aspect apply to the fourth aspect mutatis mutandis.

### BRIEF DESCRIPTION OF THE DRAWINGS

FIG. 1 shows a schematic view of the synthesis of N-(3-cyano-4,5,6,7-tetrahydrobenzo[b]thiophen-2-yl)-1-naphthamide analogues.

FIG. 2 shows a schematic view of the synthesis of N-(3-cyano-4,5,6,7-tetrahydrobenzo[b]thiophen-2-yl)naphthalene-1-sulfonamide analogues.

FIG. 3 shows a schematic view of the synthesis of triazole analogues of N-(3-cyano-4,5,6,7-tetrahydrobenzo[b]thiophen-2-yl)-1-naphthamide.

FIG. 4 shows a schematic view of the synthesis of triazole analogues of N-(3-cyano-4,5,6,7-tetrahydrobenzo[b]thiophen-2-yl)naphthalene-1-sulfonamide.

FIG. 5 shows a schematic view of the synthesis of 2-amino-4,5,6,7-tetrahydrobenzo[b]thiophene-3-carbonitrile analogues.

FIG. 6 shows a schematic view of the synthesis of amide analogues of 2-amino-5,6,7,8-tetrahydro-4H-cyclohepta[b]thiophene-3-carbonitrile.

FIG. 7 shows a schematic view of the synthesis of amide analogues of 2-aminobenzo[b]thiophene-3-carbonitrile.

FIG. 8 shows a schematic view of the synthesis of analogues of aminothiophene.

FIG. 9 shows a schematic view of the synthesis of tert-butyl 2-amino-3-cyano-4,7-dihydrothieno[2,3-c]pyridine-6(5H)-carboxylate analogues.

FIG. 10 shows a schematic view of the synthesis of an analogue of N-(3-cyano-4,5,6,7-tetrahydrobenzo[b]thiophen-2-yl)-1-naphthamide with a linker PKL-B52.

FIG. 11 shows a schematic view of the synthesis of N-(3-cyano-4,5,6,7-tetrahydrothieno[2,3-c]pyridin-2-yl)-1-naphthamide.

FIG. 12 shows a schematic view of the synthesis of Analogues of N-(3-cyano-4,5,6,7-tetrahydrothieno[2,3-c]pyridin-2-yl)-1-naphthamide.

FIG. 13 shows a list of the synthesized urea analogues of N-(3-cyano-4,5,6,7-tetrahydrothieno[2,3-c]pyridin-2-yl)-1-naphthamide obtained via isocyanates.

FIG. 14 shows a list of the synthesized urea analogues urea analogues of N-(3-cyano-4,5,6,7-tetrahydrothieno[2,3-c]pyridin-2-yl)-1-naphthamide obtained via carbamoyl chlorides.

FIG. 15 shows a list of the synthesized amide and sulfonamide analogues of N-(3-cyano-4,5,6,7-tetrahydrothieno[2,3-c]pyridin-2-yl)-1-naphthamide.

FIG. 16 shows a list of the synthesized alkyl analogues of N-(3-cyano-4,5,6,7-tetrahydrothieno[2,3-c]pyridin-2-yl)-1-naphthamide.

FIG. 17 shows a list of the synthesized heterocyclic ring containing analogues of N-(3-cyano-4,5,6,7-tetrahydrothieno[2,3-c]pyridin-2-yl)-1-naphthamide.

FIG. 18 shows the scoring of histopathological and immunohistochemical findings in rats liver tissues.

FIG. 19-24 show western blotting of PKLR after 4 days of 10 μM drug treatment. JNK and JNK-5A are abbreviations for JNK-IN-5A in these figures.

FIG. 25 shows screening of potential drug candidates. Western blotting of PKLR after 2 respectively 4 days of 10 μM drug treatment. JNK is an abbreviation for JNK-IN-5A in this figure.

FIG. 26-34 shows western blotting of PKLR and steatosis related pathway proteins with one week steatosis model. JNK is an abbreviation for JNK-IN-5A in this figure.

FIG. 35-41 show the triglyceride level either in percent or O.D at 570 nm after one week steatosis model. JNK and JNK-5A are abbreviations for JNK-IN-5A in these figures.

FIG. 42-47 show the cell viability after 2 days incubation with 10 μM drug treatment. JNK and JNK-5A are abbreviations for JNK-IN-5A in these figures.

FIG. 48 shows the cell viability after 4 days incubation with 10 μM drug treatment. JNK is an abbreviation for JNK-IN-5A in this figure.

FIG. 49 shows the cell viability after 1 week steatosis with 10 μM drug treatment. JNK is an abbreviation for JNK-IN-5A in this figure.

FIG. 50 shows TAG contents and cell viability after 1 week steatosis with 10 μM drug treatment. JNK is an abbreviation for JNK-IN-5A in this figure. TAG contents are represented by the grey staples, and the cell viabilities are represented by the black staples.

FIG. 51 shows the ratio between TAG contents and cell viability. JNK is an abbreviation for JNK-IN-5A in this figure.

FIG. 52 shows a schematic view of the JNK activity test using ab273417.

FIG. 53 shows the JNK activity using purified JNK1, JNK2 and JNK3.

FIG. 54-55 shows the protein-ligand binding results.

FIG. 56 shows the ranking of tested target compounds based on cell viability.

FIG. 57 shows the ranking of tested target compounds based on TAG contents.

FIG. 58 shows the ranking of tested target compounds based on the ratio between TAG contents and cell viability.

FIG. 59 shows the cell viability (MTT) of HepG2 cells after treatment with JNK-IN-5A derivatives for one week steatosis, four days and two days at 10 μM. * P<0.05.

FIG. 60 shows that JNK-IN-5A, its derivatives, sorafenib and regorafenib induce apoptosis, cell cycle arrest, and autophagy via p53 pathway. (A-B) Time dependent cell viability assay (MTT) for day 1 to day 4 at 10 μM drug concentration. * P<0.05.

FIG. 61 shows the DAPI and PI staining for necrotic cell death analysis. HepG2 cells were treated with 10 μM of the JNK-IN-5A derivatives for 2 days and cells were stained with DAPI (Blue) and PI (Red) for 30 min, at live cells. PI positive necrotic cells were counted and plotted.

FIG. 62 shows the IC50 values for hit compounds after treatment for 2 days. The cell viability at 8 different concentrations—30 μM, 10 μM, 3 μM, 1 μM, 300 nM, 100 nM, 30 nM and untreated was observed. * P<0.05.

FIG. 63 shows the whole lysate western blot analysis of cells treated with JNK-IN-5A, its derivatives, sorafenib and regorafenib.

FIG. 64 shows the nuclear protein western blot analysis of cells treated with (A) Control, sorafenib, SET156 and SET158; (B) JNK-IN-5A, regorafenib, SET159, SET171, SET135 and SET172.

FIG. 65 shows the FACs analysis with PI staining cell treated with (A) JNK-IN-5A, its derivatives, sorafenib and regorafenib.

FIG. 66 shows the mitochondrial membrane potential assay of cells treated with JNK-IN-5A, its derivatives, sorafenib and regorafenib. 2 μM TMRE staining HepG2 cells treated with 10 μM compound 1 day. * P<0.05.

FIG. 67 shows the autophagy assay of cells treated with JNK-IN-5A, its derivatives, sorafenib and regorafenib. Autophagic vacuoles were stained with GFP fluorescence. GFP fluorescence signals were measured by region of interest (ROI) using ImageJ program. * P<0.05.

FIG. 68 shows the time dependent LDH assay. Day 1 to day 4 of treating cells with JNK-IN-5A, its derivatives, sorafenib and regorafenib.

FIG. 69 shows the western blot analysis of HepG2 cells treated with JNK-IN-5A, its derivatives, sorafenib and regorafenib. Cellular expression level of p62 and p-S349 p62.

FIG. 70 show the cellular ROS assay. 20 μM DCFDA staining on HepG2 cells treated with 10 μM of JNK-IN-5A, its derivatives, sorafenib and regorafenib for 1 day.

FIG. 71 shows the lipid peroxidation assay for HepG2 cells treated with JNK-IN-5A, its derivatives, sorafenib and regorafenib. Ratiometric lipid peroxidation sensor stained peroxidised lipid. Fluorescence intensity were measured on region of interest (ROI) using Image. * P<0.05, scale bar=100 μm.

FIG. 72 shows the invasion assay. HepG2 cells were treated with 1 μM of JNK-IN-5A, its derivatives, sorafenib and regorafenib and invasion assay was observed after 24 hr. * P<0.05.

FIG. 73 shows the wound healing assay. Cancer cell motility were measured for 4 days on HepG2 cells treated with 1 μM of JNK-IN-5A, its derivatives, sorafenib and regorafenib. * P<0.05.

FIG. 74 show a PCA plot showing the distribution of HepG2 cells treated by JNK-IN 5A, its derivatives, sorafenib and regorafenib.

FIG. 75 shows the number of differentially expressed genes under different treatments.

FIG. 76 shows the GSOA of downregulated genes induced by the treatment SET135 and SET171.

FIG. 77 shows GSOA based on significantly up regulated genes for each treatment vs. control in HepG2 cells.

FIG. 78 shows GSOA based on significantly downregulated genes for each treatment vs. control in HepG2 cells.

FIG. 79 shows (A) the Jaccard index between DEGs from different treatments and (B) GO semantic similarity between significant GO terms from different treatments.

FIG. 80 shows the GSEA of representative hallmark gene sets in different treatments compared to control NES (normalized enrichment score).

FIG. 81 shows the GSEA of representative KEGG pathways in different treatments compared to control NES, normalized enrichment score.

FIG. 82 shows the activity scores (presented as z score) of the 14 cancer related PROGENy pathways for the nine treatments in Example 2.

FIG. 83 shows the 2D-interactions of six compounds with active site crucial residues of JNK-3.

FIG. 84 shows that PKL silencing with siRNA did not induce cytotoxicity. (A) Cell viability assay (MTT) of HepG2 cells with siRNA silenced PKL. (B) Western blot analysis of HepG2 cells with siRNA silenced PKL.

FIG. 85 shows the prediction of the most potential mechanism of action (the shortest path (A) from target compounds (JNK-IN-5A derivatives); (B) Regorafenib; and (C) Sorafenib to protein TP53 or MYC. Notably, JNK IN 5A and the 6 target compounds all have the same predicted pathways. Confidence scores calculated by Open MoA for all edges in the predicted MoA are greater than 0.99.

### DETAILED DESCRIPTION

JNK-IN-5A has been showed to work as a drug for the treatment of fatty liver disease. However, the molecular structure needs to be tuned in order to optimize the reduction of the expression levels of PKLR and other steatosis related proteins as well as the reduction of triglyceride levels.

NAFLD is a chronic liver disease characterized by the accumulation of fat in the liver. The prevalence of NAFLD has been increasing rapidly and emerging as a prominent health concern worldwide due to its association with obesity, metabolic syndrome and diabetes [6]. However, no medical treatment has been approved for the treatment of NAFLD, yet. Therefore, more targeted and effective drugs are needed for NAFLD treatment. Pyruvate kinase is a key enzyme in glycolysis and the PKLR gene encodes PKL and PKR isoforms of pyruvate kinase in the liver and erythrocytes, respectively, and they are expressed at low levels in most other tissues where their roles are replaced by PKM [4, 5, 7]. This indicates that PKLR could be an effective target for a treatment strategy for NAFLD with minimal side effects on other human tissues. In the present disclosure, PKLR and JNK-5A were regarded as target gene and reference drug for NAFLD and HCC treatment.

Molecular docking studies have been performed (see Methods—Molecular docking) and based on these, a list of small molecules was synthesized. The activity, TAG levels and cell viability of the small molecules was determined.

Accordingly, a compound according to the first aspect is provided and treatment with these compounds is shown to provide an improved effect in the reduction of TAG levels compared to JNK-IN-5A. The compound according to the first aspect or a composition according to the second aspect may be used in the treatment of fatty liver disease or HCC. The fatty liver disease may be non-alcoholic fatty liver disease (NAFLD) or may have progressed to non-alcoholic steatohepatitis (NASH).

Thus, treatment with a compound according to the first aspect or a pharmaceutical composition according to the second aspect is shown to reduce the PKL expression, TAG levels and the expression of steatosis-related proteins (Acetyl-CoA carboxylase, fatty acid synthase 1 and Carbohydrate response element binding protein) in a steatosis cell model. It has further been shown in this disclosure that treatment with a compound according to the first aspect reduced the PKL expression while having no adverse effects on PKM expression. Thus, these compounds may be regarded as PKL-specific drug candidates for the treatment of NAFLD with minimum side effects on other tissues.

In recent years, researchers have started to uncover a potential relationship between NAFLD and the development of HCC. The evidence suggests that advanced stages of NAFLD, particularly NASH and fibrosis, could contribute to an increased risk of developing HCC [8]. HCC is still one of the deadliest cancer types with a low survival rate and typically exhibits a lower response rate to chemotherapy than many other cancers [9-11]. Current clinical treatments often include multi-target kinase inhibitors such as sorafenib and regorafenib. However, the need for novel therapeutic options is evident due to modest efficacy of the existing therapies [54] and the development of chemoresistance in some patients [55,56].

The PKLR gene has previously been reported as a target for HCC treatment and JNK-IN-5A which is a potent inhibitor of PKLR protein expression, has been suggested as a new therapeutic agent for treatment of HCC [2, 5].

Thus, a compound according to the first aspect or a pharmaceutical composition according to the second aspect is provided for use in a method of treatment of HCC.

In addition to decreasing PKL expression, treatment with compounds according to the first aspect have demonstrated anticancer effects via the p53 signalling pathway. Treatment with these compounds induces the expression of p53-dependent tumour suppressor proteins, decreases c-Myc levels, and leads to a significant increase in p21 expression. Furthermore, these compounds' have the ability to selectively enhance nuclear p21 expression which provides strong evidence of their capacity to induce G2/M phase cell cycle arrest. Compounds according to the first aspect, especially A135 and B171, have shown remarkable anticancer effects, inducing both apoptotic and necrotic cell death in HepG2 cells. Moreover, these compounds significantly augment nuclear p53 expression and reduce c-Myc expression, surpassing the effects of JNK-IN-5A, sorafenib, and regorafenib.

### EXAMPLES

### Example 1

### Methods

### Rat Animal Studies

In vivo biosafety studies. Healthy Sprague Dawley male rats (12 weeks old, 250-280 g) were obtained from Atatürk University Experimental Research Center (ATADEM, Erzurum Turkey) and housed under standard environmental conditions (temperature 20° C.-25° C., humidity 50±20% and 12-hour light/dark diurnal cycle). Food and water were available ad libitum. Rats were randomly separated into one control group and two experimental groups (30 mg/kg/day BX-912 or JNK-IN-5A, n=5). BX-912 and JNK-IN-5A were dissolved in DMSO and orally administrated using oral gavage for 7 days. The control group were received the same amount of vehicle solution only.

After 7 days, rats were anaesthetized, and blood samples were collected from the abdominal aorta for biochemical and haematological analysis. The plasma was separated and stored at −80° C. for further analysis. Major organs such as heart, kidney, liver, muscle, adipose, intestine (duodenum, ileum, jejunum), pancreas, colon, and stomach were collected from all the animals for further analysis. Selected organs were fixed in 10% neutral buffered formalin for histopathological examination.

In addition to sampling for biochemical and haematological analysis, one drop of blood samples was obtained from the animals for evaluating micronucleus frequencies as genotoxicity endpoint at the end of the experiments. Three different smears of each rat were prepared using pre-coded and cold slides, then the slides air-dried at room temperature, and fixed in ethanol for 10 min, and stained with Giemsa. The frequencies of micronucleated polychromatic erythrocytes (MNPCEs) were analysed according to previously suggested approach using a light microscope. The incidence of MN-PCEs was determined by scoring a total of 3000 PCE per animal and results were expressed as % MN [12].

In vivo efficacy studies. After 1-week acclimation period, rats were randomly separated into two groups: control group (n=5) and high sucrose group (n=20). The control group was fed with a standard diet while the others consumed high sucrose diet (HSD). Sucrose was purchased from Sigma Chemical Co. (St Louis, MO, USA) and supplied at the dose of 10% in the drink water for two weeks.

After 2 weeks, five rats in the HSD group were randomly sacrificed for conformation of NAFLD model. Then, the remaining rats in the HSD group were separated into three sub-groups (n=5): HSD group, HSD plus BX-912 group and HSD plus JNK-IN-5A group. BX-912 and JNK-IN-5A were dissolved in DMSO and were orally administrated using oral gavage for one week (30 mg/kg/day). The HSD group were treated with vehicle solution only.

At the end of the experiment, rats were sacrificed after being anesthetized. Blood samples were collected from the abdominal aorta and centrifuged at 8000 rpm for 15 min at 4° C. The plasma was separated and stored at −80° C. until further analysis. Other internal organs, including the heart, adipose tissues, liver, kidney, muscle, intestine (duodenum, ileum, jejunum), pancreas, colon and stomach were immediately removed and then snap frozen in liquid nitrogen and stored at −80° C.; the liver was also fixed in 10% formaldehyde for histopathological examination.

The biosafety and efficacy studies were approved by The Ethics Committee of Atatürk University, and all experiments were carried out in accordance with relevant guidelines and regulations for the care and use of laboratory animals. Animal Experiments Local Ethics Committee of Atatürk University approved the experimental procedure described in this study (Approval Date: 27 Aug. 2020; Approval Number: 42190979-000-E.2000208344).

Histopathological examination. The liver tissues were fixed in 10% buffered formaldehyde solution. After fixation, the tissues were passed through graded alcohol and xylene series and embedded in paraffin blocks. 5 micrometer thick sections were taken serially from the paraffin blocks. Histopathological changes were evaluated by performing hematoxylin eosin staining on the sections taken. Sections were evaluated according to histopathological findings as none (−), mild (+), moderate (++) and severe (+++).

Immunohistochemical examination. After deparaffinization, the slides were immersed in antigen retrieval solution (pH 6.0) and heated in microwave for 15 min to unmask the antigens. The sections were then dipped in 3% H₂O₂ for 10 min to block endogenous peroxidase activity. Protein block was dropped onto the sections, washed with PBS for 10 min. Primary antibodies (8-OH-dG, Cat No: sc-66036, Diluent Ratio: 1/100, Santa Cruz) were prepared and applied according to the usage conditions. Expose mouse and rabbit specific HRP/DAB detection IHC kit were used as follows: sections were incubated with goat anti-mouse antibody, then with streptavidin peroxidase, and finally with 3,3′ diamino benzidine+chromogen. Slides were counter stained with hematoxylin. Immunoreactivity in the sections were graded as none (−), mild (+), moderate (++) and severe (+++).

### Cell Culture Experiments and In Vitro Steatosis Induction

HepG2 wild type cells were purchased from genome engineering company Synthego. Cells were maintained with Roswell Park Memorial Institute growth media, RPMI, 1640 (R2405, Sigma-Aldrich) and supplemented with 10% fetal bovine serum (FBS, F7524, Sigma-Aldrich), 1% penicillin-streptomycin (P/S, P4333, Sigma-Aldrich). Steatosis induction media was prepared as high glucose Dulbecco's Modified Eagle Media (DMEM, D0819, Sigma-Aldrich) with 10% FBS, 1% P/S media supplemented with 10 μg/ml insulin (I9278, Sigma-Aldrich), and 10 μM T0901317 (T2320, Sigma-Aldrich). HepG2 cells were seeded 6×104 cells per well for 96-well plate and 1×106 cells per well for 6-well plate. HepG2 cells were incubated with steatosis induce media (SM) for one week by 3 day+2 day+2 day with total three times media exchange.

### Triglyceride (TAG) Measurement Assay

HepG2 cells were seeded at 6×104 cells per well into 96 well plate and induced steatosis for one week with 10 μM of target drugs. After steatosis induction, cells were washed with 200 μl of phosphate buffered saline (PBS) and triglyceride contents was measured by Triglyceride Assay Kit—Quantification (ab65336, Abcam) following manufacturer's instruction. Optical density (O.D) values were detected with microplate reader at 570 nm (Hidex Sense Beta Plus).

### MTT Assay Measurements

The cytotoxic effects of the synthesized drugs on the HepG2 cells were tested by MTT (Thiazolyl Blue Tetrazolium Bromide) method. HepG2 cells were seeded 2×104 cells per well in 96-well plate in 200 μL growth media. After 2 days of cell seeding, 10 μM of the respective target drugs were dissolved in 0.1% DMSO and 200 μL of the media were treated for two days. After the incubation period, 5 mg/ml MTT (M2128, Sigma-Aldrich) solution in PBS (10 μL) was added to each well. After 1 hr, the MTT solution and all media were removed from the wells. 100 μl of DMSO was added and mixed to dissolve the formazan crystals. Cell viability was analysed by measuring the absorbance of the dissolved formazan in a microplate reader at a wavelength of 570 nm with microplate reader (Hidex Sense Beta Plus).

### Western Blot

To test the effect of the synthesized drugs on the expression levels of PKLR and steatosis-related proteins, HepG2 cells were seeded 2.5×105 and 5×105 per well in 6-well plate, respectively. After 4 and 7 days of 10 μM drug treatments for PKLR and steatosis assays, respectively, the cells were washed with PBS solution and then lysed with CelLytic M (C2978, Sigma-Aldrich) lysis buffer containing protease inhibitors (11836170001, Roche). The cell lysates were centrifuged at 12.000 rpm for 10 min and supernatants were collected. Protein Assay Dye Reagent (5000006, Bio-Rad) was used for the determination of protein concentration. The absorbance of the proteins was measured spectrophotometrically at 595 nm by a microplate reader (Hidex Sense Beta Plus). Protein electrophoresis was performed using Mini-PROTEAN® TGX™ Precast Gels (4561086, Bio-Rad) and then the separated proteins were transferred to a Trans-Blot Turbo Mini 0.2 μm PVDF Transfer Packs membrane (1704158, Bio-Rad) by using Trans-Blot® Turbo™ Transfer System (Bio-Rad). The membrane was blocked with 5% skim milk for 30 min at 4° C. with gentle rocking. After blocking, the membrane was treated with primary antibodies: Anti-PKLR (HPA006653, Atlas Antibodies, Sigma-Aldrich), Anti-PKM2 (D78A4, Cell Signalling Tech.) Anti-Chrebp (ab92809, Abcam), Anti-Acetyl-CoA Carboxylase (NBP2-55439, Novus Biologicals), Anti-Fatty Acid Synthase (ab22759, Abcam) and Anti-Beta actin (ab8227, Abcam) as a control. The membrane was treated at 4° C. overnight on a rocking platform. After the treatment of primary antibodies, the membrane was washed three times with mixture of tris-buffered saline and Polysorbate 20 (TBS-T buffer, A09-7500-100, Medicago). The membrane was then treated with Goat anti-Rabbit IgG-HRP (ab205718, Abeam) as the secondary antibody. The treatment was carried out at 4° C. for 30 min with gentle rocking. The protein bands on the membrane were revealed using enhanced chemiluminescence substrate (WBLUF0500, Merck) and detected with ImageQuant™ LAS 500 (GE Healthcare).

### JNK Enzyme Activity Assay

JNK activity was measured with JNK Activity Assay Kit (ab273417, Abcam). JNK activity assay was performed with purified JNK, JNK1 (PV3319, Thermo Fisher), JNK2 (PV3620, Thermo Fisher) JNK3(PV4563, Thermo Fisher). 0.3 μg of each JNK purified protein were incubated with c-Jun Protein/ATP in Kinase Assay Buffer for 1 hr followed by manufacturer's instruction with 10 μM small molecules. Catalysed substrate Phospho-c Jun (Ser 73) was detected by western blot analysis. Western blot analysis performed with JNK Activity Assay kit supplied anti-Phospho-cJun (Ser 73) Specific Antibody at 1:10,000 for overnight and Goat Anti-Rabbit HRP (ab205718, abeam) at 1:10,000 for 1 hr incubation as primary and secondary antibody.

### Cellular Thermal Shift Assay (CETSA)

HepG2 cells were trypsinized and calculated at 1×10⁶ cells into 1.5 ml micro centrifuge tube with 100 μl cell culture media. Cells were incubated in a CO₂ incubator at 37° C. for 1 hr with 10 μM of each target drug. Drug treated cells were heated at 60° C. for 3 min using a heat plate. Heated cells were lysed with Native lysis buffer (ab156035, Abcam). Thermal shifted JNK proteins were detected with western blot analysis using JNK1+2+3 antibody (Ab179461, Abcam) and band intensity was analysed with ImageJ program.

### Ranking of Target Molecules

HepG2 cells were seeded at 60,000 cells per well in 96 well plate. Using steatosis induce media, the cells were treated with 10 μM of target drugs for one week. The media was changed 3 times at day 0, day 3 and day 5. TAG assay was performed using MAK266-1KT SDS Triglyceride Quantification Colorimetric kit and cell viability analysis was performed using MTT assay.

### Molecular Docking

Computational docking studies were performed using the MOE v. 2019.01 [13]. The protein structure with the PDB ID code: 202U was prepared using the protein preparation module in MOE by the addition of missing atoms and residues, correction of bond order and formal charge and adjustment of tautomer [14]. System was protonated using the protonate 3D algorithm and generalized Born volume integral (GB/VI) was used as the electrostatics function, with a value of 80 as dielectric constant. The electrostatics and van der Waals cut off were set as 10 and 15 Å, respectively. Protein was charged and minimized with the application of AMBER10:EHT force field implemented in a MOE software.

All of the compounds were built using ChemBioDraw Ultra 14.0, charged and minimized by MMFF94× force field, along with the adjustment of hydrogens and lone pairs [15]. The dataset was subjected to energy minimization using default RMS gradient of 0.1 kcal/mol/Å2. Following protonation and minimization, the compounds were saved in the MOE database format.

The active site was defined to include all atoms within 5 Å of cognate ligand. Before execution of docking, benchmarking of different combination of scoring and placement methods in MOE Dock was performed to find the most suitable combination of algorithm and scoring functions for our target protein. In this study, induced fit docking procedure along with Triangle Matcher algorithm as placement method and LondonDG as initial scoring and GBVI/WSA dG as re-scoring method presented the most reliable results. For each compound, 10 individual docking runs were conducted and 100 conformations were generated. The best-ranked solution of each compound was further assessed for protein-ligand binding analysis.

### Synthesis Procedure of Target Compounds

### General Synthesis Procedure

All reactions were performed using oven-dried glassware and under an inert atmosphere (nitrogen) unless otherwise stated. All reagents and dry solvents were obtained from the commercial supplier Sigma-Aldrich and used without further purification. Organic solutions were concentrated under reduced pressure on a Heidolph rotary evaporator. Reactions were monitored by liquid chromatography-mass spectrometry (LC-MS, Thermo Fisher TSQ Series, Athena C18-WP100 Å, 2.1×50 mm, 3 μm); Water:MeOH (0.01 formic acid)) or by Thin-Layer Chromatography (TLC) using silica gel pre-coated aluminium plates (Kieselgel 60, 254, E. Merck, Germany). The chromatograms were visualized using ultraviolet light (254 and 366 nm) and stained with vanillin dips or aqueous potassium permanganate solution. ¹H NMR spectra were recorded on Advance Bruker 500 MHz spectrometer in CDCl₃ and DMSO-d6. ¹³C NMR spectra were recorded in deuterated solvents on Bruker spectrometer at 126 MHz, with the central peak of the deuterated solvent as the internal standard. The ¹H NMR spectra are reported as δ/ppm downfield from tetramethyl silane (multiplicity, number of protons, coupling constant J/Hz). The ¹³C NMR spectra are reported as δ/ppm. All chemical shifts are reported in parts per million (ppm) relative to the residual solvent peak. The following abbreviations are used to denote signal patterns: (s) singlet, (d) doublet, (t) triplet, (q) quartet, (m) multiplet, and (br) broad unless otherwise stated. Flash-column chromatography was performed on PuriFlash XS 520 Plus Flash chromatography system (Interchim) with built-in UV-detector, ELSD-detector and fraction collector with Interchim silica gel columns.

### Amines and Acyl Chlorides Reactions (General Procedure A)

To a solution of the selected amine in acetonitrile (0.1 M), triethylamine (1.2 equiv.) and the selected acylchloride (1 to 1.2 equiv.) was added. The reaction mixture was stirred at room temperature for 1 h, concentrated, mounted on silica and purified by flash chromatography to afford the desired amides.

### Amide Coupling (General Procedure B)

To a solution of carboxylic acid (1 equiv.), 1-Ethyl-3-(3-dimethylaminopropyl)carbodiimide (EDCl, 1.2 equiv.) and HOBt (1.2 equiv.) in DMF (0.1-0.3 M) and triethylamine or DIPEA (2 equiv.) were added at 0° C. The mixture was stirred for 10 min at room temperature. Then the desired amine (1.2 equiv.) was added, and the reaction mixture was stirred at room temperature for 6-16 h. The residue was poured onto ice and then extracted with ethyl acetate. The combined organic extracts were washed with brine, dried over magnesium sulphate and evaporated to dryness under reduced pressure, mounted on silica and purified by flash chromatography to afford the desired product.

### Ester Hydrolysis (General Procedure C)

To a solution of the selected ethyl ester in water/THF (1:1, 0.1 M), LiOH (2-3 equiv.) was added. The reaction mixture was stirred at room temperature for 1 h. Reaction mixtures were concentrated to approximately half their volume on a rotavap, acidified to pH 1-2 by the addition of HCl (aq., 1 M), and filtered to afford the desired carboxylic acids. Compounds were dissolved, mounted on silica and purified by flash chromatography to afford the desired products.

### Alkylation of Secondary Amines (General Procedure D)

A solution of secondary amine (1 equiv.), potassium carbonate (1.2 equiv.) and TBAB (0.01 equiv.) in THF or DMF was stirred for 30 min at room temperature. Then Alkyl Bromide (2-3 equiv.) was added, and the reaction mixture was stirred at room temperature for 48 h. The residue was partitioned between water and ethyl acetate and the aqueous phase was extracted with further ethyl acetate. The combined organic extracts were washed with brine, dried over magnesium sulphate and evaporated to dryness under reduced pressure to afford the desired product.

### Azide Preparation (General Procedure E)

A solution of alkyl bromide (1 equiv.) and sodium azide (2 equiv.) in EtOH/H2O (2:1) was stirred at room temperature for 2 h. The mixture was evaporated under reduced pressure then extracted with diethyl ether and water. The combined organic extracts were dried over magnesium sulphate and evaporated to dryness under reduced pressure to give the desired product.

### Copper(I)-Catalyzed Alkyne-Azide Cycloaddition (CuAAC) Reactions (General Procedure F)

To a solution of alkyne (1 equiv.) and copper sulphate (0.75 equiv.) in dichloromethane (DCM)/H₂O (2:1), sodium ascorbate (0.25 equiv.) was added. The mixture was stirred at room temperature for 20 min. Then appropriate azide (1.5 equiv.) was added and reaction was stirred for further 6-16 h. The mixture was then extracted with DCM and water. The combined organic extracts were dried over magnesium sulphate and evaporated to dryness under reduced pressure to yield the desired products. Compounds were purified by flash chromatography.

### Boc Group Deprotection (General Procedure G)

To a solution of protected amine in DCM, trifluoroacetic acid (TFA) (DCM:TFA 4:1) was added in the presence of nitrogen. The mixture was stirred at room temperature for 1 hour. The reaction progress and results were confirmed by TLC and LC/MS. When the reaction was completed, after concentration under reduced pressure, ether or ethanol was added and crystallized to obtain the target compounds.

### Synthesis of Target Compounds

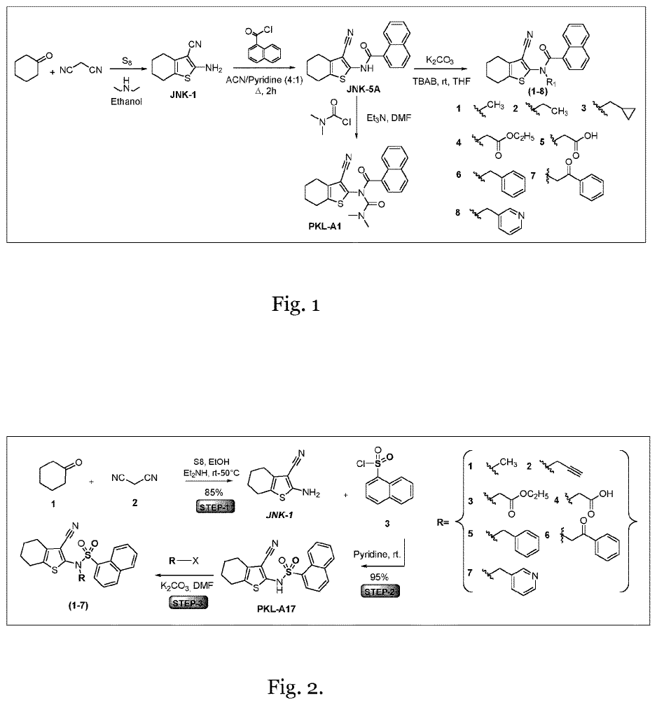

A schematic view of the synthesis of N-(3-cyano-4,5,6,7-tetrahydrobenzo[b]thiophen-2-yl)-1-naphthamide analogues can be seen in FIG. 1 and the synthesis procedure for the specific compounds follows below.

### Synthesis of 2-amino-4,5,6,7-tetrahydrobenzo[b]thiophene-3-carbonitrile (H(

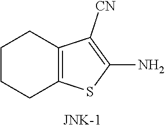 [SMILES: N#Cc1c(N)sc2c1CCCC2]

The compound was synthesized according to the literature procedure [4]. To a stirred solution of cyclohexanone (10.4 ml, 0.10 mol) in ethanol (300 ml) were added sulphur (3.52 g, 0.11 mol), malononitrile (6.60 g, 0.10 mol) and diethylamine (10.30 ml, 0.10 mol). The reaction mixture was heated at 50° C. for 3 hours. The mixture was allowed to cool to room temperature and the solvent was removed under reduced pressure. The residue was partitioned between water and ethyl acetate and the aqueous phase was extracted with additional ethyl acetate. The combined organic extracts were washed with brine, dried over magnesium sulphate and evaporated to dryness under reduced pressure to give the desired product (16.7 g, 94%) as a brown solid.

### Synthesis of N-(3-cyano-4,5,6,7-tetrahydrobenzo[b]thiophen-2-yl)-1-naphthamide (JNK-5A)

A solution of (aminothiophene, JNK-1) (1 g, 5.61 mmol) in ACN (5 ml) with 1-naphthoyl chloride (1 mL, 6.73 mmol) was stirred together at 80° C. under an inert atmosphere for 2 h. The reaction was monitored by TLC and LCMS/MS. The mixture was then evaporated to dryness and chromatographed over silica gel using a gradient of hexane/EtOAc (9:1 to 8:2). The product fractions were pooled and evaporated to afford the title compound.

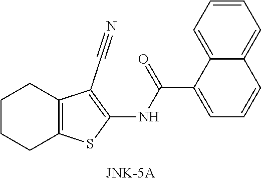 [SMILES: N#Cc1c(NC(=O)c2cccc3ccccc23)sc2c1CCCC2]

### Synthesis of N-(3-cyano-4,5,6,7-tetrahydrobenzo[b]thiophen-2-yl)-N-(prop-2-yn-1-yl)-1-naphthamide (PKL-A65)

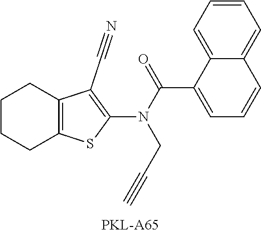 [SMILES: C#CCN(C(=O)c1cccc2ccccc12)c1sc2c(c1C#N)CCCC2]

Following general procedure D, JNK-5A (500 mg, 1.5 mmol), was reacted with propargyl bromide solution (0.013 ml, 1.8 mmol), to afford the title compound as a yellow liquid after column chromatography (EtOAc in Hexane: 0-10%).

¹H NMR (500 MHz, CDCl3) δ 7.98 (d, J=8.4 Hz, 1H), 7.75 (d, J=7.2 Hz, 3H), 7.48 (dt, J=34.2, 7.3 Hz, 3H), 7.32 (s, 1H), 4.72 (s, 0H), 2.42 (s, 4H), 2.33 (s, 1H), 1.65 (s, 4H). ¹³C NMR (126 MHz, CDCl₃) δ 135.16, 133.23, 132.31, 129.26, 127.20, 126.29, 125.57, 124.99, 124.75, 124.14, 124.05, 123.54, 112.27, 73.22, 66.95, 28.67, 23.50, 23.13, 21.62, 20.66, 18.71.

### Synthesis of Ethyl 2-(N-(3-cyano-4,5,6,7-tetrahydrobenzo[b]thiophen-2-yl)-1-naphthamido)acetate (PKL-A3)

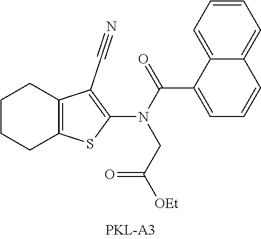 [SMILES: CCOC(=O)CN(C(=O)c1cccc2ccccc12)c1sc2c(c1C#N)CCCC2]

Following general procedure D, JNK-5A (100 mg, 0.30 mmol) reacted with ethyl bromoacetate solution (0.040 ml, 0.36 mmol) to afford 90 mg (71%) of the title compound as a transparent liquid after column chromatography (EtOAc in Hexane: 0-20%).

¹H NMR (500 MHz, CDCl₃) δ 8.12 (d, J=8.5 Hz, 1H), 7.76-7.69 (m, 2H), 7.52 (t, J=7.7 Hz, 1H), 7.44 (dt, J=15.1, 7.3 Hz, 2H), 7.29 (t, J=7.8 Hz, 1H), 4.63 (d, J=6.2 Hz, 2H), 4.26 (q, J=7.3 Hz, 2H), 2.34 (d, J=35.6 Hz, 3H), 1.30 (t, J=7.2 Hz, 2H). 13C NMR (126 MHz, CDCl₃) δ 168.32, 150.31, 136.38, 133.84, 133.26, 132.33, 130.28, 130.16, 128.04, 127.23, 126.54, 125.63, 124.91, 124.50, 113.22, 108.87, 68.22, 61.91, 61.07, 52.11, 24.37, 24.06, 22.59, 21.66, 14.20.

### Synthesis of N-benzyl-N-(3-cyano-4,5,6,7-tetrahydrobenzo[b]thiophen-2-yl)-1-naphthamide (PKL-A4)

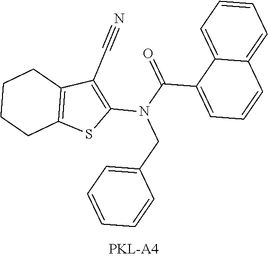 [SMILES: N#Cc1c(N(Cc2ccccc2)C(=O)c2cccc3ccccc23)sc2c1CCCC2]

Following general procedure D, JNK-5A (100 mg, 0.30 mmol) reacted with benzyl bromide (0.35 ml, 0.36 mmol) to afford 116 mg (91%) of the title compound as a transparent liquid after column chromatography (EtOAc in Hexane: 0-20%).

¹H NMR (500 MHz, CDCl₃) δ 8.00 (d, J=8.5 Hz, 1H), 7.79-7.69 (m, 2H), 7.55-7.39 (m, 4H), 7.32-7.22 (m, 5H), 5.17 (s, 2H), 2.32 (s, 4H), 1.59 (s, 4H). ¹³C NMR (126 MHz, CDCl3) δ 135.48, 133.96, 133.37, 132.89, 130.05, 128.81, 128.71, 128.30, 128.24, 128.15, 127.22, 126.52, 125.23, 125.06, 124.55, 29.71, 24.39, 24.09, 22.63, 21.67.

### Synthesis of 2-(N-(3-cyano-4,5,6,7-tetrahydrobenzo[b]thiophen-2-yl)-1-naphthamido)acetic acid (PKL-A2)

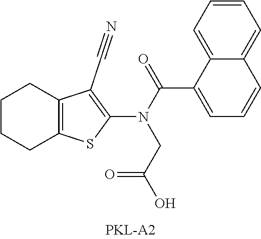 [SMILES: N#Cc1c(N(CC(=O)O)C(=O)c2cccc3ccccc23)sc2c1CCCC2]

Following general procedure C, JNK-5A (350 mg, 0.83 mmol reacted with lithium hydroxide (70.19 mg, 1.67 mmol) to afford 255 mg (78%) of the title compound as a yellow solid after column chromatography (EtOAc in Hexane: 0-20%).

¹H NMR (500 MHz, CDCl₃) δ 8.08 (d, J=8.4 Hz, 1H), 7.79-7.69 (m, 3H), 7.47 (ddd, J=27.0, 15.1, 7.2 Hz, 5H), 7.34-7.25 (m, 2H), 2.38 (s, 2H), 2.32 (s, 2H), 1.60 (s, 4H). ¹³C NMR (126 MHz, CDCl₃) δ 176.25, 172.48, 135.50, 132.93, 132.23, 131.00, 129.27, 127.04, 126.27, 125.55, 124.49, 124.04, 123.45, 50.89, 28.68, 23.36, 23.03, 21.55, 20.62, 19.73.

### Synthesis of N-(3-cyano-4,5,6,7-tetrahydrobenzo[b]thiophen-2-yl)-N-(dimethylcarbamoyl)-1-naphthamide (PKL-A1)

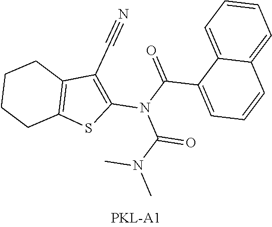 [SMILES: CN(C)C(=O)N(C(=O)c1cccc2ccccc12)c1sc2c(c1C#N)CCCC2]

Following general procedure A, JNK-5A (100 mg, 0.30 mmol) reacted with N-methycarbamoyl chloride (29.4 ul, 0.30 mmol) to afford 90 mg (74%) of the title compound as yellow crystals after column chromatography (EtOAc in Hexane: 0-20%).

¹H NMR (500 MHz, CDCl₃) δ 9.15 (d, J=8.7 Hz, 1H), 7.90 (d, J=8.1 Hz, 1H), 7.84-7.75 (m, 2H), 7.62 (dd, J=8.6, 6.9 Hz, 1H), 7.44 (dt, J=29.1, 7.6 Hz, 2H), 3.25 (s, 3H), 2.89 (s, 3H), 2.65 (dt, J=16.3, 5.6 Hz, 5H), 1.80 (q, J=7.5 Hz, 4H). ¹³C NMR (126 MHz, CDCl₃) δ 151.30, 150.33, 149-50, 136.08, 134.16, 134-10, 132.87, 130.96, 129.10, 128.57, 128.54, 128.08, 126.56, 126.35, 124.51, 114.71, 109.77, 37.07, 36.88, 25.01, 24.18, 23.07, 22.12.

### Synthesis of N-(3-cyano-4,5,6,7-tetrahydrobenzo[b]thiophen-2-yl)-N-(2-oxo-2-phenylethyl)-1-naphthamide (PKL-B26)

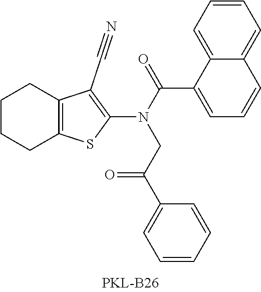 [SMILES: N#Cc1c(N(CC(=O)c2ccccc2)C(=O)c2cccc3ccccc23)sc2c1CCCC2]

Following general procedure D, JNK-5A (100 mg, 0.30 mmol), reacted with 2-Bromoacetophenone (0.35 ml, 0.36 mmol) to afford 85 mg (63%) of the title compound as a liquid after column chromatography (EtOAc in Hexane: 0-20%).

¹H NMR (500 MHz, CDCl₃) δ 8.27 (d, J=8.4 Hz, 1H), 7.98 (d, J=7.7 Hz, 2H), 7.71 (dd, J=8.2, 4.0 Hz, 2H), 7.60-7.50 (m, 3H), 7.44 (dt, J=8.1, 4.1 Hz, 3H), 7.29 (dd, J=8.2, 7.1 Hz, 1H), 5.38 (s, 2H), 2.35 (s, 3H), 2.26 (s, 3H), 1.55 (p, J=3.2 Hz, 4H). ¹³C NMR (126 MHz, CDCl₃) δ 191.36, 170.11, 149.85, 135.59, 133.56, 133.08, 132.52, 132.25, 131.62, 129.35, 129.00, 127.89, 127.14, 126.93, 126.26, 125.50, 124.88, 123.85, 123.47, 112.47, 107.71, 55.99, 23.28, 22.98, 21.66, 21.63, 21.52, 20.63.

### Synthesis of N-(3-cyano-4,5,6,7-tetrahydrobenzo[b]thiophen-2-yl)-N-(2-oxo-2-phenylethyl)-1-naphthamide (PKL-B29)

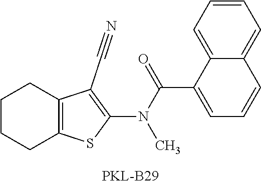 [SMILES: CN(C(=O)c1cccc2ccccc12)c1sc2c(c1C#N)CCCC2]

Following general procedure D, JNK-5A (100 mg, 0.30 mmol), reacted with iodomethane (22.47 μL, 0.36 mmol) to afford 86 mg (83%) of the title compound as an oil after column chromatography (EtOAc in Hexane: 0-20%).

### Synthesis of N-(3-cyano-4,5,6,7-tetrahydrobenzo[b]thiophen-2-yl)-N-ethyl-1-naphthamide (PKL-B30)

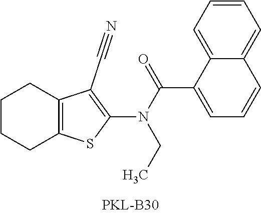 [SMILES: CCN(C(=O)c1cccc2ccccc12)c1sc2c(c1C#N)CCCC2]

Following general procedure D, JNK-5A (100 mg, 0.30 mmol), reacted with iodoethane (26.76 μL, 0.36 mmol) to afford 70 mg (65%) of the title compound as an oil after column chromatography (EtOAc in Hexane: 0-20%).

### Synthesis of N-(3-cyano-4,5,6,7-tetrahydrobenzo[b]thiophen-2-yl)-N-(pyridin-3-ylmethyl)-1-naphthamide (PKL-B32)

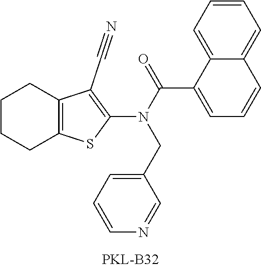 [SMILES: N#Cc1c(N(Cc2cccnc2)C(=O)c2cccc3ccccc23)sc2c1CCCC2]

Following general procedure D, JNK-5A (100 mg, 0.30 mmol), reacted with 3-(chloromethyl) pyridine hydrochloride (42.21 mg, 0.36 mmol) to afford 70 mg (55%) of the title compound as an oil after column chromatography (EtOAc in Hexane: 0-20%).

¹H NMR (500 MHz, CDCl₃) δ 8.52 (d, J=4.9 Hz, 1H), 8.48-8.41 (m, 1H), 7.95 (d, J=8.4 Hz, 1H), 7.87-7.82 (m, 1H), 7.74 (d, J=8.4 Hz, 2H), 7.50 (t, J=7.7 Hz, 1H), 7.43 (t, J=7.4 Hz, 2H), 7.28 (s, 2H), 5.16 (s, 2H), 2.32 (d, J=18.9 Hz, 5H), 1.60 (s, 5H). ¹³C NMR (126 MHz, CDCl₃) δ 170.75, 150-11, 149.70, 137.01, 135.97, 134.29, 134.27, 133.37, 132.45, 131.63, 130.31, 130.00, 128.30, 127.34, 126.60, 125.21, 125.12, 124.51, 123.85, 113.16, 109.10, 31.89, 24.40, 24.08, 22.70, 22.54.

A schematic view of the synthesis of N-(3-cyano-4,5,6,7-tetrahydrobenzo[b]thiophen-2-yl)naphthalene-1-sulfonamide analogues can be seen in FIG. 2 and the synthesis procedure for the specific compounds follows below.

### Synthesis of N-(3-cyano-4,5,6,7-tetrahydrobenzo[b]thiophen-2-yl)naphthalene-1-sulfonamide (PKL-A17)

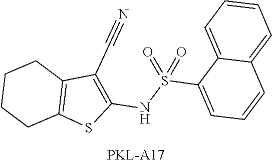 [SMILES: N#Cc1c(NS(=O)(=O)c2cccc3ccccc23)sc2c1CCCC2]

An oven-dried 100 mL round-bottom flask was charged with intermediate JNK-1 (1 eq), 1-sulfonapthoyl chloride (1.2 equiv.) and pyridine as solvent and stirred under an N₂ atmosphere for 12 h. Reaction was monitored by TLC analysis. The solvent was removed under reduced pressure when the reaction was finished. Purification was performed by silica column chromatography (hand column) over silica gel eluting with a gradient of hexane/EtOAc (8:2 to 7:3). The product fractions were pooled and evaporated to afford the title compound with a yield of 80%.

¹H NMR (500 MHz, CDCl₃) δ 7.74 (d, J=8.4 Hz, 1H), 7.37 (d, J=8.2 Hz, 1H), 7.21 (dd, J=5.3, 7.8 Hz, 2H), 6.81 (dt, J=7.2, 22.9 Hz, 2H), 6.73 (t, J=7.8 Hz, 1H), 1.62-1.54 (m, 4H), 0.74 (dq, J=5.7, 6.6, 11.3 Hz, 4H). ¹³C NMR (126 MHz, CDCl₃) δ 140.19, 139.08, 138.95, 138.25, 135.18, 134.33, 133.45, 132.75, 132.32, 129.76, 129.51, 129.48, 118.46, 29.02, 28.86, 27.54, 26.60.

### Synthesis of N-(3-cyano-4,5,6,7-tetrahydrobenzo[b]thiophen-2-yl)-N-(prop-2-yn-1-yl)naphthalene-1-sulfonamide (PKL-A53)

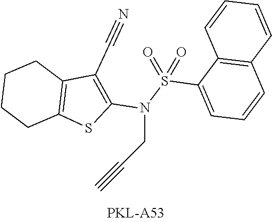 [SMILES: C#CCN(c1sc2c(c1C#N)CCCC2)S(=O)(=O)c1cccc2ccccc12]

Potassium carbonate (514 mg, 3.72 mmol) and propargyl bromide (0.754 mL, 7.44 mmol) was added to a solution of PKL-A17 (207 mg, 0.744 mmol) in DMF (0.07 M) in an oven dried round bottom flask. The mixture was heated to 60° C. and the reaction mixture was poured into ice water. After cooling to room temperature, the mixture was extracted with EtOAc (3 times) and the combined organic layers were washed with 1 N HCl (1 time) and 1 M LiCl (3 times). The mixture was dried over Na₂SO₄ and concentrated. The residue was purified by column chromatography using silica and a gradient of hexane/EtOAc (3:1 to 2:1) to obtain the desired product.

¹H NMR (500 MHz, DMSO) δ 8.43 (d, J=8.6 Hz, 2H), 8.27 (dd, J=1.1, 7.5 Hz, 1H), 8.21-8.17 (m, 1H), 7.78-7.70 (m, 3H), 4.63 (d, J=2.4 Hz, 2H), 3.41 (d, J=2.6 Hz, 1H), 2.69 (t, J=5.4 Hz, 2H), 2.53 (d, J=5.1 Hz, 2H), 1.80 (dd, J=4.1, 7.0 Hz, 4H). ¹³C NMR (126 MHz, DMSO) δ 162.79, 144.93, 138.29, 136.47, 134.42, 134.21, 132.07, 132.01, 129.63, 128.91, 128.53, 127.67, 125.10, 124.71, 113.04, 111.62, 78.47, 77.75, 42.91, 24.67, 24.12, 22.73, 21.85.

### Synthesis of ethyl N-(3-cyano-4,5,6,7-tetrahydrobenzo[b]thiophen-2-yl)-N-(naphthalen-1-ylsulfonyl)glycinate (PKL-A43)

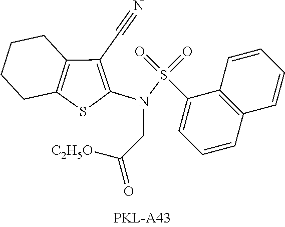

Potassium carbonate (45 mg, 0.32 mmol) ethyl bromoacetate solution (0.0374 mL, 0.32 mmol) and TBAB as a Catalyst (17 mg, 0.05 mmol) were added to a solution of JNK-1 (100 mg, 0.27 mmol) in 7 ml dry THF in an oven dried round bottom flask. The reaction mixture is stirred overnight. The reaction mixture was then poured into ice water. After cooling to room temperature, the mixture was extracted with EtOAc (3 times). The EtOAc layer was dried over Na₂SO₄ and concentrated. The residue was purified by column chromatography using silica to obtain the title compound.

¹H NMR (500 MHz, DMSO) δ 8.40 (t, J=7.9 Hz, 2H), 8.24 (dd, J=1.1, 7.5 Hz, 1H), 8.18 (dd, J=1.7, 7.8 Hz, 1H), 7.76-7.68 (m, 3H), 4.67 (s, 2H), 4.04 (q, J=7.1 Hz, 2H), 2.75-2.64 (m, 2H), 2.50-2.42 (m, 2H), 1.86-1.73 (m, 4H), 1.13 (t, J=7.1 Hz, 3H). ¹³C NMR (126 MHz, DMSO) δ 168.10, 145.87, 138.14, 136.25, 134.54, 134.04, 132.88, 131.56, 129.60, 128.79, 128.60, 127.54, 125.09, 124.65, 112.78, 111.32, 61.68, 55.38, 53.82, 24.58, 24.08, 22.75, 21.86, 14.21.

### Synthesis of N-(3-cyano-4,5,6,7-tetrahydrobenzo[b]thiophen-2-yl)-N-methylnaphthalene-1-sulfon-amide (PKL-A66)

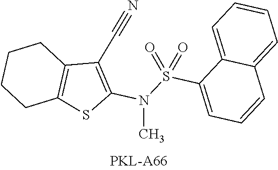 [SMILES: CN(c1sc2c(c1C#N)CCCC2)S(=O)(=O)c1cccc2ccccc12]

Potassium carbonate (514 mg, 3.72 mmol) and methyl iodide (0.754 mL, 7.44 mmol) was added to a solution of JNK-1 (207 mg, 0.744 mmol) in DMF (0.07 M) in an oven dried round bottom flask. The reaction mixture was stirred for 30 min and poured into ice water. After cooling to room temperature, the mixture was extracted with EtOAc (3 times) and the combined organic layers were washed with 1 N HCl (1 time) and 1 M LiCl (3 times). The mixture was dried over Na₂SO₄ and concentrated. The residue was purified by column chromatography using silica to obtain title compound.

¹H NMR (500 MHz, CDCl₃) δ 8.48 (dd, J=3.6, 6.9 Hz, 1H), 8.18 (dd, J=1.6, 7.5 Hz, 1H), 8.07 (d, J=8.3 Hz, 1H), 7.89-7.84 (m, 1H), 7.55-7.46 (m, 3H), 3.28-3.22 (m, 3H), 2.58 (t, J=6.1 Hz, 2H), 2.44-2.37 (m, 2H), 1.75 (tt, J=5.8, 14.3 Hz, 4H). ¹³C NMR (126 MHz, CDCl₃) δ 135.07, 134.49, 133.43, 133.37, 130.68, 130.42, 128.07, 127.83, 127.20, 126.03, 123.99, 123.09, 39.27, 23.73, 23.13, 21.84, 20.87.

### Synthesis of N-benzyl-N-(3-cyano-4,5,6,7-tetrahydrobenzo[b]thiophen-2-yl)naphthalene-1-sulfon-amide (PKL-A44)

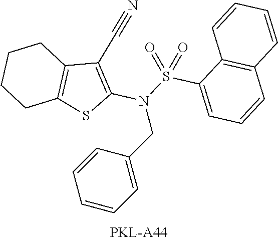 [SMILES: N#Cc1c(N(Cc2ccccc2)S(=O)(=O)c2cccc3ccccc23)sc2c1CCCC2]

Potassium carbonate (514 mg, 3.72 mmol) and benzyl bromide (0.754 mL, 7.44 mmol) were added to a solution of JNK-1 (207 mg, 0.744 mmol) in DMF (0.07 M) in an oven dried round bottom flask. The reaction mixture was stirred for overnight and poured into ice water. After cooling to room temperature, the mixture was extracted with EtOAc (3 times) and the combined organic layers were washed with 1 N HCl (1 time) and 1 M LiCl (3 times). The mixture was dried over Na₂SO₄ and concentrated. The residue was purified by column chromatography using silica to obtain title compound.

¹H NMR (500 MHz, DMSO) δ 8.46 (dd, J=8.4, 15.6 Hz, 2H), 8.34 (dd, J=1.1, 7.4 Hz, 1H), 8.20 (dd, J=1.4, 8.2 Hz, 1H), 7.82-7.68 (m, 3H), 7.39-7.30 (m, 3H), 7.26 (dd, J=1.9, 7.6 Hz, 2H), 4.84 (s, 2H), 2.61-2.57 (m, 2H), 2.42 (t, J=5.5 Hz, 2H), 1.74 (p, J=6.1 Hz, 4H). ¹³C NMR (126 MHz, DMSO-H6) 5 170.82, 145.85, 137.48, 136.35, 135.14, 134.50, 134.25, 132.06, 131.93, 129.65, 128.99, 128.81, 128.71, 128.67, 128.62, 128.50, 127.68, 127.08, 126.88, 125.26, 124.71, 113.19, 111.28, 60.23, 56.48, 24.55, 24.05, 22.67, 21.75, 21.23, 14.55.

### Synthesis of N-(3-cyano-4,5,6,7-tetrahydrobenzo[b]thiophen-2-yl)-N-(2-oxo-2-phenylethyl) naphthalene-1-sulfonamide (PKL-A61)

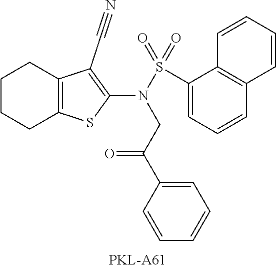 [SMILES: N#Cc1c(N(CC(=O)c2ccccc2)S(=O)(=O)c2cccc3ccccc23)sc2c1CCCC2]

Potassium carbonate (514 mg, 3.72 mmol) and 2-bromoacetophenone (0.754 mL, 7.44 mmol) were added to a solution of JNK-1 (207 mg, 0.744 mmol) in DMF (0.07 M) in an oven dried round bottom flask. The reaction mixture was stirred for 30 min and poured into ice water. After cooling to room temperature, the mixture was extracted with EtOAc (3 times) and the combined organic layers were washed with 1 N HCl (1 time) and 1 M LiCl (3 times). The mixture was dried over Na₂SO₄ and concentrated. The residue was purified by column chromatography using silica to obtain title compound.

¹H NMR (500 MHz, CDCl₃) δ 8.27 (d, J=8.4 Hz, 1H), 7.98 (d, J=7.7 Hz, 2H), 7.71 (dd, J=8.2, 4.0 Hz, 2H), 7.60-7.50 (m, 3H), 7.44 (dt, J=8.1, 4.1 Hz, 3H), 7.29 (dd, J=8.2, 7.1 Hz, 1H), 5.38 (s, 2H), 2.35 (s, 3H), 2.26 (s, 3H), 1.55 (p, J=3.2 Hz, 4H). ¹³C NMR (126 MHz, CDCl₃) δ 191.36, 170.11, 149.85, 135.59, 133.56, 133.08, 132.52, 132.25, 131.62, 129.35, 129.00, 127.89, 127.14, 126.93, 126.26, 125.50, 124.88, 123.85, 123.47, 112.47, 107.71, 55.99, 23.28, 22.98, 21.66, 21.63, 21.52, 20.63.

### Synthesis of N-(3-cyano-4,5,6,7-tetrahydrobenzo[b]thiophen-2-yl)-N-(pyridin-3-ylmethyl) naphthalene-1-sulfonamide (PKL-A58)

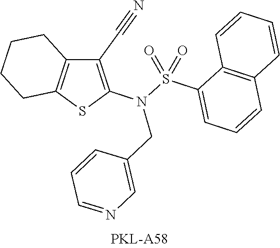 [SMILES: N#Cc1c(N(Cc2cccnc2)S(=O)(=O)c2cccc3ccccc23)sc2c1CCCC2]

Potassium carbonate (514 mg, 3.72 mmol) and 2-chloropyridine (0.754 mL, 7.44 mmol) were added to a solution of JNK-1 (207 mg, 0.744 mmol) in DMF (0.07 M) in an oven dried round bottom flask. The reaction mixture was stirred for 30 min and poured into ice water. After cooling to room temperature, the mixture was extracted with EtOAc (3 times) and the combined organic layers were washed with 1 N HCl (1 time) and 1 M LiCl (3 times). The mixture was dried over Na₂SO₄ and concentrated. The residue was purified by column chromatography using silica to obtain title compound.

¹H NMR (500 MHz, CDCl₃) δ 8.52 (d, J=4.9 Hz, 1H), 8.48-8.41 (m, 1H), 7.95 (d, J=8.4 Hz, 1H), 7.87-7.82 (m, 1H), 7.74 (d, J=8.4 Hz, 2H), 7.50 (t, J=7.7 Hz, 1H), 7.43 (t, J=7.4 Hz, 2H), 7.28 (s, 2H), 5.16 (s, 2H), 2.32 (d, J=18.9 Hz, 5H), 1.60 (s, 5H). ¹³C NMR (126 MHz, CDCl₃) δ 170.75, 150-11, 149.70, 137.01, 135.97, 134.29, 134.27, 133.37, 132.45, 131.63, 130.31, 130.00, 128.30, 127.34, 126.60, 125.21, 125.12, 124.51, 123.85, 113.16, 109.10, 31.89, 24.40, 24.08, 22.70, 22.54.

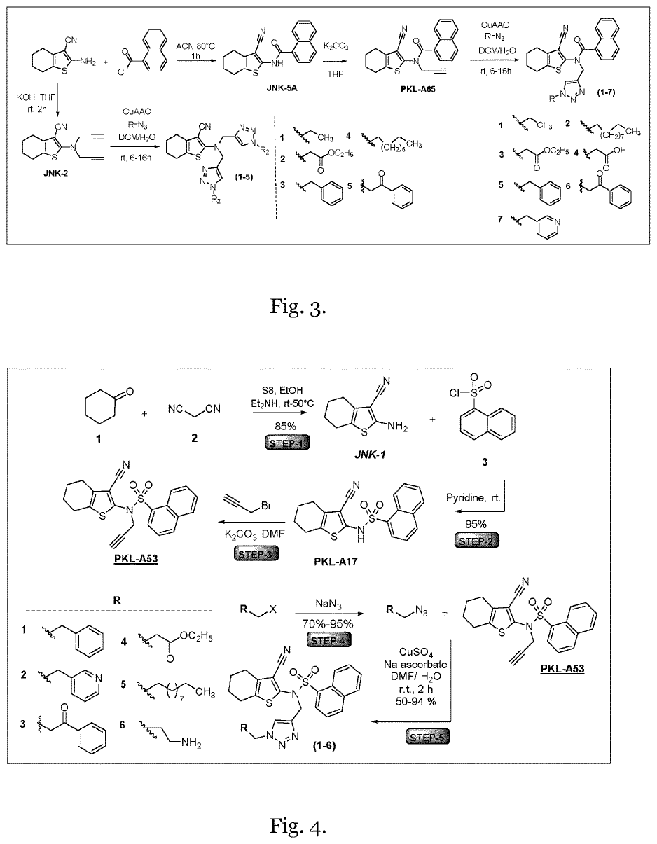

A schematic view of the synthesis of triazole analogues of N-(3-cyano-4,5,6,7-tetrahydrobenzo[b]thiophen-2-yl)-1-naphthamide can be seen in FIG. 3 and the synthesis procedure for the specific compounds follows below.

### Synthesis of N-(3-cyano-4,5,6,7-tetrahydrobenzo[b]thiophen-2-yl)-N-(prop-2-yn-1-yl)-1-naphthamide (PKL-A65)

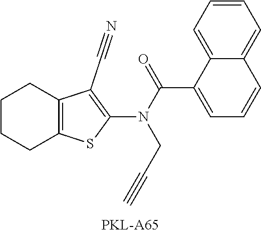 [SMILES: C#CCN(C(=O)c1cccc2ccccc12)c1sc2c(c1C#N)CCCC2]

Following general procedure D, JNK-5A (500 mg, 1.5 mmol), was reacted with propargyl bromide solution (0.013 ml, 1.8 mmol), to afford 480 mg (81%) of the title compound as a yellow liquid after column chromatography (EtOAc in Hexane: 0-10%). ¹H NMR (500 MHz, CDCl3) δ 7.98 (d, J=8.4 Hz, 1H), 7.75 (d, J=7.2 Hz, 3H), 7.48 (dt, J=34.2, 7.3 Hz, 3H), 7.32 (s, 1H), 4.72 (s, 0H), 2.42 (s, 4H), 2.33 (s, 1H), 1.65 (s, 4H). ¹³C NMR (126 MHz, CDCl₃) δ 135.16, 133.23, 132.31, 129.26, 127.20, 126.29, 125.57, 124.99, 124.75, 124.14, 124.05, 123.54, 112.27, 73.22, 66.95, 28.67, 23.50, 23.13, 21.62, 20.66, 18.71.

### Synthesis of N-((1-benzyl-1H-1,2,3-triazol-4-yl)methyl)-N-(3-cyano-4,5,6,7-tetrahydro benzo[b]thiophen-2-yl)-1-naphthamide (PKL-B4)

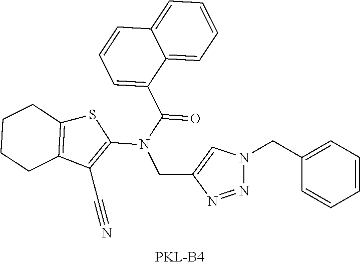 [SMILES: N#Cc1c(N(Cc2cn(Cc3ccccc3)nn2)C(=O)c2cccc3ccccc23)sc2c1CCCC2]

Following general procedure D, PKL-A65 (90 mg, 0.24 mmol), reacted with benzyl azide (38.8 mg, 0.29 mmol) to afford 75 mg (61%) of the title compound as a white solid after column chromatography (EtOAc in Hexane: 0-40%). ¹H NMR (500 MHz, CDCl₃) δ 7.81 (s, 1H), 7.72 (d, J=7.4 Hz, 2H), 7.43-7.38 (m, 2H), 7.33 (d, J=6.9 Hz, 3H), 7.25 (d, J=16.4 Hz, 3H), 7.19 (t, J=2.3 Hz, 1H), 5.51 (s, 2H), 5.16 (s, 2H), 2.45-2.20 (m, 3H), 1.59 (s, 4H). ¹³C NMR (126 MHz, CDCl₃) δ 169.68, 142.24, 135-10, 133.56, 133.09, 132.28, 131.42, 129.11, 128.94, 128.13, 127.76, 127.17, 127.09, 126.08, 125.44, 124.14, 123.47, 112.21, 53.28, 23.43, 23.04, 21.55, 20.59.

### Synthesis of N-(3-cyano-4,5,6,7-tetrahydrobenzo[b]thiophen-2-yl)-N-((1-(2-oxo-2-phenyl ethyl)-1H-1,2,3-triazol-4-yl)methyl)-1-naphthamide (PKL-B31)

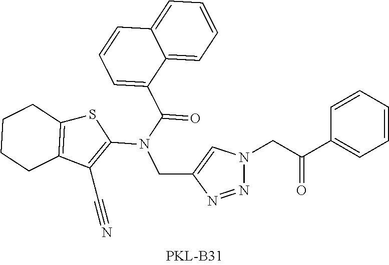 [SMILES: N#Cc1c(N(Cc2cn(CC(=O)c3ccccc3)nn2)C(=O)c2cccc3ccccc23)sc2c1CCCC2]

Following general procedure D, PKL-A65 (100 mg, 0.30 mmol), reacted with 2-azido-1-phenylethan-1-one (52.2 mg, 0.32 mmol) to afford 95 mg (66%) of the title compound as a white solid after column chromatography (EtOAc in Hexane: 0-40%).

¹H NMR (500 MHz, CDCl₃) δ 7.95 (d, J=7.8 Hz, 3H), 7.72 (d, J=8.2 Hz, 2H), 7.60 (d, J=7.4 Hz, 1H), 7.53-7.39 (m, 5H), 7.29 (s, 1H), 5.84 (s, 2H), 5.22 (s, 2H), 2.33 (s, 4H), 1.59 (s, 4H). ¹³C NMR (126 MHz, CDCl₃) δ 189.98, 170.85, 143-19, 136.27, 134.60, 134.11, 133.99, 133.31, 132.59, 130.09, 129.22, 128.43, 128.15, 127.26, 126.52, 126.10, 125.37, 125.06, 124.53, 113.35, 109.57, 55.63, 31.60, 24.48, 24.10, 22.67, 22.59.

### Synthesis of ethyl2-(4-((N-(3-cyano-4,5,6,7-tetrahydrobenzo[b]thiophen-2-yl)-1-naphth amido)methyl)-1H-1,2,3-triazol-1-yl)acetate (PKL-B28)

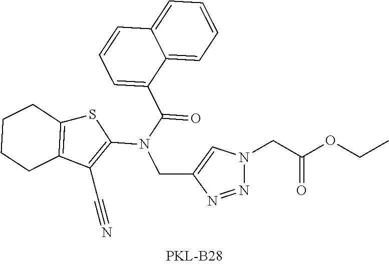 [SMILES: CCOC(=O)Cn1cc(CN(C(=O)c2cccc3ccccc23)c2sc3c(c2C#N)CCCC3)nn1]

Following general procedure D, PKL-A65 (200 mg, 0.54 mmol), reacted with ethyl 2-azidoacetate (76.6 mg, 0.59 mmol) to afford 235 mg (88%) of the title compound as a white solid after column chromatography (EtOAc in Hexane: 0-50%).

¹H NMR (500 MHz, CDCl₃) δ 7.99 (s, 1H), 7.91 (d, J=8.4 Hz, 1H), 7.72 (d, J=8.2 Hz, 2H), 7.50-7.40 (m, 3H), 7.28 (s, 1H), 5.21 (s, 2H), 5.14 (s, 2H), 4.23 (q, J=7.2 Hz, 2H), 2.32 (s, 4H), 1.58 (d, J=9.9 Hz, 5H), 1.25 (t, J=7.2 Hz, 3H). ¹³C NMR (126 MHz, CDCl₃) δ 171.17, 170.81, 166.13, 149.60, 143.30, 136.21, 134-11, 133.32, 132.50, 130.15, 130.02, 128.18, 127.19, 126.50, 125.57, 125.28, 125.13, 124.51, 113.29, 62.47, 60.41, 51.02, 24.46, 24.09, 22.58, 21.62, 14.13.

### Synthesis of 2-(4-((N-(3-cyano-4,5,6,7-tetrahydrobenzo[b]thiophen-2-yl)-1-naphthamido)methyl)-1H-1,2,3-triazol-1-yl)acetic acid (PKL-B33)

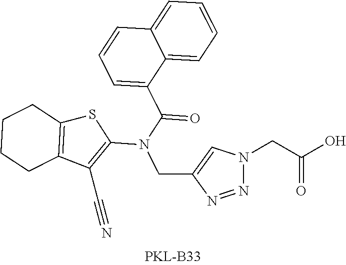 [SMILES: N#Cc1c(N(Cc2cn(CC(=O)O)nn2)C(=O)c2cccc3ccccc23)sc2c1CCCC2]

Following general procedure C, PKL-B28 (80 mg, 0.16 mmol), reacted with Lithium hydroxide (13.44 mg, 0.32 mmol) to afford 49 mg (65%) of the title compound as a yellow solid after column chromatography (EtOAc in Hexane: 0-20%).

¹H NMR (500 MHz, CDCl₃) δ 8.13 (s, 1H), 7.88 (d, J=8.4 Hz, 1H), 7.73 (t, J=7.4 Hz, 2H), 7.49-7.39 (m, 4H), 7.28 (t, J=7.7 Hz, 1H), 5.23 (s, 2H), 5.12 (s, 3H), 2.31 (s, 6H), 1.58 (s, 6H). ¹³C NMR (126 MHz, CDCl₃) δ 171.51, 167.97, 136.49, 134.23, 133.28, 131.99, 130.40, 129.89, 128.24, 127.32, 126.62, 125.24, 125.16, 124.50, 50.79, 24.45, 24.06, 22.55, 21.58.

### Synthesis of 2-(4-((N-(3-cyano-4,5,6,7-tetrahydrobenzo[b]thiophen-2-yl)-1-naphthamido)methyl)-1H-1,2,3-triazol-1-yl)acetic acid (PKL-B34)

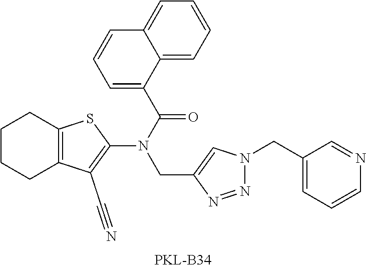 [SMILES: N#Cc1c(N(Cc2cn(Cc3cccnc3)nn2)C(=O)c2cccc3ccccc23)sc2c1CCCC2]

Following general procedure D, PKL-A65 (100 mg, 0.30 mmol), reacted with 3-(azidomethyl) pyridine (43.4 mg, 0.32 mmol) to afford 97 mg (71%) of the title compound as a white solid after column chromatography (EtOAc in Hexane: 0-40%).

¹H NMR (500 MHz, CDCl₃) δ 8.60 (s, 1H), 7.87-7.79 (m, 1H), 7.76-7.68 (m, 2H), 7.55 (t, J=9.4 Hz, 1H), 7.46-7.38 (m, 3H), 7.31-7.23 (m, 2H), 7.18 (d, J=8.8 Hz, 1H), 5.53 (s, 2H), 5.16 (s, 2H), 2.31 (d, J=21.9 Hz, 4H), 1.59 (s, 5H). ¹³C NMR (126 MHz, CDCl₃) δ 170.81, 170.77, 150.20, 149.45, 149-14, 143.62, 136.24, 135.79, 134.16, 133.32, 132.34, 130.75, 130.23, 128.23, 127.15, 126.53, 125.21, 125.13, 124.49, 51.72, 46.67, 24.47, 24.06, 22.56, 21.59.

### N-(3-cyano-4,5,6,7-tetrahydrobenzo[b]thiophen-2-yl)-N-((1-decyl-1H-1,2,3-triazol-4-yl)methyl)-1-naphthamide (PKL-B35)

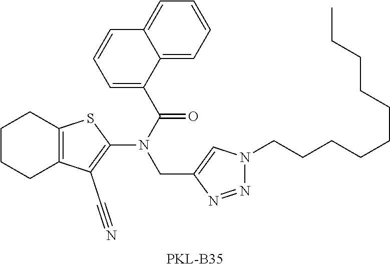 [SMILES: CCCCCCCCCCn1cc(CN(C(=O)c2cccc3ccccc23)c2sc3c(c2C#N)CCCC3)nn1]

Following general procedure D, PKL-A65 (100 mg, 0.39 mmol), reacted with azidodecane (79.27 mg, 0.43 mmol) to afford 140 mg (57%) of the title compound as a white solid after column chromatography (EtOAc in Hexane: 0-40%).

¹H NMR (500 MHz, CDCl₃) δ 7.91 (d, J=8.2 Hz, 1H), 7.83 (s, 1H), 7.73 (d, J=8.1 Hz, 2H), 7.44 (dq, J=15.1, 7.4 Hz, 3H), 7.29 (s, 1H), 5.18 (s, 2H), 4.32 (t, J=7.0 Hz, 2H), 2.32 (s, 4H), 1.87 (s, 2H), 1.59 (s, 2H), 1.27 (s, 4H), 1.24-1.13 (m, 11H), 0.81 (t, J=6.8 Hz, 3H). ¹³C NMR (126 MHz, CDCl₃) δ 170.75, 142.74, 136.11, 134.07, 133.33, 132.51, 130.15, 129.97, 128.22, 127.10, 126.47, 125.21, 124.51, 77.28, 77.03, 76.77, 50.53, 31.87, 30.30, 29.51, 29.44, 29.28, 29.03, 26.51, 24.47, 24.07, 22.68, 22.58, 21.62, 14.12.

### Synthesis of 2-(di(prop-2-yn-1-yl)amino)-4,5,6,7-tetrahydrobenzo[b]thiophene-2-carbonitrile (JNK-2)

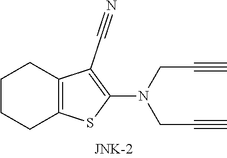 [SMILES: C#CCN(CC#C)c1sc2c(c1C#N)CCCC2]

A solution of JNK-1 (1 g, 5.61 mmol), potassium hydroxide (346.23 mg, 6.17 mmol) and TBAB (Catalytic amount 0.2 equiv.) in THF was stirred for 30 min at room temperature. Then propargyl bromide solution (0.051 ml, 6.73 mmol) was added, and the reaction mixture was stirred at room temperature for 48 h. The residue was partitioned between water and ethyl acetate and the aqueous extracted with further ethyl acetate. The combined organic extracts were washed with brine, dried over magnesium sulphate and evaporated to dryness under reduced pressure to give the desired product as a yellow liquid.

### Synthesis of 2-(bis((1-(2-oxo-2-phenylethyl)-1H-1,2,3-triazol-4-yl)methyl) amino)-4,5,6,7-tetrahydro-benzo[b]thiophene-3-carbonitrile (PKL-B86)

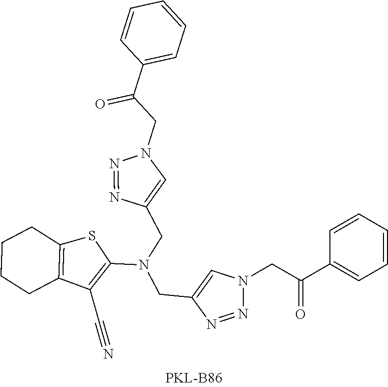 [SMILES: N#Cc1c(N(Cc2cn(CC(=O)c3ccccc3)nn2)Cc2cn(CC(=O)c3ccccc3)nn2)sc2c1CCCC2]

Following general procedure D, JNK-2 (100 mg, 0.39 mmol), reacted with 2-azido-1-phenylethan-1-one (30.74 mg, 0.43 mmol) to afford no mg (49%) of the title compound as a white solid after column chromatography (EtOAc in Hexane: 0-60%).

¹H NMR (500 MHz, CDCl₃) δ 7.81 (d, J=1.3 Hz, 2H), 7.79 (d, J=1.4 Hz, 2H), 7.65 (s, 2H), 7.59-7.54 (m, 2H), 7.42-7.37 (m, 4H), 5.80 (s, 4H), 4.83 (s, 4H), 2.43 (dd, J=6.5, 4.2 Hz, 4H), 1.75-1.67 (m, 4H). ¹³C NMR (126 MHz, CDCl₃) δ 190.38, 162.52, 134.51, 133.88, 133.77, 129.13, 128.02, 125.38, 121.28, 87.03, 55.62, 49.30, 24.62, 24.14, 23.25, 22.17.

### Synthesis of 2-(bis((1-benzyl-1H-1,2,3-triazol-4-yl)methyl)amino)-4,5,6,7-tetrahydrobenzo[b]thiophene-3-carbonitrile (PKL-B60)

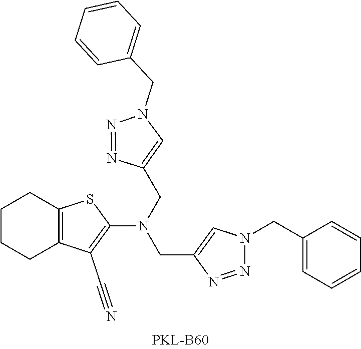 [SMILES: N#Cc1c(N(Cc2cn(Cc3ccccc3)nn2)Cc2cn(Cc3ccccc3)nn2)sc2c1CCCC2]

Following general procedure D, JNK-2 (200 mg, 0.78 mmol), reacted with (azidomethyl)benzene (125.64 mg, 0.94 mmol)) to afford 220 mg (54%) of the title compound as a white solid after column chromatography (EtOAc in Hexane: 0-60%).

¹H NMR (500 MHz, CDCl₃) δ 7.81 (d, J=1.3 Hz, 2H), 7.79 (d, J=1.4 Hz, 2H), 7.65 (s, 2H), 7.59-7.54 (m, 2H), 7.42-7.37 (m, 4H), 5.80 (s, 4H), 4.83 (s, 4H), 2.43 (dd, J=6.5, 4.2 Hz, 4H), 1.75-1.67 (m, 4H). ¹³C NMR (126 MHz, CDCl₃) δ 190.38, 162.52, 134.51, 133.88, 133.77, 129.13, 128.02, 125.38, 121.28, 87.03, 55.62, 49.30, 24.62, 24.14, 23.25, 22.17.

### Synthesis of diethyl 2,2′-((((3-cyano-4,5,6,7-tetrahydrobenzo[b]thiophen-2-yl))azanediyl)bis(methylene))bis(1H-1,2,3-triazole-4,1-diyl))diacetate (PKL-B40)

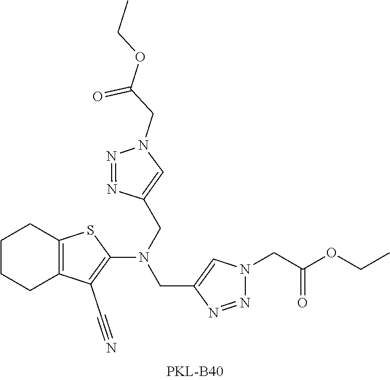 [SMILES: CCOC(=O)Cn1cc(CN(Cc2cn(CC(=O)OCC)nn2)c2sc3c(c2C#N)CCCC3)nn1]

Following general procedure D, JNK-2 (200 mg, 0.78 mmol), reacted with ethyl 2-azidoacetate (111.68 mg, 0.86 mmol) to afford 230 mg (57%) of the title compound as a white solid after column chromatography (EtOAc in Hexane: 0-60%).

¹H NMR (500 MHz, CDCl₃) δ 7.67 (s, 2H), 5.08 (s, 4H), 4.75 (s, 4H), 4.18 (q, J=7.1 Hz, 4H), 2.43 (dt, J=11.4, 5.6 Hz, 4H), 1.70 (dq, J=11.9, 6.5, 5.9 Hz, 4H), 1.22 (t, J=7.1 Hz, 7H). ¹³C NMR (126 MHz, CDCl₃) δ 166.32, 162.47, 143.59, 133.78, 124.84, 121.55, 117.53, 87.70, 62.44, 50.90, 48.79, 24.60, 24.14, 23.22, 22.15, 14.04.

### Synthesis of 2-(bis((1-decyl-1H-1,2,3-triazol-4-yl)methyl)amino)-4,5,6,7-tetrahydro benzo[b]thiophene-3-carbonitrile (PKL-B8.5)

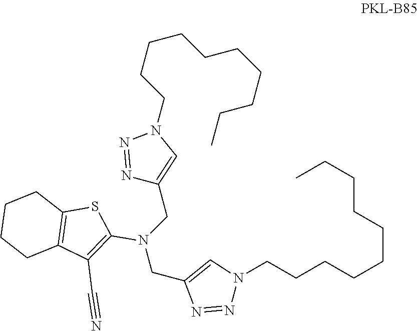 [SMILES: CCCCCCCCCCn1cc(CN(Cc2cn(CCCCCCCCCC)nn2)c2sc3c(c2C#N)CCCC3)nn1]

Following general procedure D, JNK-2 (100 mg, 0.39 mmol), reacted with azidodecane (79.27 mg, 0.43 mmol) to afford 140 mg (57%) of the title compound as a white solid after column chromatography (EtOAc in Hexane: 0-40%).

¹H NMR (500 MHz, CDCl₃) δ 7.65 (s, 2H), 4.67 (s, 4H), 4.26 (t, J=7.3 Hz, 4H), 2.43 (dt, J=11.9, 5.6 Hz, 4H), 1.82 (t, J=7.3 Hz, 4H), 1.76-1.66 (m, 4H), 1.30-1.22 (m, 11H), 1.18 (s, 20H), 0.80 (t, J=6.8 Hz, 7H). ¹³C NMR (126 MHz, CDCl₃) δ 161.65, 141.75, 132.76, 122.23, 120.35, 116.55, 86.76, 49.44, 47.25, 30.83, 29.27, 28.67, 28.45, 28.37, 28.23, 28.23, 27.96, 25.46, 23.57, 23.10, 22.21, 21.64, 21.13, 13.08.

A schematic view of the synthesis of triazole analogues of N-(3-cyano-4,5,6,7-tetrahydrobenzo[b]thiophen-2-yl)naphthalene-1-sulfonamide can be seen in FIG. 4 and the synthesis procedure for the specific compounds follows below.

### General Procedure for the Synthesis of N-(3-cyano-4,5,6,7-tetrahydrobenzo[b]thiophen-2-yl)-N-(prop-2-yn-1-yl)naphthalene-1-sulfonamide (PKL-A53)

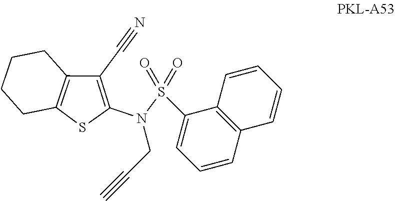 [SMILES: C#CCN(c1sc2c(c1C#N)CCCC2)S(=O)(=O)c1cccc2ccccc12]

Add 514 mg (3.72 mmol) K₂CO₃, 0.754 mL (1.50 g, 7.44 mmol) propargyl bromide to a solution of 207 mg (0.744 mmol) PKL-A17 in DMF (0.07 M) in an oven dried round bottom flask. Heat the mixture to 60° C. Pour the reaction mixture into ice water. After cooling to room temperature, extract the mixture with EtOAc (3×). Wash the combined organic layers with 1 N HCl (1×) and 1 M LiCl (3×). Dry the mixture over Na₂SO₄ and concentrate it. Purify the residue by column chromatography using silica and hexanes/EtOAc 3:1→2:1 to obtain desired product or extract with ether/water, combine ether layer and concentrate the mixture. Purify the residue by column chromatography using silica and hexane/EtOAc 3:1→2:1 to obtain the desired product.

¹H NMR (500 MHz, DMSO) δ 8.43 (d, J=8.6 Hz, 2H), 8.27 (dd, J=1.1, 7.5 Hz, 1H), 8.21-8.17 (m, 1H), 7.78-7.70 (m, 3H), 4.63 (d, J=2.4 Hz, 2H), 3.41 (d, J=2.6 Hz, 1H), 2.69 (t, J=5.4 Hz, 2H), 2.53 (d, J=5.1 Hz, 2H), 1.80 (dd, J=4.1, 7.0 Hz, 4H). ¹³C NMR (126 MHz, DMSO) δ 162.79, 144.93, 138.29, 136.47, 134.42, 134.21, 132.07, 132.01, 129.63, 128.91, 128.53, 127.67, 125.10, 124.71, 113.04, 111.62, 78.47, 77.75, 42.91, 24.67, 24.12, 22.73, 21.85.

### General Procedure for the Synthesis of Azides of Heterocycles, Alkyl and Benzyl Compounds

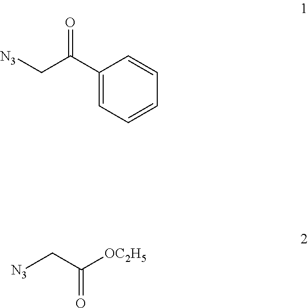 [SMILES: [N-]=[N+]=NCC(=O)c1ccccc1 ; CCOC(=O)CN=[N+]=[N-]]

Method A: Around bottom flask was charged with alpha-halogenated heterocycles (6.5 mmol) in acetonitrile (20 mL) at room temperature. Sodium azide (19.4 mmol) was added to the stirring solution for 4 h. Upon completion, cold water was added and the reaction mixture was extracted with EtOAc (2×20 mL). The organic layers were then dried using anhydrous MgSO4, filtered, and concentrated to obtain corresponding azides with good yield.

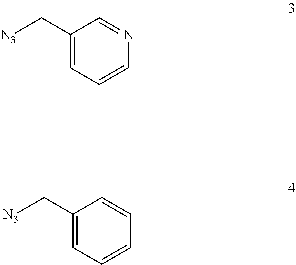 [SMILES: [N-]=[N+]=NCc1cccnc1 ; [N-]=[N+]=NCc1ccccc1]

Method B: To a stirred solution of 10.0 mmol benzyl halides in 100 mL of acetone/H₂O 4:1 (v/v) was added 15.0 mmol (0.98 g) of sodium azide. The reaction mixture was stirred at room temperature for 24 h. After, the reaction was extracted with Et₂O (3×50 mL), dried over Na₂SO₄, and concentrated under reduced pressure to give benzyl azides as pale yellow oils.

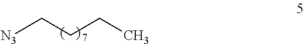

Method C: To a stirred solution of 10.0 mmol alkyl halides in 20 mL of DMF, 12.0 mmol (0.78 g) of sodium azide was added. The reaction mixture was stirred at 70° C. for 24 h in an oil bath. After, the reaction was extracted with Et₂O (3×50 mL), dried over Na₂SO₄, and concentrated under reduced pressure to give alkyl azides as pale yellow oil.

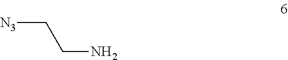

(Method D): To a stirred solution of 10.0 mmol alkyl amine halides in 5 mL of H₂O, 12.0 mmol (0.78 g) of sodium azide was added. The reaction mixture was stirred at 70° C. for 12 h in an oil bath. After, the reaction was extracted with Et₂O (3×50 mL), dried over Na₂SO₄, and concentrated under reduced pressure to give alkyl azides as pale yellow oil.

### General Procedure for the Synthesis of triazole analogues of N-(3-cyano-4,5,6,7-tetrahydrobenzo[b]thiophen-2-yl)naphthalene-1-sulfonamide

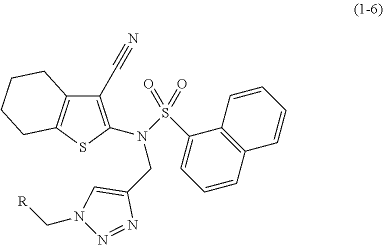

To a solution of PKL-A53 (0.2 g, 0.97 mmol) in mixture of DMF:water (3:2, v/v, 5 mL), a stoichiometric amount of corresponding acetophenone and heterocyclic azides (1.0 mmol) was added. After addition of copper sulphate (0.48 mmol) and sodium L-ascorbate (0.73 mmol), reaction mixture was left for stirring at room temperature for 12 h. On complete utilization of starting material, reaction mixture was adsorbed directly on silica, and subjected to purification by column chromatography to obtain desired products in 65-94% yields (Eluent: hexane:ethyl acetate: 8.0:2.0-1.0:9.0).

### Synthesis of N-((1-benzyl-1H-1,2,3-triazol-4-yl)methyl)-N-(3-cyano-4,5,6,7-tetrahydrobenzo[b]thiophen-2-yl)naphthalene-1-sulfonamide (PKL-A55)

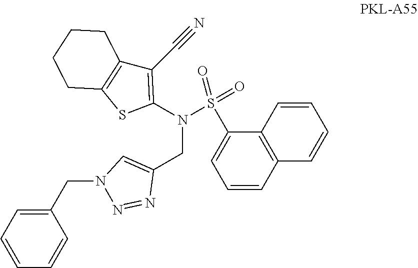 [SMILES: N#Cc1c(N(Cc2cn(Cc3ccccc3)nn2)S(=O)(=O)c2cccc3ccccc23)sc2c1CCCC2]

Following general procedure D, N-(3-cyano-4,5,6,7-tetrahydrobenzo[b]thiophen-2-yl)-N-(prop-2-yn-1-yl)naphthalene-1-sulfonamide (PKL-A53) reacted with benzyl azide to afford (70%) of the title compound as a white solid after column chromatography (EtOAc in Hexane: 0-70%).

¹H NMR (500 MHz, CDCl₃) δ 8.60 (dd, J=3.5, 6.6 Hz, 1H), 8.24 (dd, J=1.7, 7.5 Hz, 1H), 8.13 (d, J=8.2 Hz, 1H), 7.95 (dd, J=3.5, 6.6 Hz, 1H), 7.60 (ddd, J=1.9, 4.4, 8.4 Hz, 3H), 7.53 (t, J=7.9 Hz, 1H), 7.39-7.34 (m, 3H), 7.23 (t, J=4.4 Hz, 2H), 5.48 (s, 2H), 4.95 (s, 2H), 2.61-2.53 (m, 2H), 2.41 (t, J=6.1 Hz, 2H), 1.80- 1.70 (m, 4H). ¹³C NMR (126 MHz, CDCl₃) δ 143.96, 141.81, 136.72, 134.62, 133.39, 131.42, 130.44, 128.08, 127.95, 127.74, 127.46, 127.09, 126.17, 123.93, 123.09, 122.77, 111.66, 110.77, 53.27, 47.76, 28.68, 23.77, 23.07, 21.70, 20.75.

### Synthesis of N-(3-cyano-4,5,6,7-tetrahydrobenzo[b]thiophen-2-yl)-N-((1-(pyridin-3-ylmethyl)-1H-1,2,3-triazol-4-yl)methyl)naphthalene-1-sulfonamide (PKL-A64)

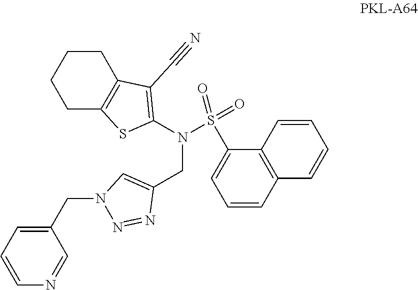 [SMILES: N#Cc1c(N(Cc2cn(Cc3cccnc3)nn2)S(=O)(=O)c2cccc3ccccc23)sc2c1CCCC2]

Following general procedure D, N-(3-cyano-4,5,6,7-tetrahydrobenzo[b]thiophen-2-yl)-N-(prop-2-yn-1-yl)naphthalene-1-sulfonamide (PKL-A53) reacted with 3-(azidomethyl)pyridine to afford (65%) of the title compound as a brown solid after column chromatography (EtOAc in Hexane: 50-100%).

¹H NMR (500 MHz, CDCl₃) δ 8.67-8.54 (m, 3H), 8.24 (d, J=7.3 Hz, 1H), 8.13 (d, J=8.2 Hz, 1H), 7.95 (dt, J=2.2, 5.7 Hz, 1H), 7.65-7.59 (m, 3H), 7.58-7.51 (m, 2H), 7.32 (d, J=6.6 Hz, 1H), 5.51 (s, 2H), 4.95 (d, J=1.7 Hz, 2H), 2.57 (t, J=6.1 Hz, 2H), 2.42 (t, J=6.0 Hz, 2H), 1.76 (dq, J=5.4, 19.1 Hz, 4H). ¹³C NMR (126 MHz, CDCl₃) δ 170.14, 149.20, 148.17, 143.92, 142.25, 136.82, 134.77, 134.67, 133.48, 133.38, 131.36, 130.50, 127.99, 127.72, 127.50, 126.21, 123.91, 123.12, 122.92, 122.88, 111.72, 110.88, 59.38, 50.65, 47.79, 23.78, 23.08, 21.69, 20.74, 13.18.

### Synthesis of N-(3-cyano-4,5,6,7-tetrahydrobenzo[b]thiophen-2-yl)-N-((1-(2-oxo-2-phenylethyl)-1H-1,2,3-triazol-4-yl)methyl)naphthalene-1-sulfonamide (PKL-A54)

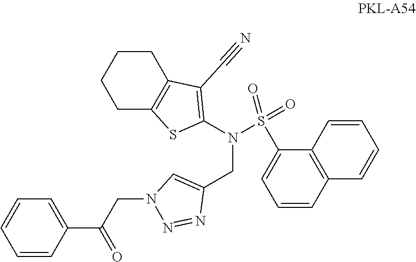 [SMILES: N#Cc1c(N(Cc2cn(CC(=O)c3ccccc3)nn2)S(=O)(=O)c2cccc3ccccc23)sc2c1CCCC2]

Following general procedure D, N-(3-cyano-4,5,6,7-tetrahydrobenzo[b]thiophen-2-yl)-N-(prop-2-yn-1-yl)naphthalene-1-sulfonamide (PKL-A53) reacted with 2-azido-1-phenylethan-1-one to afford (80%) of the title compound as a white solid after column chromatography (EtOAc in Hexane: 0-70%).

¹H NMR (500 MHz, CDCl₃) δ 8.60 (dd, J=3.5, 6.6 Hz, 1H), 8.24 (dd, J=1.7, 7.5 Hz, 1H), 8.13 (d, J=8.2 Hz, 1H), 7.95 (dd, J=3.5, 6.6 Hz, 1H), 7.60 (ddd, J=1.9, 4.4, 8.4 Hz, 3H), 7.53 (t, J=7.9 Hz, 1H), 7.39-7.34 (m, 3H), 7.23 (t, J=4.4 Hz, 2H), 5.48 (s, 2H), 4.95 (s, 2H), 2.61-2.53 (m, 2H), 2.41 (t, J=6.1 Hz, 2H), 1.80-1.70 (m, 4H). ¹³C NMR (126 MHz, CDCl₃) δ 189.98, 143.96, 141.81, 136.72, 134.62, 133.39, 131.42, 130.44, 128.08, 127.95, 127.74, 127.46, 127.09, 126.17, 123.93, 123.09, 122.77, 111.66, 110.77, 53.27, 47.76, 28.68, 23.77, 23.07, 21.70, 20.75.

### Synthesis of ethyl 2-(4-((N-(3-cyano-4,5,6,7-tetrahydrobenzo[b]thiophen-2-yl)naphthalene-1-sulfonamido)methyl)-1H-1,2,3-triazol-1-yl)acetate (PKL-A73)

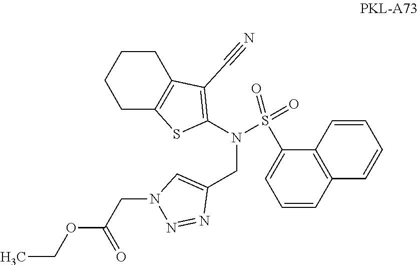 [SMILES: CCOC(=O)Cn1cc(CN(c2sc3c(c2C#N)CCCC3)S(=O)(=O)c2cccc3ccccc23)nn1]

Following general procedure D, N-(3-cyano-4,5,6,7-tetrahydrobenzo[b]thiophen-2-yl)-N-(prop-2-yn-1-yl)naphthalene-1-sulfonamide (PKL-A53) reacted with ethyl 2-azidoacetate to afford (89%) of the title compound as a white solid after column chromatography (EtOAc in Hexane: 0-70%).

¹H NMR (500 MHz, CDCl₃) δ 8.63 (d, J=8.3 Hz, 1H), 8.27 (d, J=7.3 Hz, 1H), 8.15 (d, J=8.2 Hz, 1H), 7.96 (d, J=8.0 Hz, 1H), 7.86 (s, 1H), 7.64 (t, J=8.3 Hz, 2H), 7.55 (t, J=7.8 Hz, 1H), 5.12 (s, 2H), 5.00 (s, 2H), 4.27 (q, J=7.1 Hz, 2H), 2.58 (t, J=5.9 Hz, 2H), 2.43 (t, J=6.0 Hz, 2H), 1.81-1.70 (m, 4H), 1.30 (t, J=7.2 Hz, 3H). ¹³C NMR (126 MHz, CDCl₃) δ 164.97, 144.03, 141.91, 136.75, 134.66, 133.46, 133.42, 131.43, 130.49, 127.98, 127.80, 127.50, 126.18, 124.41, 123.94, 123.13, 111.71, 110.71, 61.44, 49.97, 47.72, 23.78, 23.09, 21.70, 20.76, 13.07, 0.00.

### Synthesis of N-(3-cyano-4,5,6,7-tetrahydrobenzo[b]thiophen-2-yl)-N-((1-decyl-1H-1,2,3-triazol-4-yl)methyl)naphthalene-1-sulfonamide (PKL-A48-1)

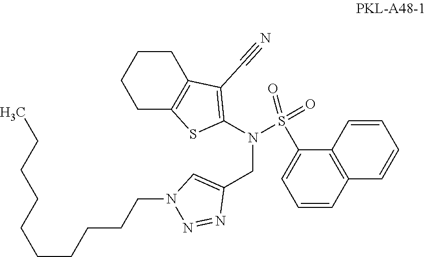 [SMILES: CCCCCCCCCCn1cc(CN(c2sc3c(c2C#N)CCCC3)S(=O)(=O)c2cccc3ccccc23)nn1]

Following general procedure D, N-(3-cyano-4,5,6,7-tetrahydrobenzo[b]thiophen-2-yl)-N-(prop-2-yn-1-yl)naphthalene-1-sulfonamide (PKL-A53) reacted with 1-azidodecane to afford (88%) of the title compound as a white solid after column chromatography (EtOAc in Hexane: 0-65%).

¹H NMR (500 MHz, CDCl₃) δ 8.64 (d, J=8.2 Hz, 1H), 8.26 (d, J=7.3 Hz, 1H), 8.15 (d, J=8.1 Hz, 1H), 7.97 (d, J=7.8 Hz, 1H), 7.67 (s, 1H), 7.66-7.60 (m, 2H), 7.55 (t, J=7.8 Hz, 1H), 4.97 (s, 2H), 4.30 (t, J=7.2 Hz, 2H), 2.57 (t, J=6.0 Hz, 2H), 2.43 (t, J=6.0 Hz, 2H), 1.86 (t, J=7.2 Hz, 2H), 1.82-1.69 (m, 4H), 1.26 (s, 15H), 0.88 (t, J=6.8 Hz, 3H). ¹³C NMR (126 MHz, CDCl₃) δ 144.06, 141.39, 136.71, 134.62, 133.40, 131.49, 130.46, 127.98, 127.80, 127.49, 126.19, 123.98, 123.12, 122.56, 111.74, 110.80, 49.50, 47.96, 30.85, 29.14, 28.48, 28.40, 28.26, 27.96, 25.41, 23.78, 23.09, 21.70, 21.66, 20.76, 13.09.

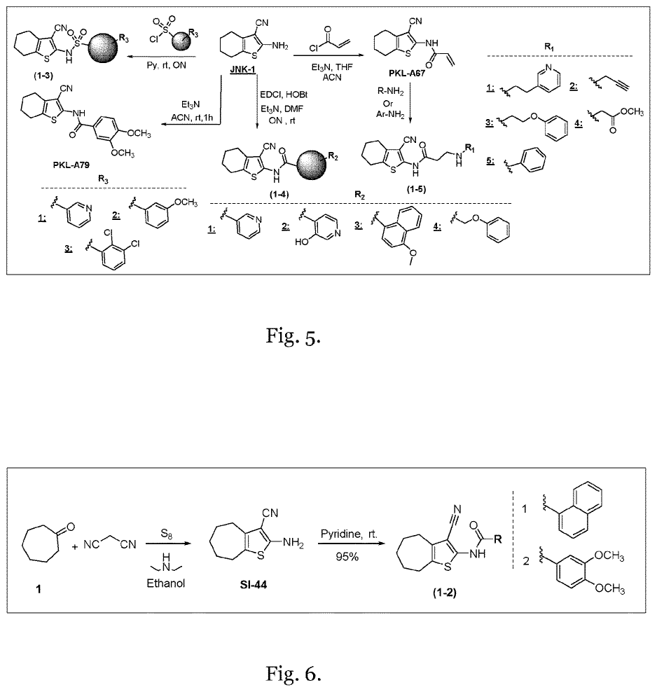

A schematic view of the synthesis of analogues of 2-amino-4,5,6,7-tetrahydrobenzo[b]thiophene-3-carbonitrile can be seen in FIG. 5 and the synthesis procedure for the specific compounds follows below.

### General Procedure for the Synthesis of N-(3-cyano-4,5,6,7-tetrahydrobenzo[b]thiophen-2-yl) acrylamide (PKL-A67)

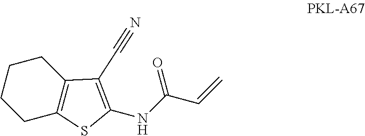 [SMILES: C=CC(=O)Nc1sc2c(c1C#N)CCCC2]

To the solution of JNK-1 (1 equiv.) in THF or ACN (3-5 mL) Et₃N (1.1 equiv.) was added at 0° C. and left at room temperature for 1.5 h. After that, add acryoyl chloride (1.1 equiv.) dropwise to the reaction mixture at 0° C. and stir at room temperature overnight. The reaction mixture was quenched with cold NH₄Cl solution and extracted with EtOAc. The combined organic layers were washed with brine, dried (Na₂SO₄), and concentrated under reduced pressure. Purification by column chromatography (3:1 hexanes/EtOAc) afforded desired compound in 75% yield.

¹H NMR (500 MHz, CDCl₃) δ 8.57 (s, 1H), 6.55 (d, J=16.8 Hz, 1H), 6.39-6.30 (m, 1H), 5.92 (d, J=10.2 Hz, 1H), 2.66 (d, J=5.4 Hz, 2H), 2.60 (d, J=5.4 Hz, 2H), 1.86-1.82 (m, 4H). ¹³C NMR (126 MHz, CDCl₃) δ 160.75, 145.51, 130.05, 129.58, 127.98, 127.48, 113.48, 22.95, 22.05, 21.08.

### General Procedure for the Synthesis of Michael Adducts of N-(3-cyano-4,5,6,7-tetrahydro benzo[b]thiophen-2-yl)acrylamide (1-5)

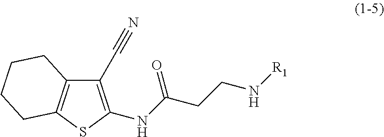

To the solution of A (1 equiv.) in ACN (3-5 mL) Et₃N (1.1 equiv.) was added at 0° C. and left at room temperature for 1.5 h. After that, add the corresponding amine (1.1 equiv.) dropwise to the reaction mixture at 0° C. and stir at room temperature overnight. The reaction mixture was quenched with cold NH4Cl solution and extracted with EtOAc. The combined organic layers were washed with brine, dried (Na₂SO₄), and concentrated under reduced pressure. Purification by column chromatography (3:1 hexanes/EtOAc) afforded desired compound in good yield.

### Synthesis of N-(3-cyano-4,5,6,7-tetrahydrobenzo[b]thiophen-2-yl)-3-(phenylamino)propanamide (PKL-A20)

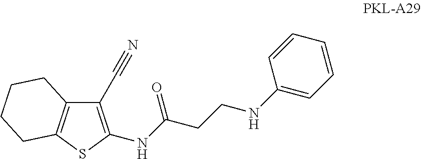 [SMILES: N#Cc1c(NC(=O)CCNc2ccccc2)sc2c1CCCC2]

Following general procedure for the Synthesis of Michael adducts, N-(3-cyano-4,5,6,7-tetrahydrobenzo[b]thiophen-2-yl) acrylamide (PKL-A67) reacted with aniline to afford (80%) of the title compound as a white solid after column chromatography (EtOAc in Hexane: 0-85%).

¹H NMR (500 MHz, CDCl₃) δ 9.56 (s, 1H), 7.23 (t, J=7.7 Hz, 2H), 6.82 (t, J=7.3 Hz, 1H), 6.77 (d, J=8.0 Hz, 2H), 3.58 (t, J=5.9 Hz, 2H), 2.77 (t, J=5.9 Hz, 2H), 2.65-2.60 (m, 2H), 2.56 (t, J=5.8 Hz, 2H), 1.82 (dp, J=3.6, 4.6, 11.2 Hz, 5H). ¹³C NMR (126 MHz, CDCl₃) δ 167.59, 145.54, 145.37, 129.82, 128.43, 127.28, 118.34, 113.41, 113.28, 92.45, 38.96, 33.96, 22.92, 22.06, 21.08.

### Synthesis of N-(3-cyano-4,5,6,7-tetrahydrobenzo[b]thiophen-2-yl)-3-(prop-2-yn-1-ylamino) propanamide (PKL-A69)

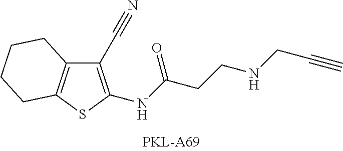 [SMILES: C#CCNCCC(=O)Nc1sc2c(c1C#N)CCCC2]

Following general procedure for the synthesis of Michael adducts, N-(3-cyano-4,5,6,7-tetrahydrobenzo[b]thiophen-2-yl) acrylamide (PKL-A67) reacted with prop-2-yn-1-amine to afford (82%) of the title compound as a white solid after column chromatography (EtOAc in Hexane: 0-75%).

¹H NMR (500 MHz, CDCl₃) δ 12.54 (s, 1H), 3.63 (d, J=2.4 Hz, 2H), 3.15-3.11 (m, 2H), 2.64-2.55 (m, 7H), 2.29 (t, J=2.4 Hz, 1H), 1.81 (tdd, J=2.3, 3.8, 8.8 Hz, 5H). ¹³C NMR (126 MHz, CDCl₃) δ 168.30, 146.38, 129.67, 126.68, 113.73, 92.16, 79.21, 71.85, 42.09, 36.09, 32.87, 22.99, 22.96, 22.12, 21.16.

### Synthesis of N-(3-cyano-4,5,6,7-tetrahydrobenzo[b]thiophen-2-yl)-3-((2-(pyridin-3-yl)ethyl)amino)propanamide (PKL-A68)

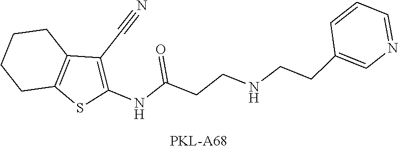 [SMILES: N#Cc1c(NC(=O)CCNCCc2cccnc2)sc2c1CCCC2]

Following general procedure for the Synthesis of Michael adducts, N-(3-cyano-4,5,6,7-tetrahydrobenzo[b]thiophen-2-yl) acrylamide (PKL-A67) reacted with 2-(pyridin-3-yl)ethan-1-amine to afford (72%) of the title compound as a white solid after column chromatography (EtOAc in Hexane: 20-95%).

¹H NMR (500 MHz, DMSO) δ 8.47 (s, 1H), 8.41 (d, J=4.8 Hz, 1H), 7.67 (d, J=7.8 Hz, 1H), 7.31 (dd, J=4.8, 7.8 Hz, 1H), 3.17 (s, 2H), 3.00-2.90 (m, 4H), 2.86 (t, J=7.4 Hz, 2H), 2.63 (t, J=5.5 Hz, 2H), 2.57 (d, J=4.9 Hz, 2H), 1.75 (s, 4H). ¹³C NMR (126 MHz, DMSO) δ 170.36, 150.28, 148.29, 147.96, 136.63, 135.28, 130.75, 127.10, 123.92, 115.15, 92.44, 79.70, 79.43, 79-17, 23.95, 23.84, 23.13, 22.23.

### Synthesis of methyl (3-((3-cyano-4,5,6,7-tetrahydrobenzo[b]thiophen-2-yl)amino)-3-oxopropyl) glycinate (PKL-A72)

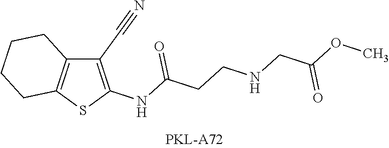 [SMILES: COC(=O)CNCCC(=O)Nc1sc2c(c1C#N)CCCC2]

Following general procedure for the Synthesis of Michael adducts, N-(3-cyano-4,5,6,7-tetrahydrobenzo[b]thiophen-2-yl) acrylamide (PKL-A67) reacted with methyl glycinate to afford (75%) of the title compound as a white solid after column chromatography (EtOAc in Hexane: 0-85%).

¹H NMR (500 MHz, CDCl₃) δ 12.64 (s, 1H), 3.76 (s, 3H), 3.59 (s, 2H), 3.07-3.01 (m, 2H), 2.62 (td, J=2.9, 5.0, 5.8 Hz, 2H), 2.58-2.54 (m, 4H), 1.81 (ddq, J=2.5, 3.0, 4.6, 6.8 Hz, 4H). ¹³C NMR (126 MHz, CDCl₃) δ 170.96, 168.23, 146.33, 129.68, 126.64, 113.76, 92.19, 51.10, 48.46, 43.49, 33.46, 22.99, 22.96, 22.13, 21.17.

### Synthesis of N-(3-cyano-4,5,6,7-tetrahydrobenzo[b]thiophen-2-yl)-3-((2-phenoxyethyl)amino) propanamide (PKL-A95)

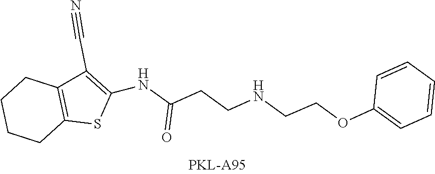 [SMILES: N#Cc1c(NC(=O)CCNCCOc2ccccc2)sc2c1CCCC2]

Following general procedure for the Synthesis of Michael adducts, N-(3-cyano-4,5,6,7-tetrahydrobenzo[b]thiophen-2-yl) acrylamide (PKL-A67) reacted with 2-phenoxyethan-1-amine to afford (74%) of the title compound as a white solid after column chromatography (EtOAc in Hexane: 0-85%).

¹H NMR (500 MHz, CDCl₃) δ 12.86 (s, 1H), 7.22 (d, J=7.7 Hz, 2H), 6.87 (dd, J=7.9, 16.8 Hz, 3H), 4.21 (t, J=4.9 Hz, 2H), 3.08 (t, J=4.9 Hz, 2H), 3.01 (t, J=5.7 Hz, 2H), 2.54-2.44 (m, 5H), 1.73 (q, J=7.2 Hz, 4H). ¹³C NMR (126 MHz, CDCl₃) δ 168.61, 157.52, 146.46, 129.67, 128.48, 126.58, 119.98, 113.98, 113.49, 92.03, 65.35, 47.24, 43.62, 33.38, 22.96, 22.93, 22.12, 21.18.

### Synthesis of N-(3-cyano-4,5,6,7-tetrahydrobenzo[b]thiophen-2-yl)-3,4-dimethoxybenzamide (PKL-A79)

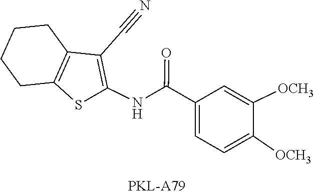 [SMILES: COc1ccc(C(=O)Nc2sc3c(c2C#N)CCCC3)cc1OC]

Following general procedure A for the synthesis of PKL-A79, 2-amino-4,5,6,7-tetrahydrobenzo[b]thiophene-3-carbonitrile (JNK-1) reacted with Dimethoxy Benzoyl chloride to afford (70%) of the title compound as a white solid after column chromatography (EtOAc in Hexane: 0-40%). ¹H NMR (500 MHz, CDCl₃) δ 8.78 (s, 1H), 7.55-7.50 (m, 1H), 7.44 (dt, J=1.4, 8.5 Hz, 1H), 6.95 (d, J=8.3 Hz, 1H), 3.97 (d, J=2.5 Hz, 6H), 2.67 (t, J=5.6 Hz, 2H), 2.63 (t, J=5.7 Hz, 2H), 1.85 (q, J=7.0 Hz, 4H). ¹³C NMR (126 MHz, CDCl₃) δ 162.87, 153.02, 149.42, 146.91, 130.82, 128.43, 124.13, 119.78, 114.49, 110.76, 110.40, 77.16, 56.08, 56.03, 53.32, 24.40, 24.01, 23.88.

### Synthesis of N-(3-cyano-4,5,6,7-tetrahydrobenzo[b]thiophen-2-yl)-2-phenoxyacetamide (PKL-A78)

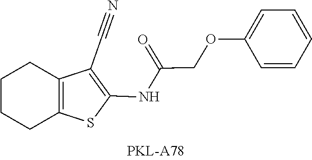 [SMILES: N#Cc1c(NC(=O)COc2ccccc2)sc2c1CCCC2]

Following general procedure B, To a solution of 2-phenoxyacetic acid (1 equiv.), EDCl (1.2 equiv.) and HOBt (1.2 equiv.) in DMF (0.1-0.3 M) Triethylamine (1.5 equiv.) were added at 0° C. The mixture was stirred for 30 min at room temperature. Then, 2-amino-4,5,6,7-tetrahydrobenzo[b]thiophene-3-carbonitrile (JNK-1) (1.2 equiv.) was added, and the reaction mixture was stirred at room temperature for 36 h, to afford 65% of the title compound as white solid after column chromatography (EtOAc in Hexane: 0-40%).

¹H NMR (500 MHz, CDCl₃) δ 9.34 (s, 1H), 7.36 (t, J=7.8 Hz, 2H), 7.08 (t, J=7.4 Hz, 1H), 7.02 (d, J=8.1 Hz, 2H), 4.72 (s, 2H), 2.64 (dt, J=5.5, 21.1 Hz, 4H), 1.84 (q, J=7.4 Hz, 5H). ¹³C NMR (126 MHz, CDCl₃) δ 165.32, 156.91, 145.32, 131.65, 130.40, 129.89, 129.41, 123.25, 115.28, 114.23, 95.31, 77.16, 67.17, 24.43, 24.39, 23.46, 22.47.

### Synthesis of N-(3-cyano-4,5,6,7-tetrahydrobenzo[b]thiophen-2-yl)-3-hydroxyisonicotinamide (PKL-A102)

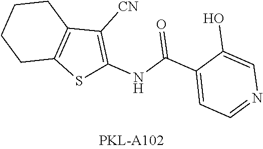 [SMILES: N#Cc1c(NC(=O)c2ccncc2O)sc2c1CCCC2]

Following general procedure B, To a solution of 3-hydroxyisonicotinic acid (1 equiv.), EDCl (1.2 equiv.) and HOBt (1.2 equiv.) in DMF (0.1-0.3 M) Triethylamine (1.5 equiv.) were added at 0° C. The mixture was stirred for 30 min at room temperature. Then, 2-amino-4,5,6,7-tetrahydrobenzo[b]thiophene-3-carbonitrile (JNK-1) (1.2 equiv.) was added, and the reaction mixture was stirred at room temperature for 36 h, to afford 52% of the title compound as a white solid after column chromatography (EtOAc in Hexane: 0-95%).

¹H NMR (500 MHz, DMSO) δ 11.74 (s, 1H), 7.29 (s, 1H), 7.07 (d, J=5.0 Hz, 1H), 6.82 (d, J=4.9 Hz, 1H), 1.77-1.67 (m, 4H), 0.91 (dq, J=2.0, 3.1, 7.0 Hz, 4H). ¹³C NMR (126 MHz, DMSO) δ 165.61, 157.97, 140-47, 136.55, 129.91, 126.13, 125.37, 122.67, 121.13, 117.02, 92.42, 39.52, 24.33, 24.14, 23.49, 22.61.

### Synthesis of N-(3-cyano-4,5,6,7-tetrahydrobenzo[b]thiophen-2-yl) nicotinamide (PKL-A103)

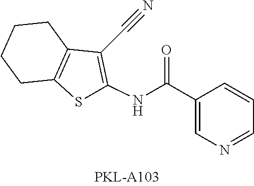 [SMILES: N#Cc1c(NC(=O)c2cccnc2)sc2c1CCCC2]

Following general procedure B, to a solution of nicotinic acid (1 equiv.), EDCl (1.2 equiv.) and HOBt (1.2 equiv.) in DMF (0.1-0.3 M) Triethylamine (1.5 equiv.) were added at 0° C. The mixture was stirred for 30 min at room temperature. Then, 2-amino-4,5,6,7-tetrahydrobenzo[b]thiophene-3-carbonitrile (JNK-1) (1.2 equiv.) was added, and the reaction mixture was stirred at room temperature for 48 h, to afford 45% of the title compound as a white solid after column chromatography (EtOAc in Hexane: 0-85%).

¹H NMR (500 MHz, DMSO) δ 11.17 (s, 1H), 8.25 (d, J=2.3 Hz, 1H), 7.97 (dd, J=1.9, 4.8 Hz, 1H), 7.46 (dt, J=2.1, 7.9 Hz, 1H), 6.77 (dd, J=4.9, 7.9 Hz, 1H), 1.76-1.71 (m, 4H), 1.02-0.92 (m, 4H). ¹³C NMR (126 MHz, DMSO) δ 164.41, 153.26, 149.62, 136.59, 131.98, 129.28, 128.93, 123.97, 114.59, 96.42, 29.49, 24.09, 23.93, 23.06, 22.17.

### Synthesis of N-(3-cyano-4,5,6,7-tetrahydrobenzo[b]thiophen-2-yl) pyridine-3-sulfonamide (PKL-A82)

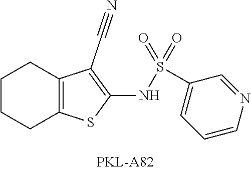 [SMILES: N#Cc1c(NS(=O)(=O)c2cccnc2)sc2c1CCCC2]

Following general procedure A for the synthesis of PKL-A82, 2-amino-4,5,6,7-tetrahydrobenzo[b]thiophene-3-carbonitrile (JNK-1) reacted with pyridine-3-sulfonyl chloride to afford (70%) of the title compound as a white solid after column chromatography (EtOAc in Hexane: 0-40%).

¹H NMR (500 MHz, CDCl₃) δ 9.03 (d, J=2.2 Hz, 1H), 8.87 (d, J=4.9 Hz, 1H), 8.21-8.17 (m, 1H), 7.52 (dd, J=4.8, 8.1 Hz, 1H), 5.30 (s, 1H), 2.65 (t, J=6.1 Hz, 2H), 2.51 (t, J=6.1 Hz, 2H), 1.86-1.76 (m, 4H). ¹³C NMR (126 MHz, CDCl₃) δ 153.26, 147.31, 142.07, 134.17, 133.92, 133.06, 132.73, 123.14, 111.90, 103.11, 23.46, 23.12, 21.76, 20.74.

A schematic view of the synthesis of analogues of aminothiophene can be seen in FIG. 8 and the synthesis procedure for the specific compounds follows below.

### Synthesis of 2-chloro-N-(3-cyano-4,5,6,7-tetrahydrobenzo[b]thiophen-2-yl)acetamide (PKL-B83)

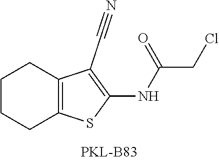 [SMILES: N#Cc1c(NC(=O)CCl)sc2c1CCCC2]

To solution of aminothiophene JNK-1 (200 mg, 1.12 mmol) in DMF, Triethylamine (0.17 mL, 1.23 mmol) was added dropwise to the reaction mixture at 0° C. The mixture was stirred for 30 min at room temperature. Then chloro acetyl chloride (0.1 mL, 1.23 mmol) was added, and the reaction mixture was stirred at room temperature for 1h. The residue was poured to ice, and then extracted with Chloroform. The combined organic extracts were washed with brine, dried over magnesium sulphate and evaporated to dryness under reduced pressure to afford 230 mg (81%) of the desired product as a brown solid.

¹H NMR (500 MHz, CDCl₃) δ 9.22 (s, 1H), 4.21 (s, 2H), 2.59 (ddd, J=7.8, 5.3, 1.8 Hz, 2H), 2.54 (td, J=6.0, 1.8 Hz, 2H), 1.81-1.73 (m, 5H). ¹³C NMR (126 MHz, CDCl₃) δ 162.80, 145.03, 131.40, 129.46, 113.79, 95.15, 42.03, 24.03, 23.98, 23.03, 22.04.

### Synthesis of Representative Molecule PKL-B78

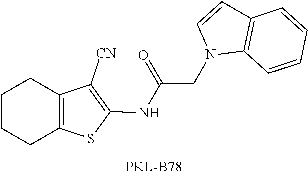 [SMILES: N#Cc1c(NC(=O)Cn2ccc3ccccc32)sc2c1CCCC2]

Following general procedure D, PKL-B83 (80 mg, 0.31 mmol), was reacted with indole (36.79 mg, 0.31 mmol), to afford the title compound as a white solid after column chromatography (EtOAc in Hexane: 0-40%).’

¹H NMR (500 MHz, CDCl₃) δ 8.04 (s, 1H), 7.64 (dt, J=7.9, 1.0 Hz, 1H), 7.25-7.20 (m, 2H), 7.13 (ddd, J=8.0, 6.5, 1.5 Hz, 1H), 7.10 (d, J=3.3 Hz, 1H), 6.66 (dd, J=3.2, 0.8 Hz, 1H), 4.95 (s, 2H), 2.55-2.51 (m, 2H), 2.42 (d, J=1.8 Hz, 2H), 1.70 (ddddd, J=15.3, 7.8, 6.0, 3.6, 1.7 Hz, 4H). ¹³C NMR (126 MHz, CDCl₃) δ 165.43, 144.93, 136.15, 131.24, 129.20, 129.05, 127.68, 123.14, 121.88, 120.94, 113.23, 108.87, 104.85, 95.15, 49.55, 23.99, 23.91, 23.00, 21.99.

### Synthesis of Representative Molecule PKL-B125i

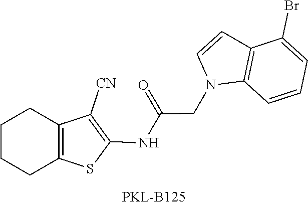 [SMILES: N#Cc1c(NC(=O)Cn2ccc3c(Br)cccc32)sc2c1CCCC2]

A solution of 4-Bromoindole (0.64 mL, 0.51 mmol), potassium hydroxide (34.34 mg, 0.61 mmol) and TBAB (Catalytic amount 0.02 equiv.) in THF was stirred for 30 min at room temperature. Then PKL-B83 (129.9 mg, 0.51 mmol) was added, and the reaction mixture was stirred at room temperature for 18 h. The residue was filtered and evaporated. The solid obtained was washed with water and then ethanol, to afford the desired product as a white solid.

¹H NMR (500 MHz, DMSO) δ 12.08 (s, 1H), 7.58 (d, J=3.2 Hz, 1H), 7.52 (d, J=8.2 Hz, 1H), 7.32 (d, J=7.5 Hz, 1H), 7.13 (t, J=7.9 Hz, 1H), 6.50 (dd, J=3.2, 0.8 Hz, 1H), 5.34 (s, 2H), 2.61 (tt, J=4.9, 2.3 Hz, 4H), 1.80 (p, J=2.9 Hz, 4H). ¹³C NMR (126 MHz, DMSO) δ 166.75, 137.43, 131.57, 131.26, 128.85, 127.76, 122.95, 122.44, 114.84, 113.99, 110.12, 101.31, 93.37, 49.15, 23.99, 23.80, 23.06, 22.17.

### Synthesis of tert-butyl (3-cyano-4,5,6,7-tetrahydrobenzo[b]thiophen-2-yl)carbamate (PKL-B61)

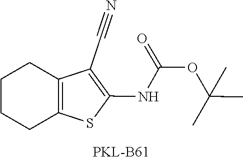 [SMILES: CC(C)(C)OC(=O)Nc1sc2c(c1C#N)CCCC2]

Di-tert-butyl decarbonate (673.41 mg; 3.09 mmol) was added to a stirred solution of aminothiophene (500 mg, 3.09 mmol) and Triethylamine 0.4 mL; 3.37) in THF/H₂O (4:1). The mixture was stirred for 16 hours at room temperature. Concentrate then add H2O then acidified the mixture to PH=2 with HCl (1M). Aqeuous layer was extracted with EtOAc (×4) and combined and washed with saturated NaHCO₃ and Brine, dry using NaSO₄, Filter and concentrate.

¹H NMR (500 MHz, CDCl₃) δ 7.38 (s, 1H), 2.57-2.51 (m, 2H), 2.49 (t, J=5.7 Hz, 2H), 1.75 (hept, J=3.4 Hz, 4H), 1.46 (s, 9H). ¹³C NMR (126 MHz, CDCl₃) δ 150.29, 147.56, 129.99, 125.94, 113.49, 82.02, 27.12, 22.97, 22.89, 22.10, 21.08.

### Synthesis of tert-butyl (3-cyano-4,5,6,7-tetrahydrobenzo[b]thiophen-2-yl)(prop-2-yn-1-yl) carbamate (PKL-B57)

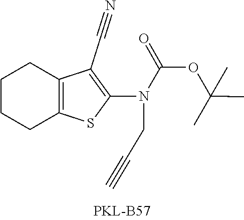 [SMILES: C#CCN(C(=O)OC(C)(C)C)c1sc2c(c1C#N)CCCC2]

A solution of PKL-B61 (100 mg, 0.36 mmol), potassium carbonate (59.58 mg, 0.43 mmol) and TBAB (Catalytic amount 0.2 equiv.) in THF was stirred for 30 min at room temperature. Then propargyl bromide solution (0.032 ml, 0.43 mmol) was added, and the reaction mixture was stirred at room temperature for 48 h. The residue was partitioned between water and ethyl acetate and the aqueous extracted with further ethyl acetate. The combined organic extracts were washed with brine, dried over magnesium sulphate and evaporated to dryness under reduced pressure to give the desired product as a yellow liquid.

¹H NMR (500 MHz, CDCl₃) δ 4.36 (d, J=2.4 Hz, 2H), 2.61 (t, J=5.8 Hz, 2H), 2.56 (t, J=5.8 Hz, 2H), 2.25 (d, J=2.6 Hz, 1H), 1.78 (q, J=7.3, 6.4 Hz, 4H), 1.42 (s, 9H). ¹³C NMR (126 MHz, CDCl₃) δ 152.61, 149.48, 134.26, 133.67, 113.65, 83.15, 78.25, 73.51, 28.04, 24.68, 24.31, 22.97, 22.00.

### Synthesis of ethyl (3-cyano-4,5,6,7-tetrahydrobenzo[b]thiophen-2-yl)glycinate (PKL-B54)

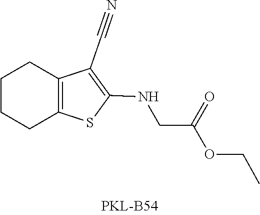 [SMILES: CCOC(=O)CNc1sc2c(c1C#N)CCCC2]

A solution of aminothiophene JNK-1 (100 mg, 0.39 mmol), potassium hydroxide (37.77 mg, 0.43 mmol) and TBAB (Catalytic amount 0.2 equiv.) in Acetone/H₂O (4:1) was stirred for 30 min at room temperature. Then Bromo ethyl acetate (68 μl, 0.43 mmol) was added, and the reaction mixture was stirred at 60° C. for 18 h. The residue was partitioned between water and ethyl acetate and the aqueous extracted with further ethyl acetate. The combined organic extracts were washed with brine, dried over magnesium sulphate and evaporated to dryness under reduced pressure, mounted on silica and purified by flash chromatography to afford the desired product as a white solid.

¹H NMR (500 MHz, CDCl₃) δ 5.17 (s, 1H), 4.20 (q, J=7.1 Hz, 2H), 3.91 (s, 2H), 2.44 (q, J=5.3 Hz, 4H), 1.72 (ddtd, J=14.0, 8.6, 6.1, 2.3 Hz, 4H), 1.24 (t, J=7.2 Hz, 3H). 13C NMR (126 MHz, CDCl3) δ 169.15, 161.23, 133.23, 119.80, 115.74, 86.26, 61.92, 48.39, 24.47, 24.16, 23.34, 22.10, 14.15.

### Synthesis of (3-cyano-4,5,6,7-tetrahydrobenzo[b]thiophen-2-yl)glycine (PKL-B55)

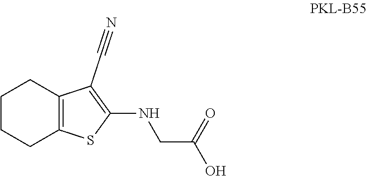 [SMILES: N#Cc1c(NCC(=O)O)sc2c1CCCC2]

To a solution of PKL-B54 (200 mg; 0.75 mmol) in THF/H₂O (2:1) was added LiOH,H₂O (47.62 mg, 1.13 mmol) and the reaction mixture was stirred at room temperature for 1h. The reaction was quenched with 0.5M aqueous solution of HCl and extracted with EtOAc (20 mL×3). The combined organic layers were washed with brine, dried over MgSO₄ and filtered. The solvents were removed under reduced pressure to yield the desired acid.

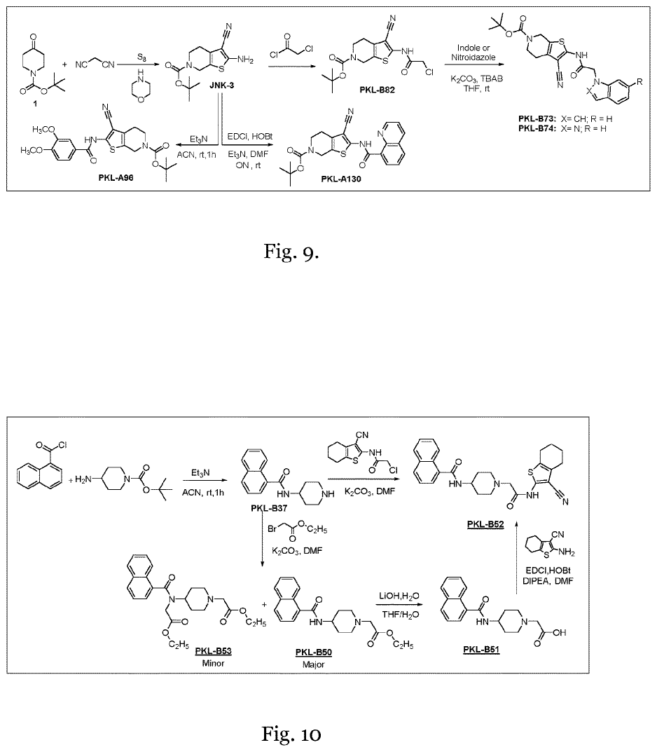

A schematic view of the synthesis of analogues of N-(3-cyano-4,5,6,7-tetrahydrobenzo[b]thiophen-2-yl)-1-naphthamide with a linker PKL-B52 can be seen in FIG. 9 and the synthesis procedure for the specific compounds follows below.

### Synthesis of N-(piperidin-4-yl)-1-naphthamide (PKL-B37)

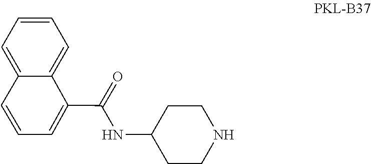 [SMILES: O=C(NC1CCNCC1)c1cccc2ccccc12]

A solution of tert-butyl 4-aminopiperidine-1-carboxylate (1 g, 4.99 mmol), and naphtaloyl chloride (0.1 mL, 5.49 mmol) in Acetonitrile was stirred for 2 h at 80° C. Then reaction mixture was cooled and filtered, the reaction was monitored by TLC and LCMSMS, the compound was directly deprotected during the reaction. The liquid part was evaporated to dryness under reduced pressure to give the desired product as a white solid.

### Synthesis of ethyl 2-(4-(1-naphthamido)piperidin-1-yl)acetate PKL-B50 and ethyl 2-(4-(N-(2-ethoxy-2-oxoethyl)-1-naphthamido))piperidin-1-yl)acetate (PKL-B53)

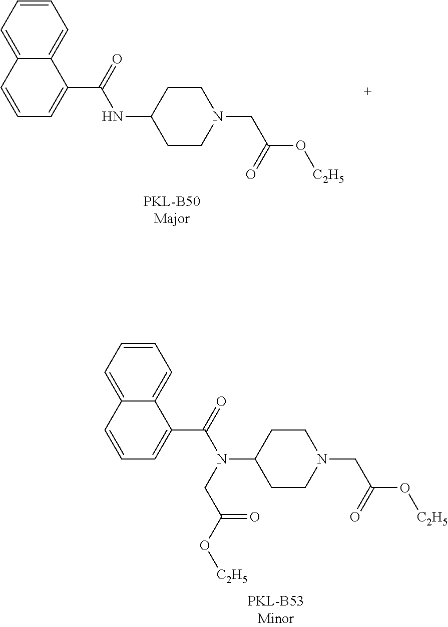 [SMILES: CCOC(=O)CN1CCC(NC(=O)c2cccc3ccccc23)CC1 ; CCOC(=O)CN1CCC(N(CC(=O)OCC)C(=O)c2cccc3ccccc23)CC1]

A solution of PKL-B37 (100 mg, 0.39 mmol), potassium carbonate (59.77 mg, 0.43 mmol) and TBAB (Catalytic amount 0.2 equiv.) in DMF was stirred for 30 min at 60° C. Then Bromo ethyl acetate (47 μl, 0.43 mmol) was added, and the reaction mixture was stirred at room temperature for 48 h. The residue was partitioned between water and ethyl acetate and the aqueous extracted with further ethyl acetate. The combined organic extracts were washed with brine, dried over magnesium sulphate and evaporated to dryness under reduced pressure, mounted on silica and purified by flash chromatography to afford the desired products both as transparent liquids.

### Synthesis of 2-(4-(1-naphthamido)piperidin-1-yl)acetic acid (PKL-B51)

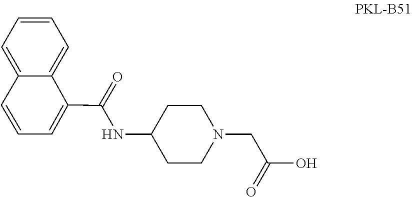 [SMILES: O=C(O)CN1CCC(NC(=O)c2cccc3ccccc23)CC1]

To a solution of PKL-B50 (200 mg; 0.58 mmol) in THF/H₂O (2:1) was added LiOH,H₂o (29.58 mg, 0.70 mmol) and the reaction mixture was stirred at room temperature for 30 min. The reaction was quenched with 0.5M aqueous solution of HCl and extracted with EtOAc (20 mL×3). The combined organic layers were washed with brine, dried over MgSO₄ and filtered. The solvents were removed under reduced pressure to yield the desired acid as a white solid.

### Synthesis of N-(1-(2-((3-cyano-4,5,6,7-tetrahydrobenzo[b]thiophen-2-yl)amino)-2-oxoethyl)piperidin-4-yl)-1-naphthamide (PKL-B52)

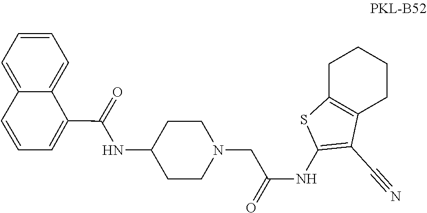 [SMILES: N#Cc1c(NC(=O)CN2CCC(NC(=O)c3cccc4ccccc34)CC2)sc2c1CCCC2]

This compound was prepared using two different methods.

Method 1: Following general procedure D, PKL-B82 (60.09 mg, 0.25 mmol), was reacted with PKL-B37 (60 mg, 0.23 mmol), to afford 70 mg (63%) of the title compound as a white solid after column chromatography (EtOAc in Hexane: 0-50%).

Method 2: To solution of PKL-B51 (60 mg, 0.19 mmol), EDCl (65.74 mg, 0.19 mmol) and HOBt (38.93 mg, 0.45 mmol) in DMF, triethyl amine (40.16 μL, 0.29 mmol) was added at 0° C. The mixture was stirred for 30 min at room temperature. Then aminothiophene (34.24 mg, 0.19 mmol) was added, and the reaction mixture was stirred at room temperature for 16 h. The residue was poured to ice, and then extracted with ethyl acetate. The combined organic extracts were washed with brine, dried over magnesium sulphate and evaporated to dryness under reduced pressure to afford 76 mg (84%) the desired product as a white solid.

¹H NMR (500 MHz, CDCl₃) δ 10.40 (s, 1H), 8.23-8.20 (m, 1H), 7.84 (dt, J=8.3, 1.1 Hz, 1H), 7.81-7.78 (m, 1H), 7.53 (dd, J=7.0, 1.2 Hz, 1H), 7.51-7.43 (m, 2H), 7.39 (dd, J=8.2, 7.0 Hz, 1H), 3.18 (s, 2H), 2.91-2.83 (m, 2H), 2.57 (td, J=5.9, 1.9 Hz, 2H), 2.50 (ddd, J=12.7, 8.3, 6.3 Hz, 4H), 2.14 (dd, J=13.0, 3.8 Hz, 2H), 1.79-1.71 (m, 5H), 1.68 (dd, J=12.0, 3.5 Hz, 2H). ¹³C NMR (126 MHz, CDCl₃) δ 169.12, 167.20, 146.47, 134-37, 133.71, 130.77, 130.66, 130.10, 128.35, 128.33, 127.16, 126.44, 125.28, 124.95, 124.73, 114.34, 93.79, 60.55, 53.07, 46.33, 32.64, 31.60, 24.09, 24.02, 23.11, 22.11.

A schematic view of the synthesis of amide analogues of 2-amino-5,6,7,8-tetrahydro-4H-cyclohepta[b]thiophene-3-carbonitrile can be seen in FIG. 6 and the synthesis procedure for the specific compounds follows below.

### Synthesis of 2-amino-5,6,7,8-tetrahydro-4H-cyclohepta[b]thiophene-3-carbonitrile (SI-44)

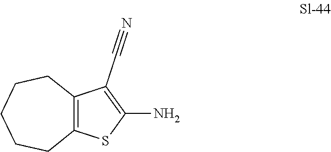 [SMILES: N#Cc1c(N)sc2c1CCCCC2]

The title compound, was synthesized by preparing a mixture of cycloheptanone (1.0 g, 1 equiv.), malononitrile (0.66 g, 0.63 ml, 1 equiv.), sulphur Ss (0.32 g, 1 equiv.) and ethanol (5 ml). The obtained mixture was stirred at 45-50° C. Once the temperature was obtained, diethylamine (1.02 ml) was added dropwise until the sulphur completely dissolved. The obtained solid was filtered, washed with ethanol and recrystallized from benzene. Yield 61%; m.p. 125-126° C. (lit.21 125° C.).

¹H NMR (500 MHz, CDCl₃) δ 4.49 (s, 2H, NH₂), 2.63-2.59 (m, 4H), 1.85-1.81 (m, 2H), 1.68-1.63 (m, 4H).

### General Procedure for the Synthesis of Amide Analogues of 2-amino-5,6,7,8-tetrahydro-4H-cyclohepta[b]thiophene-3-carbonitrile (1-2)

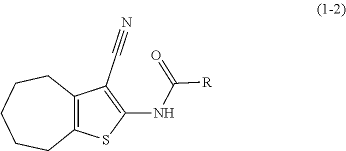

An oven-dried 100 mL round-bottom flask was charged with intermediate compound SI-44 (1 equiv.), dimethoxy benzoyl chloride/1-naphthoyl chloride (1.2 equiv.) and pyridine as solvent and stirred under an N₂ atmosphere for 12h. Reaction was monitored by TLC analysis. The solvent was later removed under reduced pressure. Purification was performed by Silica column chromatography to afford desired product.

### Synthesis of N-(3-cyano-5,6,7,8-tetrahydro-4H-cyclohepta[b]thiophen-2-yl)-1-naphthamide (PKL-A56)

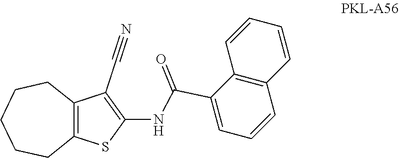 [SMILES: N#Cc1c(NC(=O)c2cccc3ccccc23)sc2c1CCCCC2]

Following general procedure A for the synthesis of PKL-A56, 2-amino-5,6,7,8-tetrahydro-4H-cyclohepta[b]thiophene-3-carbonitrile (SI-44) reacted with 1-naphthoyl chloride to afford (78%) of the title compound as a white crystal solid after column chromatography (EtOAc in Hexane: 0-30%).

¹H NMR (500 MHz, CDCl₃) δ 8.69 (s, 1H), 8.40 (d, J=8.3 Hz, 1H), 8.05 (d, J=8.2 Hz, 1H), 7.93 (d, J=7.9 Hz, 1H), 7.80 (d, J=7.1 Hz, 1H), 7.65-7.52 (m, 3H), 2.78 (td, J=5.1, 8.6 Hz, 4H), 1.90 (p, J=5.6 Hz, 2H), 1.71 (dp, J=5.5, 16.4 Hz, 4H). ¹³C NMR (126 MHz, CDCl₃) δ 164.33, 143.21, 134.91, 132.82, 131.50, 131.43, 129.96, 129.20, 127.60, 126.92, 125.94, 124.97, 123.97, 123.64, 113.74, 95.63, 31.03, 28.20, 28.08, 27.04, 26.33.

### Synthesis of N-(3-cyano-5,6,7,8-tetrahydro-4H-cyclohepta[b]thiophen-2-yl)-3,4-dimethoxy-benzamide (PKL-A135)

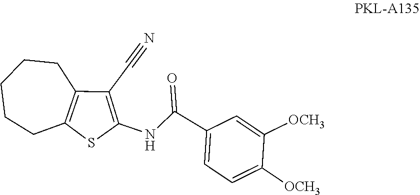 [SMILES: COc1ccc(C(=O)Nc2sc3c(c2C#N)CCCCC3)cc1OC]

Following general procedure A for the synthesis of PKL-A135, 2-amino-5,6,7,8-tetrahydro-4H-cyclohepta[b]thiophene-3-carbonitrile (SI-44) reacted with Dimethoxy Benzoyl chloride to afford (71%) of the title compound as a white solid after column chromatography (EtOAc in Hexane: 0-40%).

¹H NMR (500 MHz, CDCl₃) δ 8.72 (s, 1H), 7.52 (d, J=2.1 Hz, 1H), 7.43 (dd, J=2.1, 8.4 Hz, 1H), 6.94 (d, J=8.4 Hz, 1H), 3.97 (d, J=2.6 Hz, 6H), 2.79-2.73 (m, 4H), 1.88 (dq, J=5.7, 8.8 Hz, 2H), 1.73-1.65 (m, 4H). ¹³C NMR (126 MHz, CDCl₃) δ 163.01, 153-11, 149-54, 144.78, 135.73, 132.18, 124.25, 119.84, 115.03, 110.85, 110.53, 96.18, 56.20, 56.14, 32.04, 29.18, 29.13, 28.05, 27.35.

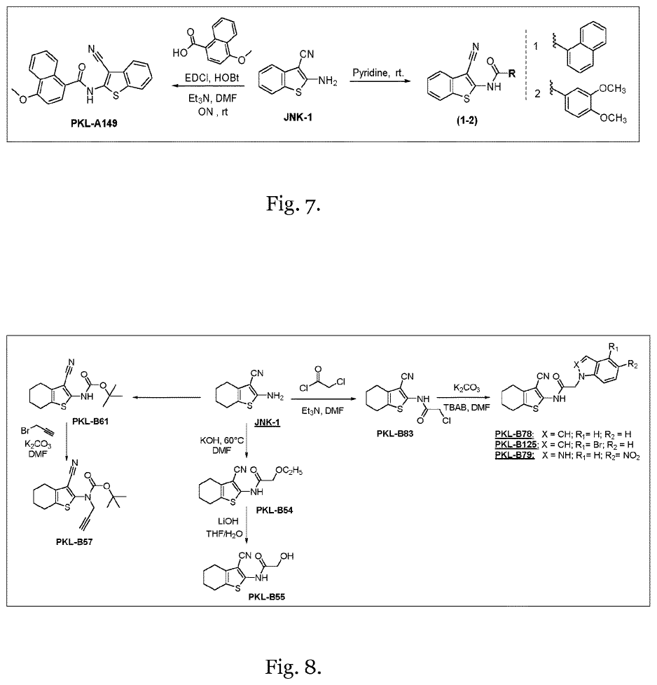

A schematic view of the synthesis of amide analogues of 2-aminobenzo[b]thiophene-3-carbonitrile can be seen in FIG. 7 and the synthesis procedure for the specific compounds follows below.

### Synthesis of N-(3-cyanobenzo[b]thiophen-2-yl)-4-methoxy-1-naphthamide (PKL-A140)

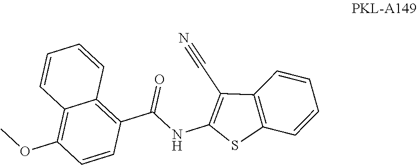 [SMILES: COc1ccc(C(=O)Nc2sc3ccccc3c2C#N)c2ccccc12]

Following general procedure B, to a solution of carboxylic acid (1 equiv.), EDCl (1.2 equiv.) and HOBt (1.2 equiv.) in DMF (0.1-0.3 M), triethylamine (1.5 equiv.) were added at 0° C. The mixture was stirred for 30 min at room temperature. Then, 2-aminobenzo[b]thiophene-3-carbonitrile (1.2 equiv.) was added, and the reaction mixture was stirred at room temperature for 6-16 h, to afford 72% of the title compound as a white solid after column chromatography (EtOAc in Hexane: 0-20%).

¹H NMR (500 MHz, DMSO) δ 11.74 (s, 1H), 7.52 (d, J=8.5 Hz, 1H), 7.47 (dd, J=1.3, 8.4 Hz, 1H), 7.24 (d, J=8.0 Hz, 1H), 7.15 (d, J=8.1 Hz, 1H), 6.91 (d, J=7.9 Hz, 1H), 6.86 (ddd, J=1.5, 6.8, 8.5 Hz, 1H), 6.79 (ddd, J=1.3, 6.8, 8.1 Hz, 1H), 6.73 (td, J=1.1, 7.6 Hz, 1H), 6.63 (td, J=1.2, 7.2, 7.7 Hz, 1H), 6.33 (d, J=8.2 Hz, 1H), 3.26 (s, 3H). ¹³C NMR (126 MHz, DMSO) δ 167.73, 158.11, 134.87, 133.39, 131.73, 129.99, 128.51, 126.68, 126.46, 125.33, 125.21, 123.28, 122.44, 120.38, 114.33, 103.70, 88.79, 56.65.

### General Procedure for the Synthesis of Amide Analogues of 2-aminobenzo[b]thiophene-3-carbonitrile (1-2)

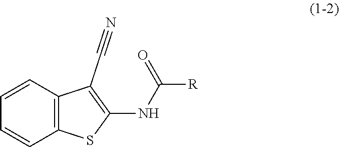

An oven-dried 100 mL round-bottom flask was charged with 2-aminobenzo[b]thiophene-3-carbonitrile (1 equiv.), Dimethoxy Benzoyl chloride/1-Naphthoyl chloride (1.2 equiv.) and pyridine as solvent, stir under an N₂ atmosphere for 3h. Reaction was monitored by TLC analysis. Later solvent was removed under reduced pressure. Purification was performed by (hand column) Silica column chromatography to afford desired product.

### Synthesis of N-(3-cyanobenzo[b]thiophen-2-yl)-1-naphthamide (PKL-A31)

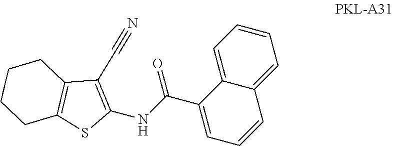 [SMILES: N#Cc1c(NC(=O)c2cccc3ccccc23)sc2c1CCCC2]

Following general procedure for the synthesis of amide analogues of 2-aminobenzo[b]thiophene-3-carbonitrile, 90% of the title compound was afforded as a white solid after column chromatography (EtOAc in Hexane: 0-40%). ¹H NMR (500 MHz, CDCl₃) δ 9.04 (s, 1H), 8.49-8.44 (m, 1H), 8.10 (d, J=8.3 Hz, 1H), 7.98-7.94 (m, 1H), 7.90 (dd, J=1.2, 7.2 Hz, 1H), 7.86 (d, J=8.0 Hz, 1H), 7.83-7.79 (m, 1H), 7.69-7.63 (m, 1H), 7.60 (dd, J=7.2, 8.3 Hz, 2H), 7.54-7.50 (m, 1H), 7.43 (ddd, J=1.2, 7.3, 8.3 Hz, 1H). ¹³C NMR (126 MHz, CDCl3) δ 165.92, 150.79, 133.61, 133.15, 128.78, 128.26, 127.16, 126.36, 126.28, 125.22, 124.90, 124.68, 122.42, 120.90, 113.92.

### Synthesis of N-(3-cyanobenzo[b]thiophen-2-yl)-3,4-dimethoxybenzamide (PKL-A142)

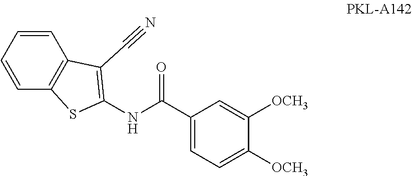 [SMILES: COc1ccc(C(=O)Nc2sc3ccccc3c2C#N)cc1OC]

Following general procedure for the synthesis of amide analogues of 2-aminobenzo[b]thiophene-3-carbonitrile, afford 85% of the title compound as a white solid after column chromatography (EtOAc in Hexane: 0-40%). ¹H NMR (500 MHz, CDCl₃) δ 9.01 (s, 1H), 7.73 (ddt, J=0.9, 7.9, 12.8 Hz, 2H), 7.51 (d, J=2.2 Hz, 1H), 7.48-7.40 (m, 2H), 7.33 (ddd, J=1.2, 7.2, 8.3 Hz, 1H), 6.92 (d, J=8.4 Hz, 1H), 3.92 (d, J=1.4 Hz, 6H). ¹³C NMR (126 MHz, CDCl₃) δ 163.58, 151.36, 149.68, 133.73, 133.59, 126.20, 125.03, 123.68, 122.37, 120.75, 120.30, 114-19, 110.99, 110.61, 88.50, 56.26, 56.20.

A schematic view of the synthesis of analogues of tert-butyl 2-amino-3-cyano-4,7-dihydrothieno[2,3-c]pyridine-6(5H)-carboxylate can be seen in FIG. 10 and the synthesis procedure for the specific compounds follows below.

### Synthesis of tert-butyl 2-amino-3-cyano-4,7-dihydrothieno[2,3-c]pyridine-6(5H)-carboxylate (JNK-3)

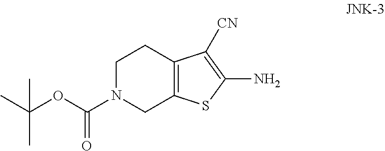 [SMILES: CC(C)(C)OC(=O)N1CCc2c(sc(N)c2C#N)C1]

1-Boc-4-piperidone (9 mmol), malononitrile (9 mmol) and sulphur (9 mmol) were dissolved in ethanol (50 ml). The mixture was heated to 50° C. and, once the solubilization of sulphur was complete, morpholine (9 mmol) was added dropwise. The reaction mixture was kept at 50° C. until the absence of starting material was confirmed by TLC and LCMS/MS (usually between 1 and 3 hours). Once cooled to room temperature, the desired aminothiophene precipitated, was filtered, and washed with ethanol until the mother liquor became colorless. ¹H NMR (500 MHz, CDCl₃) δ 4.71 (s, 2H), 4.29 (s, 2H), 3.59 (t, J=5.8 Hz, 2H), 2.52 (d, J=5.7 Hz, 2H), 1.41 (s, 9H). ¹³C NMR (126 MHz, CDCl₃) δ 160.86, 154-59, 114.85, 88.20, 80.42, 42.59, 42.04, 41.37, 28.42, 24.73.

### Synthesis of tert-butyl 2-(2-chloroacetamido)-3-cyano-4,5,6,7-tetrahydrobenzo[b]thiophene-6-carboxylate (PKL-B82)

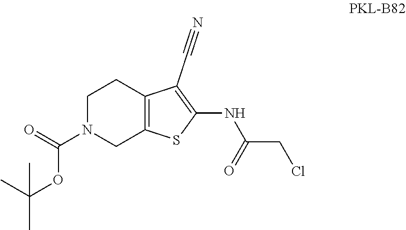 [SMILES: CC(C)(C)OC(=O)N1CCc2c(sc(NC(=O)CCl)c2C#N)C1]

To solution of Amine (200 mg, 0.71 mmol) in DMF, Triethylamine (0.11 mL, 0.78 mmol) was added dropwise to the reaction mixture at 0° C. The mixture was stirred for 30 min at room temperature. Then Chloro acetyl chloride (0.11 mL, 0.79 mmol) was added, and the reaction mixture was stirred at room temperature for 1h. The residue was poured to ice, and then extracted with Chloroform. The combined organic extracts were washed with brine, dried over magnesium sulphate and evaporated to dryness under reduced pressure to give the desired product as a yellow solid.

### Synthesis of tert-butyl 2-(2-(1H-indol-1-yl))acetamido)-3-cyano-4,7-dihydrothieno[2,3-c]pyridine-6(5H)-carboxylate (PKL-B73)

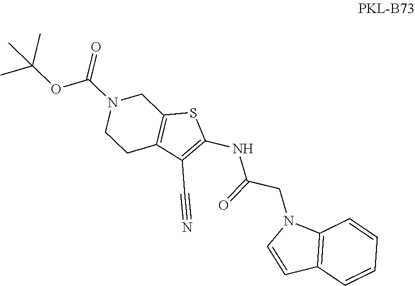 [SMILES: CC(C)(C)OC(=O)N1CCc2c(sc(NC(=O)Cn3ccc4ccccc43)c2C#N)C1]

Following general procedure D, PKL-B82 (80.0 mg, 0.22 mmol), was reacted with indole (26.41 mg, 0.22 mmol), to afford 82 mg (83%) of the title compound as a white solid after column chromatography (EtOAc in Hexane: 0-50%). ¹H NMR (500 MHz, CDCl₃) δ 8.41 (s, 1H), 7.63 (d, J=7.8 Hz, 1H), 7.24-7.17 (m, 3H), 7.12 (ddd, J=8.0, 6.7, 1.4 Hz, 1H), 7.09 (d, J=3.2 Hz, 1H), 6.64 (d, J=3.2 Hz, 1H), 4.94 (s, 2H), 4.39 (s, 2H), 3.57 (t, J=5.9 Hz, 2H), 2.52 (t, J=5.8 Hz, 2H), 1.39 (s, 10H). ¹³C NMR (126 MHz, CDCl₃) δ 165-72, 154.49, 146.35, 136.18, 129.02, 127.80, 123.06, 121.83, 120.90, 112.93, 108.87, 104.66, 94.34, 80.62, 49.37, 31.60, 28.40, 22.67, 14.14.

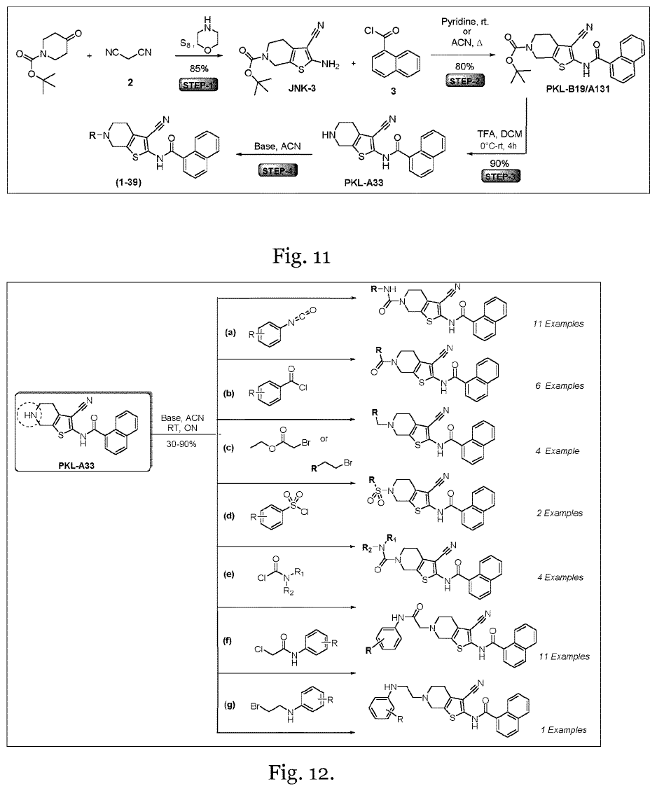

A schematic view of the synthesis of N-(3-cyano-4,5,6,7-tetrahydrothieno[2,3-c]pyridin-2-yl)-1-naphthamide can be seen in FIG. 11-17 and the synthesis procedure for the specific compounds follows below.

### Synthesis of N-(3-cyano-4,5,6,7-tetrahydrothieno[2,3-c]pyridin-2-yl)-1-naphthamide (PKL-B19/A131)

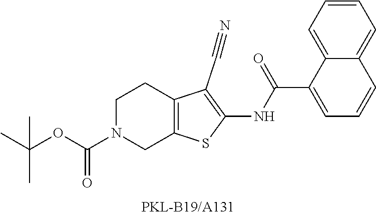 [SMILES: CC(C)(C)OC(=O)N1CCc2c(sc(NC(=O)c3cccc4ccccc34)c2C#N)C1]

This synthesis can be performed using acetonitrile (ACN) (i) or pyridine as solvent (ii) (step 2 in FIG. 11).
(i): A solution of JNK-3 (1 g, 3.58 mmol) in ACN (5 ml) with 1-naphthoyl chloride (1 mL, 3.94 mmol) was stirred together at 80° C. under an inert atmosphere for 1h. The reaction was monitored by TLC and LCMS/MS.
(ii): A solution of JNK-3 (1 g, 3.58 mmol) in pyridine (3 ml) with 1-naphthoyl chloride (1 mL, 3.94 mmol) was stirred together at room temperature under an inert atmosphere overnight. The reaction was monitored by TLC and LCMS/MS. The mixture was then evaporated to dryness and chromatographed over silica gel using with a gradient of hexane/EtOAc (9:1 to 7:3). The product fractions were collected and evaporated to afford 80% of the title compound.

¹H NMR (500 MHz, CDCl₃) δ 8.03 (dd, J=1.1, 8.3 Hz, 2H), 7.76 (dd, J=1.1, 7.1 Hz, 2H), 7.54-7.49 (m, 2H), 7.45 (ddd, J=1.4, 6.8, 8.5 Hz, 2H), 7.42-7.35 (m, 4H), 7.00-6.95 (m, 2H), 4.65 (s, 2H), 3.78 (s, 2H), 2.87 (d, J=5.7 Hz, 2H), 1.51 (s, 9H). ¹³C NMR (126 MHz, CDCl₃) δ 171.38, 146.00, 132.98, 132.21, 131.69, 129.46, 128.10, 127.60, 126.96, 126.68, 124.75, 123.97, 113.08, 80.80, 28.43, 24.74.

### Synthesis of N-(3-cyano-4,5,6,7-tetrahydrothieno[2,3-c]pyridin-2-yl)-1-naphthamide (PKL-A33)

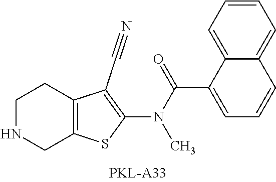 [SMILES: CN(C(=O)c1cccc2ccccc12)c1sc2c(c1C#N)CCNC2]

Following the general procedure G, PKL-B19/A131 (1 g, 2.31 mmol) in methylene chloride (DCM, 5.5 mL), reacted with TFA (1 mL) to afford 90% the desired product as a white solid after column chromatography using methanol in EtOAc: 95-5%.

General Procedure for the Synthesis of N-functionalization of N-(0.3-cyano-4,5,6,7-tetrahydrothieno[2,3-c]pyridin-2-yl)-1-naphthamide (1-24)

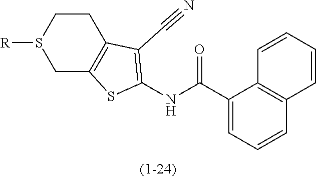

To a solution of the PKL-A33 in acetonitrile (0.1 M), triethylamine (1.2 equiv.) and the selected acylchloride/isocyanate/carbamoyl chlorides/alkyl halide/benzoyl chlorides/sulfonyl chloride (1 to 1.2 equiv.) was added. The reaction mixture was stirred at room temperature for overnight, concentrated, mounted on silica and purified by flash chromatography to afford the desired compounds.

### Synthesis of Representative Molecule PKL-A152

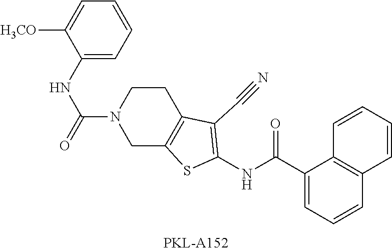 [SMILES: COc1ccccc1NC(=O)N1CCc2c(sc(NC(=O)c3cccc4ccccc34)c2C#N)C1]

Following step 1-3 (see FIG. 11), to obtain N-(3-cyano-4,5,6,7-tetrahydrothieno[2,3-c]pyridin-2-yl)-1-naphthamide.

First step, 1-Boc-4-piperidone (9 mmol), malononitrile (9 mmol) and sulphur (9 mmol) were dissolved in ethanol (50 ml). The mixture was heated to 50° C. and, once the solubilization of sulphur was complete, morpholine (9 mmol) was added dropwise. The reaction mixture was kept at 50° C. until the absence of starting material was confirmed by TLC and LCMS/MS (usually between 1 and 3 hours). Once cooled to room temperature, the desired aminothiophene precipitated, was filtered, and washed with ethanol until the mother liquor became colorless.

Second step can be performed using acetonitrile (ACN) (i) or pyridine as solvent (ii) as mentioned above.
(i) A solution of JNK-3 (1 g, 3.58 mmol) in ACN (5 ml) with 1-naphthoyl chloride (1 mL, 3.94 mmol) was stirred together at 80° C. under an inert atmosphere for 1 h. The reaction was monitored by TLC and LCMS/MS.
(ii) A solution of JNK-3 (1 g, 3.58 mmol) in pyridine (3 ml) with 1-Naphthoyl chloride (1 mL, 3.94 mmol) was stirred together at room temperature under an inert atmosphere overnight. The reaction was monitored by TLC and LCMS/MS.

The mixture was then evaporated to dryness and chromatographed over silica gel eluting using a gradient of hexane/EtOAc (9:1 to 7:3). The product fractions were collected and evaporated to afford the title compound.

Third step, to a solution of protected amine in methylene chloride (DCM), trifluoroacetic acid (TFA) (DCM:TFA 4:1) was added in the presence of nitrogen. The mixture was stirred at room temperature for 4 hours. The reaction progress and results were confirmed by TLC and LCMS/MS. When the reaction was completed, the reaction mixture was concentration under reduced pressure, and crystallized by adding ether or ethanol to obtain the target compound PKL-A33.

Step 4, to a solution of the PKL-A33 in acetonitrile (0.1 M), Triethylamine (1.2 equiv.) and the 1-isocyanato-2-methoxybenzene (1.2 equiv.) was added. The reaction mixture was stirred at room temperature for 2 hours, concentrated, mounted on silica and purified by flash chromatography to afford the desired compounds.

¹H NMR (500 MHz, DMSO) δ 12.43 (s, 1H), 8.18-8.12 (m, 2H), 8.09-8.03 (m, 1H), 7.91 (s, 1H), 7.84 (dd, J=1.2, 7.1 Hz, 1H), 7.69-7.62 (m, 3H), 7.58 (dd, J=1.6, 7.9 Hz, 1H), 7.09-7.00 (m, 2H), 6.89 (td, J=1.7, 7.5 Hz, 1H), 4.65 (d, J=1.8 Hz, 2H), 3.83 (s, 3H), 3.80 (t, J=5.7 Hz, 2H), 2.72 (t, J=5.7 Hz, 2H). ¹³C NMR (126 MHz, DMSO) δ 167.30, 155-51, 151.30, 147.67, 133.53, 132.19, 131.62, 131.02, 130.21, 128.97, 128.70, 127.89, 127.28, 127.01, 125.84, 125.39, 125.19, 124.50, 123.94, 120.63, 114.28, 111.53, 94.75, 56.14, 43.04, 41.62, 24.27.

### Synthesis of Representative Molecule PKL-A157

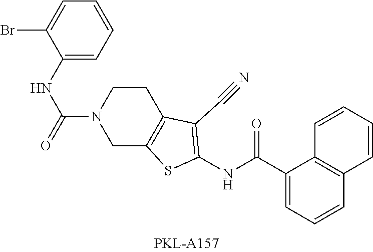 [SMILES: N#Cc1c(NC(=O)c2cccc3ccccc23)sc2c1CCN(C(=O)Nc1ccccc1Br)C2]

Following steps 1-3 (see FIG. 11), to obtain N-(3-cyano-4,5,6,7-tetrahydrothieno[2,3-c]pyridin-2-yl)-1-naphthamide.

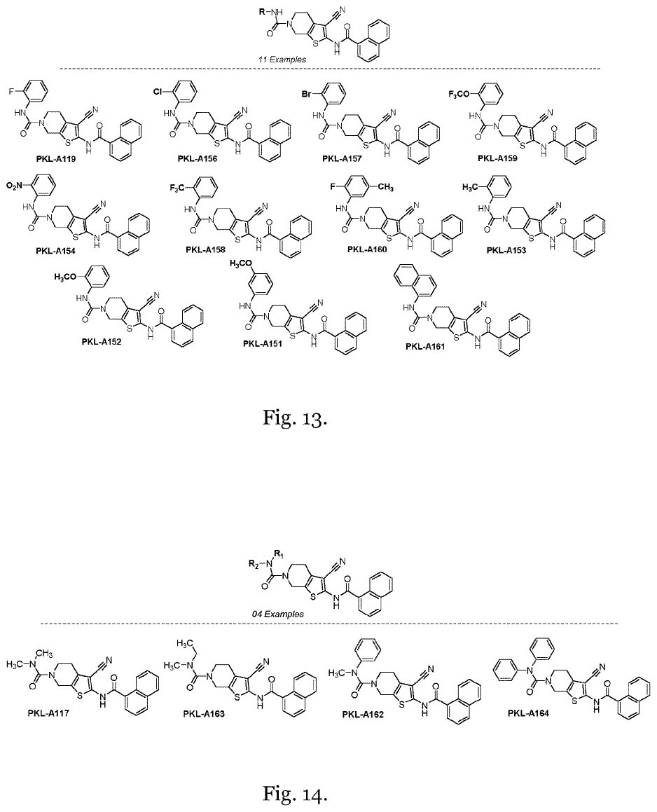

Step 4, to a solution of the PKL-A33 in acetonitrile (0.1 M), triethylamine (1.2 equiv.) and 1-bromo-2-isocyanatobenzene (1.2 equiv.) was added. The reaction mixture was stirred at room temperature for 2 hours, concentrated, mounted on silica and purified by flash chromatography to afford the desired compounds (see FIG. 13).

¹H NMR (500 MHz, DMSO) δ 12.43 (s, 1H), 8.45 (s, 1H), 8.18-8.11 (m, 2H), 8.09-8.04 (m, 1H), 7.84 (dd, J=1.2, 7.1 Hz, 1H), 7.68-7.61 (m, 4H), 7.48 (dd, J=1.6, 8.0 Hz, 1H), 7.36 (td, J=1.5, 7.7 Hz, 1H), 7.12 (td, J=1.7, 7.7 Hz, 1H), 4.67 (d, J=1.7 Hz, 2H), 3.83 (t, J=5.7 Hz, 2H), 2.74 (t, J=5.7 Hz, 2H). ¹³C NMR (126 MHz, DMSO) δ 167.31, 155.54, 147.66, 138.19, 133.53, 132.91, 132.18, 131.63, 131.01, 130.22, 128.98, 128.60, 128.37, 127.90, 127.29, 127.07, 127.02, 125.40, 125.20, 120.58, 114.28, 94.81, 43.05, 41.78, 24.30.

### Synthesis of Representative Molecule PKL-A158

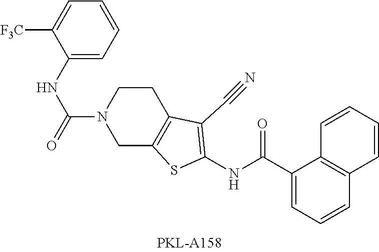 [SMILES: N#Cc1c(NC(=O)c2cccc3ccccc23)sc2c1CCN(C(=O)Nc1ccccc1C(F)(F)F)C2]

Following steps 1-3 (see FIG. 11), to obtain N-(3-cyano-4,5,6,7-tetrahydrothieno[2,3-c]pyridin-2-yl)-1-naphthamide.

Step 4, to a solution of the PKL-A33 in acetonitrile (1 equiv., 0.1 M), triethylamine (1.2 equiv.) and 1-isocyanato-2-(trifluoromethyl) benzene (1.2 equiv.) was added. The reaction mixture was stirred at room temperature for 2 hours, concentrated, mounted on silica and purified by flash chromatography to afford the desired compounds (see FIG. 13).

¹H NMR (500 MHz, DMSO) δ 12.43 (s, 1H), 8.51 (s, 1H), 8.15 (t, J=8.0 Hz, 2H), 8.08-8.04 (m, 1H), 7.84 (dd, J=1.2, 7.0 Hz, 1H), 7.71 (dd, J=1.5, 7.8 Hz, 1H), 7.64 (qdd, J=2.1, 7.0, 10.5 Hz, 4H), 7.44 (dd, J=7.8, 11.2 Hz, 2H), 4.64 (t, J=1.7 Hz, 2H), 3.81 (t, J=5.7 Hz, 2H), 2.71 (t, J=5.7 Hz, 2H). ¹³C NMR (126 MHz, DMSO) δ 166.36, 155.42, 132.56, 132.29, 130.65, 130.44, 129.97, 129.25, 128.00, 126.92, 126.32, 126.04, 125.80, 124.42, 124.24, 42.03, 40.80, 39.52, 23.18.

### Synthesis of Representative Molecule PKL-A164

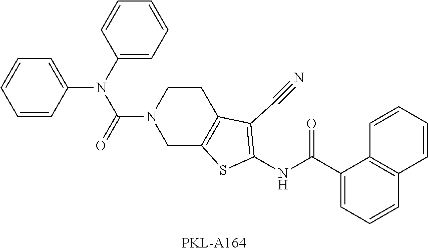 [SMILES: N#Cc1c(NC(=O)c2cccc3ccccc23)sc2c1CCN(C(=O)N(c1ccccc1)c1ccccc1)C2]

Following steps 1-3 (see FIG. 11), to obtain N-(3-cyano-4,5,6,7-tetrahydrothieno[2,3-c]pyridin-2-yl)-1-naphthamide.

Step 4, to a solution of the PKL-A33 in acetonitrile (1 eqiuv., 0.1 M), Triethylamine (1.2 equiv.) and diphenylcarbamic chloride (1.2 equiv.) was added. The reaction mixture was stirred at room temperature for 6 hours, concentrated, mounted on silica and purified by flash chromatography to afford the desired compounds (see FIG. 14).

¹H NMR (500 MHz, DMSO) δ 11.57 (s, 1H), 7.32 (dt, J=1.0, 8.3 Hz, 1H), 7.30-7.27 (m, 1H), 7.24-7.20 (m, 1H), 6.99 (dd, J=1.2, 7.2 Hz, 1H), 6.84-6.76 (m, 3H), 6.56-6.50 (m, 4H), 6.38-6.32 (m, 2H), 6.25-6.20 (m, 4H), 3.63 (d, J=1.8 Hz, 2H), 2.79 (t, J=5.6 Hz, 2H), 1.46 (t, J=5.7 Hz, 2H). ¹³C NMR (126 MHz, DMSO) δ 170.81, 167.28, 159.63, 145.05, 133.52, 132.17, 131.61, 130.62, 130-19, 129.80, 128.97, 127.89, 127.26, 127.01, 125.55, 125.38, 125.28, 125-17, 114.18, 60.23, 44-13, 43-41.

### Synthesis of Representative Molecule PKL-A162

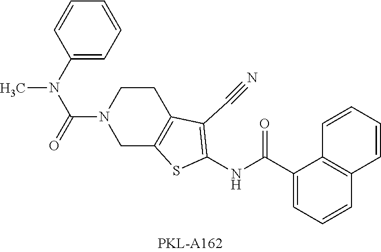 [SMILES: CN(C(=O)N1CCc2c(sc(NC(=O)c3cccc4ccccc34)c2C#N)C1)c1ccccc1]

Following steps 1-3 (see FIG. 11), to obtain N-(3-cyano-4,5,6,7-tetrahydrothieno[2,3-c]pyridin-2-yl)-1-naphthamide.

Step 4, to a solution of the PKL-A33 in acetonitrile (1 equiv., 0.1 M), triethylamine (1.2 equiv.) and methyl(phenyl)carbamic chloride (1.2 equiv.) was added. The reaction mixture was stirred at room temperature for 6 hours, concentrated, mounted on silica and purified by flash chromatography to afford the desired compounds (see FIG. 14).

¹H NMR (500 MHz, DMSO) δ 11.55 (s, 1H), 7.31 (d, J=8.2 Hz, 1H), 7.27 (d, J=7.3 Hz, 1H), 7.24-7.18 (m, 2H), 6.98 (d, J=7.2 Hz, 1H), 6.82-6.76 (m, 4H), 6.56 (t, J=7.9 Hz, 2H), 6.38 (d, J=7.7 Hz, 2H), 6.35 (d, J=7.3 Hz, 1H), 3.45 (s, 2H), 2.60 (d, J=5.8 Hz, 2H), 2.31 (d, J=3.5 Hz, 3H). ¹³C NMR (126 MHz, DMSO) δ 167.29, 160.80, 146.67, 133.51, 131.59, 130.67, 130-19, 129.97, 128.96, 127.87, 127.25, 126.99, 125.37, 125.19, 125.04, 124.14, 44.38, 43.36, 23.75, 14.56.

### Synthesis of Representative Molecule PKL-A16.3

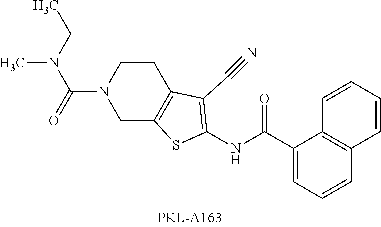 [SMILES: CCN(C)C(=O)N1CCc2c(sc(NC(=O)c3cccc4ccccc34)c2C#N)C1]

Following steps 1-3 (see FIG. 11), to obtain N-(3-cyano-4,5,6,7-tetrahydrothieno[2,3-c]pyridin-2-yl)-1-naphthamide.

Step 4, to a solution of the PKL-A33 in acetonitrile (1 equiv., 0.1 M), triethylamine (1.2 equiv.) and the methyl(ethyl)carbamic chloride (1.2 equiv.) was added. The reaction mixture was stirred at room temperature for 6 hours, concentrated, mounted on silica and purified by flash chromatography to afford the desired compounds.

¹H NMR (500 MHz, DMSO) δ 11.55 (s, 1H), 7.32 (dd, J=1.2, 8.3 Hz, 1H), 7.31-7.27 (m, 1H), 7.24-7.21 (m, 1H), 6.99 (dd, J=1.2, 7.1 Hz, 1H), 6.84-6.77 (m, 3H), 3.47 (d, J=1.8 Hz, 2H), 2.61 (t, J=5.6 Hz, 2H), 2.34 (q, J=7.1 Hz, 2H), 1.97 (s, 3H), 1.88-1.84 (m, 2H), 0.26 (t, J=7.1 Hz, 3H). ¹³C NMR (126 MHz, DMSO) δ 167.27, 163-74, 133.51, 132.23, 131.58, 130.90, 130.20, 128.96, 127.87, 127.23, 127.00, 125.38, 125.18, 45.42, 44.65, 44.26, 35.54, 24.12, 12.88.

### Synthesis of Representative Molecule PKL-A170

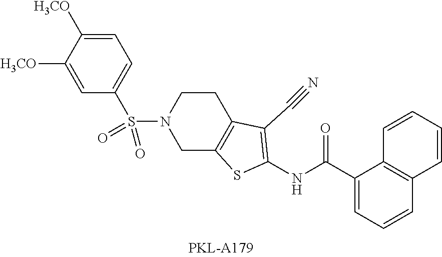 [SMILES: COc1ccc(S(=O)(=O)N2CCc3c(sc(NC(=O)c4cccc5ccccc45)c3C#N)C2)cc1OC]

Following steps 1-3 (see FIG. 11), to obtain N-(3-cyano-4,5,6,7-tetrahydrothieno[2,3-c]pyridin-2-yl)-1-naphthamide PKL-A33.

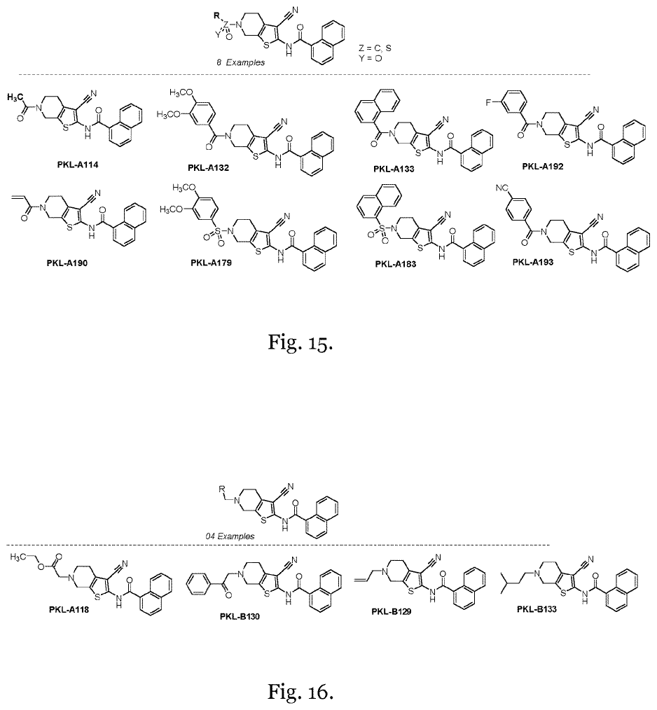

Step 4, to a solution of PKL-A33 in acetonitrile (1 equiv., 0.1 M), triethylamine (1.2 equiv.) and 3,4-dimethyl sulfonyl chloride (1.2 equiv.) were added. The reaction mixture was stirred at room temperature for 3 hours, concentrated, mounted on silica and purified by flash chromatography to afford the desired compounds (see FIG. 15).

¹H NMR (500 MHz, DMSO) δ 11.59 (s, 1H), 7.31 (dd, J=1.1, 8.3 Hz, 1H), 7.27 (dd, J=1.5, 8.2 Hz, 1H), 7.24-7.21 (m, 1H), 6.98 (dd, J=1.2, 7.1 Hz, 1H), 6.83-6.77 (m, 4H), 6.60 (dd, J=2.2, 8.5 Hz, 1H), 6.38 (d, J=2.2 Hz, 1H), 6.33 (d, J=8.5 Hz, 1H), 3.50 (d, J=1.8 Hz, 2H), 3.01 (d, J=7.2 Hz, 6H), 2.61 (t, J=5.9 Hz, 2H), 1.77 (t, J=5.9 Hz, 2H). ¹³C NMR (126 MHz, DMSO) δ 167.31, 153.09, 149.21, 147.95, 133.51, 132.12, 131.62, 130.18, 130.11, 128.97, 128.59, 127.90, 127.28, 127.02, 125.38, 125.14, 123.67, 121.64, 114.06, 111.88, 110.25, 94.15, 56.40, 56.32, 44.67, 43.29, 23.79.

### Synthesis of Representative Molecule PKL-A114

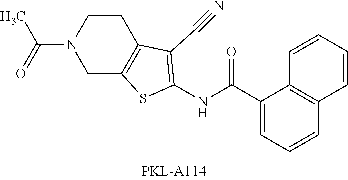 [SMILES: CC(=O)N1CCc2c(sc(NC(=O)c3cccc4ccccc34)c2C#N)C1]

Following steps 1-3 (see FIG. 11), to obtain N-(3-cyano-4,5,6,7-tetrahydrothieno[2,3-c]pyridin-2-yl)-1-naphthamide.

Step 4, to a solution of the PKL-A33 in acetonitrile (0.1 M), triethylamine (1.2 equiv.) and acetic anhydride (1.2 equiv.) were added. The reaction mixture was stirred at room temperature for 4 hours, concentrated, mounted on silica and purified by flash chromatography to afford the desired compounds (see FIG. 15).

¹H NMR (500 MHz, DMSO) δ 11.60 (d, J=13.1 Hz, 1H), 7.35-7.29 (m, 2H), 7.25-7.22 (m, 1H), 7.01 (dd, J=1.2, 7.1 Hz, 1H), 6.82 (dddd, J=1.5, 5.1, 6.6, 9.6 Hz, 3H), 3.88-3.77 (m, 2H), 2.93 (dt, J=5.8, 13.6 Hz, 2H), 1.95-1.75 (m, 2H), 1.29 (d, J=17.6 Hz, 3H). ¹³C NMR (126 MHz, DMSO) δ 169.31, 147.71, 133.52, 132.15, 131.63, 131.12, 130.81, 130.20, 128.97, 127.89, 127.29, 127.01, 125.53, 125.38, 125.18, 114.27, 55.39, 44.56, 43.28, 24.89, 21.76.

### Synthesis of Representative Molecule PKL-A118

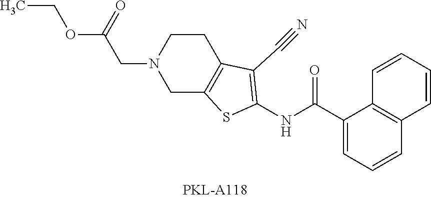 [SMILES: CCOC(=O)CN1CCc2c(sc(NC(=O)c3cccc4ccccc34)c2C#N)C1]

Following steps 1-3 (see FIG. 11), to obtain N-(3-cyano-4,5,6,7-tetrahydrothieno[2,3-c]pyridin-2-yl)-1-naphthamide PKL-A33.

Step 4, to a solution of the PKL-A33 in acetonitrile (1 equiv., 0.1 M), triethylamine (1.2 equiv.) and ethyl bromo acetate (2.0 equiv.) were added. The reaction mixture was stirred at room temperature for 4 hours, concentrated, mounted on silica and purified by flash chromatography to afford the desired compounds (see FIG. 16).

¹H NMR (500 MHz, DMSO) δ 8.89 (s, 1H), 8.41 (dd, J=1.2, 8.4 Hz, 1H), 8.05 (dt, J=1.1, 8.3 Hz, 1H), 7.96-7.91 (m, 1H), 7.82 (dd, J=1.2, 7.0 Hz, 1H), 7.61-7.52 (m, 3H), 4.23 (q, J=7.1 Hz, 2H), 3.83 (t, J=1.8 Hz, 2H), 3.45 (s, 2H), 2.99 (t, J=5.8 Hz, 2H), 2.78 (t, J=5.7 Hz, 2H), 1.30 (s, 3H). ¹³C NMR (126 MHz, DMSO) δ 165.47, 160.70, 142.53, 129.10, 127.95, 125.98, 125.47, 124.68, 123.92, 123.29, 122.26, 121.43, 120.21, 119.89, 109.22, 88.85, 56.17, 53.17, 45.52, 44.65, 24.96, 19.32, 9.52, −3.72.

### Preparation of intermediate 1-(2-bromoethyl)-1H-benzo[d]imidazole

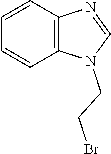 [SMILES: BrCCn1cnc2ccccc21]

A solution of benzimidazol (200 mg, 1.69 mmol), potassium hydroxide (104.48 mg, 1.86 mmol) and tetrabutylammonium bromide (TBAB, catalytic amount 0.02 equiv.) in THF was stirred for 30 min at room temperature. Then dibromoethane (0.5 mL, 1.69 mmol) was added, and the reaction mixture was stirred at room temperature for 18 h. The residue was partitioned between water and ethyl acetate and the aqueous extracted with further ethyl acetate. The combined organic extracts were washed with brine, dried over magnesium sulphate and evaporated to dryness under reduced pressure to afford 250 mg (65%) the desired product as a white solid.

### N-(6-(2-(1H-benzo[d]imidazol-1-yl)ethyl)-3-cyano-4,5,6,7-tetrahydrothieno[2,3c]pyridin-2-yl)-1-naphthamide (PKL-B153)

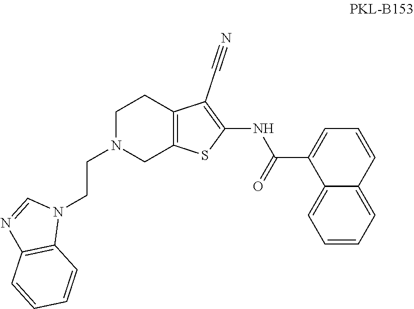 [SMILES: N#Cc1c(NC(=O)c2cccc3ccccc23)sc2c1CCN(CCn1cnc3ccccc31)C2]

Following steps 1-3 (see FIG. 11), to obtain N-(3-cyano-4,5,6,7-tetrahydrothieno[2,3-c]pyridin-2-yl)-1-naphthamide.

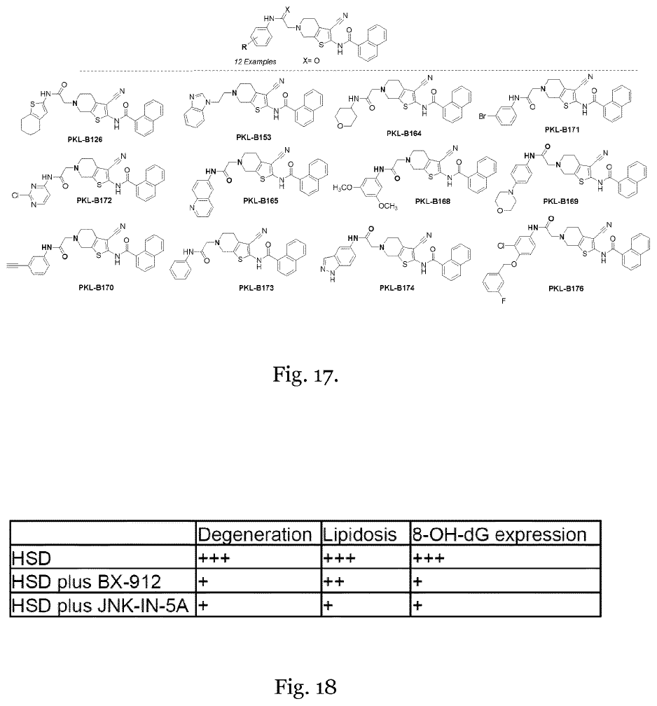

Step 4, following general procedure D, PKL-A33 ((100 mg, 2.99 mmol), was reacted with 1-(2-bromoethyl)-1H-benzo[d]imidazole (67.51 mL, 2.99 mmol), to afford 95 mg (66%) of the title compound as a yellow solid after column chromatography (MeOH in DCM: 0-10%) (see FIG. 17). ¹H NMR (500 MHz, DMSO) δ 8.31 (s, 1H), 8.13 (d, J=8.4 Hz, 1H), 8.08-8.04 (m, 1H), 7.92 (d, J=7.0 Hz, 1H), 7.75-7.58 (m, 6H), 7.29 (dt, J=32.7, 7.4 Hz, 2H), 4.51 (t, J=6.3 Hz, 2H), 3.70 (s, 2H), 2.99 (t, J=6.2 Hz, 2H), 2.89 (t, J=5.7 Hz, 2H), 2.63 (d, J=5.6 Hz, 2H). ¹³C NMR (126 MHz, DMSO) δ 144.87, 143.78, 134.40, 133.61, 131.92, 130.90, 130.55, 129.76, 128.78, 127.36, 127.22, 126.66, 125.87, 125.38, 122.64, 121.81, 121.73, 119.83, 111.01, 110.87, 55.90, 51.06, 49.55, 42.37, 24.11.

### Preparation of Intermediate 2-chloro-N-(3-cyano-4,5,6,7-tetrahydrobenzo[b]thiophen-2-yl) acetamide (PKL-B83)

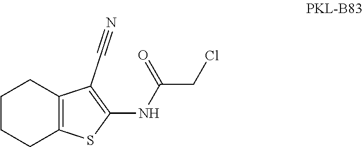 [SMILES: N#Cc1c(NC(=O)CCl)sc2c1CCCC2]

To solution of aminothiophene (200 mg, 1.12 mmol) in DMF, triethylamine (0.17 mL, 1.23 mmol) was added dropwise to the reaction mixture at 0° C. The mixture was stirred for 30 min at room temperature. Then chloroacetyl chloride (0.1 mL, 1.23 mmol) was added, and the reaction mixture was stirred at room temperature for 1h. The residue was poured to ice, and then extracted with chloroform. The combined organic extracts were washed with brine, dried over magnesium sulphate and evaporated to dryness under reduced pressure to afford 230 mg (81%) of the desired product as a brown solid.

¹H NMR (500 MHz, CDCl₃) δ 9.22 (s, 1H), 4.21 (s, 2H), 2.59 (ddd, J=7.8, 5.3, 1.8 Hz, 2H), 2.54 (td, J=6.0, 1.8 Hz, 2H), 1.81-1.73 (m, 5H). ¹³C NMR (126 MHz, CDCl₃) δ 162.80, 145.03, 131.40, 129.46, 113.79, 95.15, 42.03, 24.03, 23.98, 23.03, 22.04.

### N-(3-cyano-6-(2-oxo-2-((4,5,6,7-tetrahydrobenzo[b]thiophen-2-yl)amino)ethyl)-4,5,6,7-tetrahydrothieno[2,3-c]pyridin-2-yl)-1-naphthamide (PKL-B126)

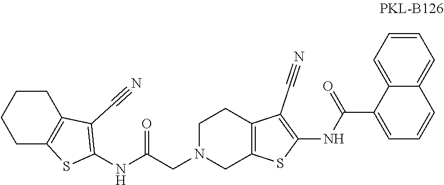 [SMILES: N#Cc1c(NC(=O)CN2CCc3c(sc(NC(=O)c4cccc5ccccc45)c3C#N)C2)sc2c1CCCC2]

Following steps 1-3 (see FIG. 11), to obtain N-(3-cyano-4,5,6,7-tetrahydrothieno[2,3-c]pyridin-2-yl)-1-naphthamide.

Step 4, following general procedure D, PKL-A33 ((100 mg, 2.99 mmol), was reacted with 1-(2-bromoethyl)-1H-benzo[d]imidazole (67.51 mL, 2.99 mmol), to afford 95 mg (66%) of the title compound as a yellow solid after column chromatography (EtOAc in Hexane: 0-50%). ¹H NMR (500 MHz, DMSO-d₆) δ 12.47 (s, 1H), 8.22 (dd, J=10.7, 7.9 Hz, 2H), 8.18-8.12 (m, 2H), 7.91 (d, J=6.9 Hz, 1H), 7.75-7.68 (m, 3H), 3.93 (h, J=6.4, 4.6 Hz, 2H), 3.71 (s, 2H), 3.07 (s, 2H), 2.79 (d, J=5.7 Hz, 2H), 2.72-2.65 (m, 3H), 1.84 (dt, J=7.7, 4.3 Hz, 5H). ¹³C NMR (126 MHz, DMSO) δ 167.25, 146.53, 133.53, 132.23, 131.60, 131.25, 131.22, 130.27, 130.22, 129.95, 128.97, 128.11, 127.87, 127.26, 127.00, 125.44, 125.38, 125.20, 114.63, 114.37, 94.69, 93.94, 50.46, 49.42, 29.50, 29.47, 23.98, 23.86, 23.07, 22.16.

### Results

The Treatment of Rats with Therapeutic Agents

To evaluate the potential toxicity of BX-912 and JNK-IN-5A in animals, three groups of 12-week-old rats (n=5) were raised. The first group was fed with CHOW (Ctrl) for seven days. The second group was fed with CHOW plus BX-912 (30 mg/kg) for seven days. The third group was fed with CHOW plus JNK-IN-5A (30 mg/kg) for seven days. After the seven days, all rats were sacrificed, and tissue samples from major organs and blood samples were collected for biosafety evaluation. The frequencies of micronucleated polychromatic erythrocytes (MNPCEs) in the blood samples were analysed, and it was found that the frequencies of MNPCEs in the drug-treated groups were not statistically different (p>0.05) when compared to Ctrl animals. The analysis indicated that both tested agents exhibited non-genotoxic potential. In addition, histopathological examinations were performed using the tissue samples obtained from the rats and it was found that kidney, small intestine, large intestine, stomach, heart, pancreas, muscle tissues and liver from all three rat groups exhibited normal histological structures. Moreover, immunohistochemical (IHC) examination on the liver tissues from the rats was performed and negative 8-OH-dG expression was observed in all three groups. Therefore, it was concluded that both BX-912 and JNK-IN-5A had no side effects on the rats.

Next, the efficacy of these two drugs in a steatosis rat study was investigated by feeding the animals with HSD. Five groups of 12-week-old rats (n=5) were raised. For two weeks, the first group was fed with CHOW and the remaining four groups were fed with HSD. At the end of week 2, the CHOW-fed rats (n=5) and one group of HSD-fed rats (n=5) were sacrificed for histopathological examination and it was confirmed that the HSD fed rats had already developed hepatic steatosis. One of the remaining groups was fed with HSD (HSD 3w) for seven days. Another one of the remaining groups was fed with HSD plus BX-912 (30 mg/kg) for seven days. The last one of the remaining groups was fed with HSD plus JNK-IN-5A (30 mg/kg) for seven days. After said seven days, all three groups were sacrificed, and tissue samples from the liver and other major organs and blood samples were collected for efficacy examination. A histopathological and immunohistochemical analysis was performed on the liver samples and other major organs obtained from the three rat groups. A standard histological structure on the small intestine, large intestine, stomach, heart and muscle tissues was observed in the examination of the drug-treated groups, and no toxic effect of the drugs on these tissues was found. In the rats from HSD 3w group, severe degeneration was detected in the renal tubule epithelium and severe fattening was detected in the parenchyma cells in the pancreatic tissues. In contrast, only mild degeneration in renal tubule epithelium and mild fattening in the parenchyma cells of pancreas tissues were detected in the drug-treated groups. In addition, severe degeneration and steatosis in hepatocytes in liver tissues of the rats from the HSD 3w group were observed. Mild degeneration and moderate steatosis were observed in hepatocytes in liver tissues of the HSD-fed rats treated with BX-12, whereas both degeneration and steatosis were mild in hepatocytes in liver tissues of the JNK-IN-5A-treated group (FIG. 3). Moreover, strong 8-OH-dG expression was observed in hepatocytes in liver tissues of the rats from HSD 3w group. In the drug-treated groups, the 8-OH-dG expression was however significantly decreased, see FIG. 18.

The rat studies, hence, showed that treating HSD fed rats with JNK-IN-5A decreased the degeneration and steatosis in hepatocytes in liver tissues.

The present inventors realized that the effect of the JNK-IN-5A can be tuned by modifying the chemical structure of the JNK-IN-5A molecule. Molecular docking was performed to select the target molecules which were then synthesised, and their effectiveness analysed.

### Analysis of Target Compounds

### In-Silico Analysis of Target Compounds

The In-silico studies revealed that due to the tetrahedral geometry of the sulfonyl group in the compounds A179 and A183, benzene and naphthalene ring are oriented away and as a result the main scaffold of the compound showed positional displacement and showed weaker interactions with the active site residues. These compounds are majorly involved in hydrophobic interaction with Lys93, Ile124 and Leu206. While the oxygen of the sulfonyl group of both compounds is involved in hydrogen bond interaction with Ala151 (3.3 and 3.4 Å) and Gln155 (3.2 and 3.4 Å), respectively.

The eleven urea derivatives (A159, A158, A152, A151, A119, A157, A161, A153, A156, A154 and A160) were synthesized through the reaction of the key intermediate (A33) with the appropriate isocyanates. Coupling A33 with several commercially available carbamoyl chlorides afforded different urea analogues. All further modifications of this position led to a significant increase in inhibition.

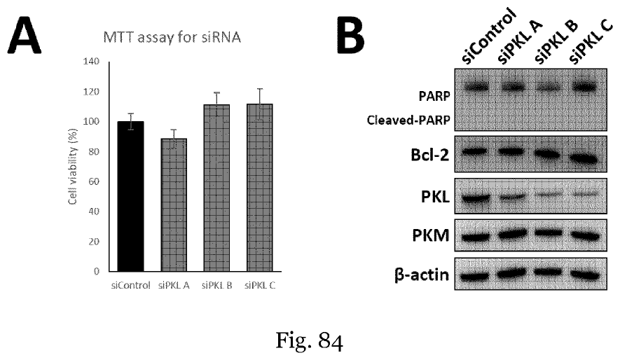

The piperidine ring with urea linker of the compounds A117, A119, A164, A156 A151, A152A153 and A154 have access to the solvent-exposed region and this bulky substitution allows the compound to remain in a similar conformation as the reference compound, JNK-IN-5A. Detail interaction analysis revealed that the benzamide moiety resides in the hydrophobic pocket which lies on the back side of the active site, and has mediated hydrophobic and pi-CH₃ interaction with the side chain of Lys93, Ile124 and Leu206. All these compounds stabilized the conserved hydrogen bond between the backbone amide of Met149 and the 3-cyano substituent of the main scaffold (2.3-3.3 Å). Another hydrogen bond is observed between the gatekeeper residue Met146 and the nitrile group with a distance range of 2.9-3.5 Å. furthermore, it was determined that steric interactions involving electron donating groups are significant for the activity (FIG. 84).

Hybrid derivatives A118, B130, B133 and B129 were prepared using corresponding alkylating agents in the presence of potassium carbonate. The case of compound B130, multiple hydrophilic interactions with the crucial active site residues Met146, Met149 and Asn152 at a distance of 3.5, 2.9 and 3.3 Å, were observed. These protein-ligand interactions were further stabilized by hydrophobic interaction with Lys93, Ile124 and Leu206. The acetophenone group linked to the main scaffold with the addition of methylene could stabilize the position of the ligand by interacting with Asn152. These results suggested that steric interactions are significant when the substituent size becomes relatively large as compared to the reference compound.

### In-Vitro Analysis of Target Compounds

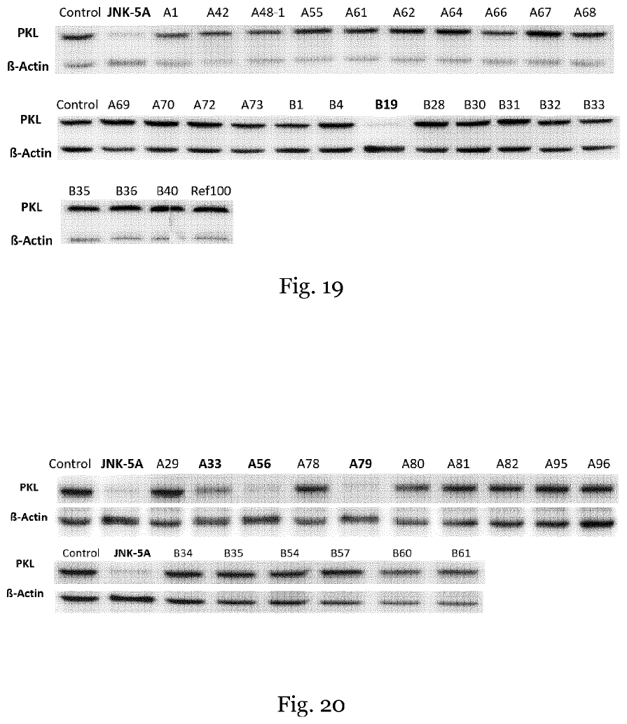

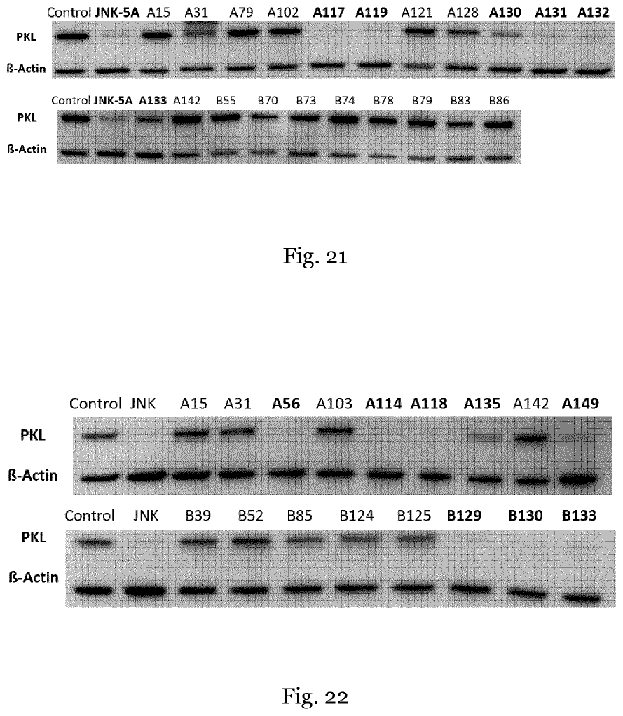

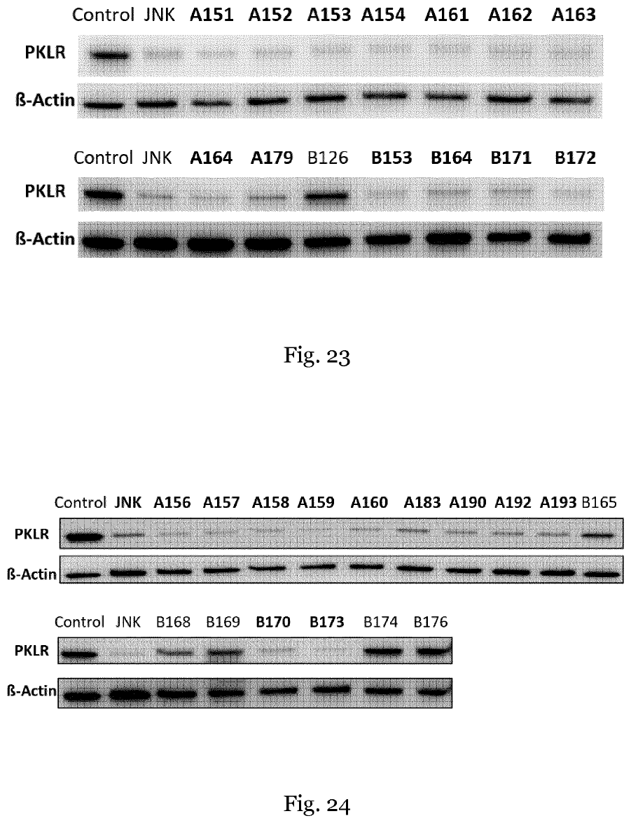

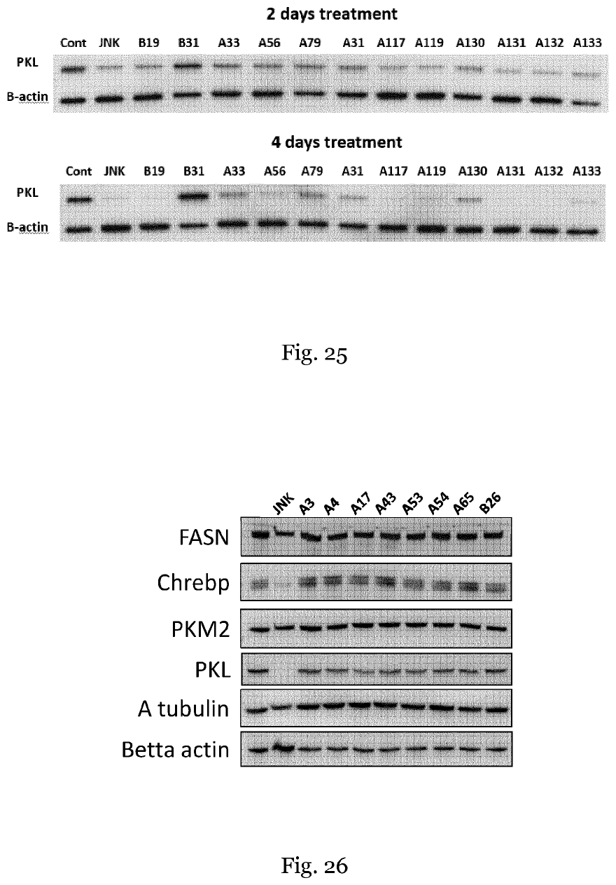

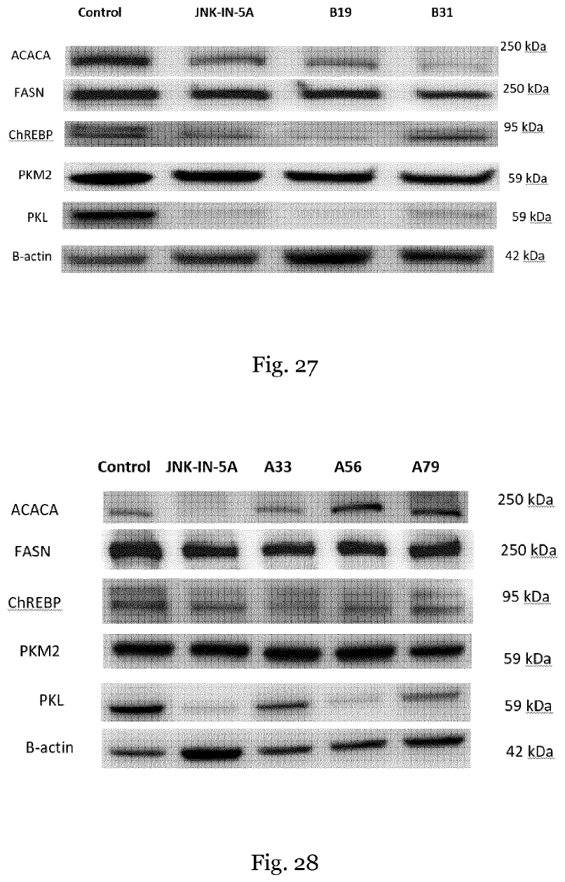

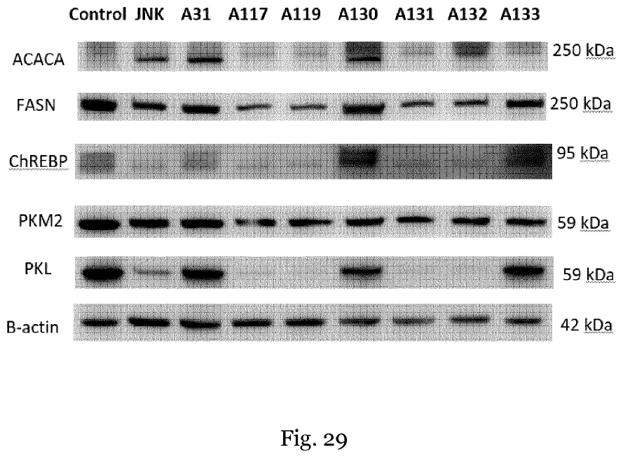

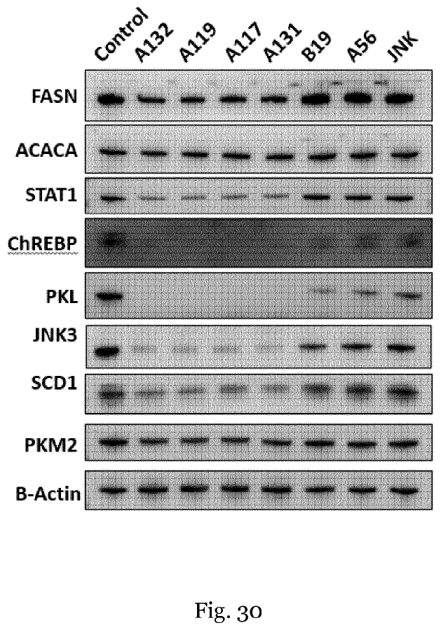

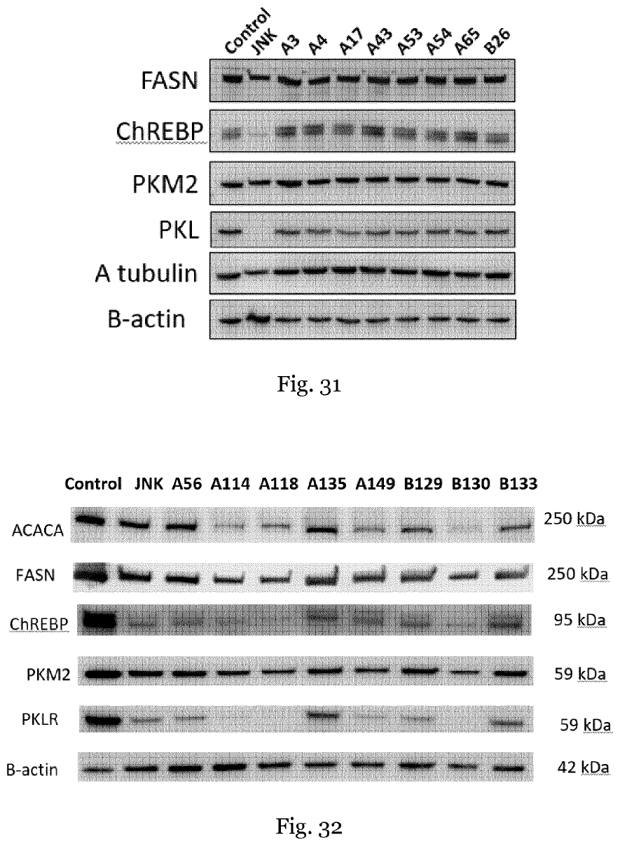

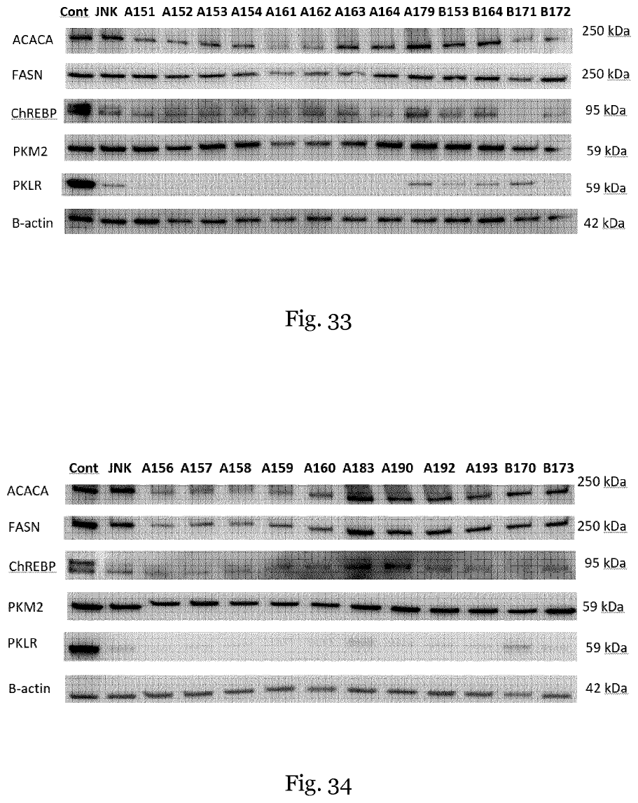

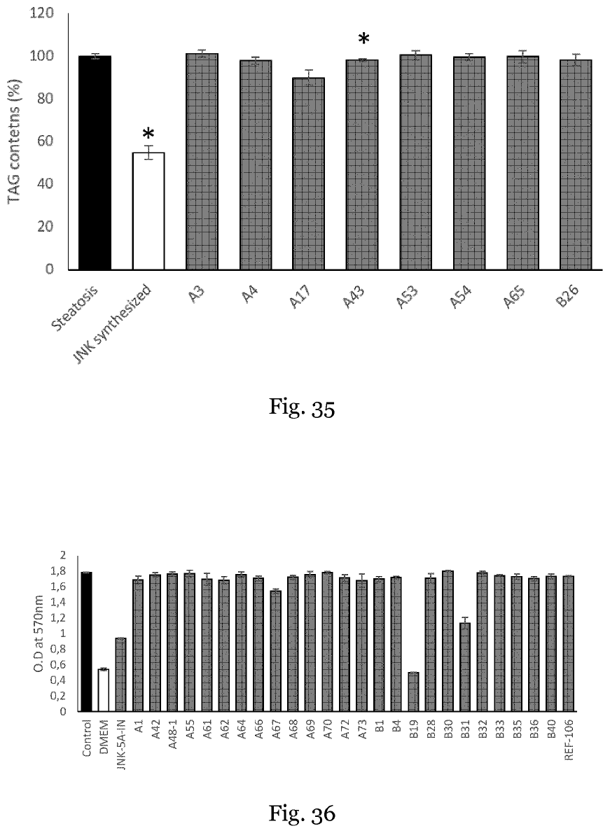

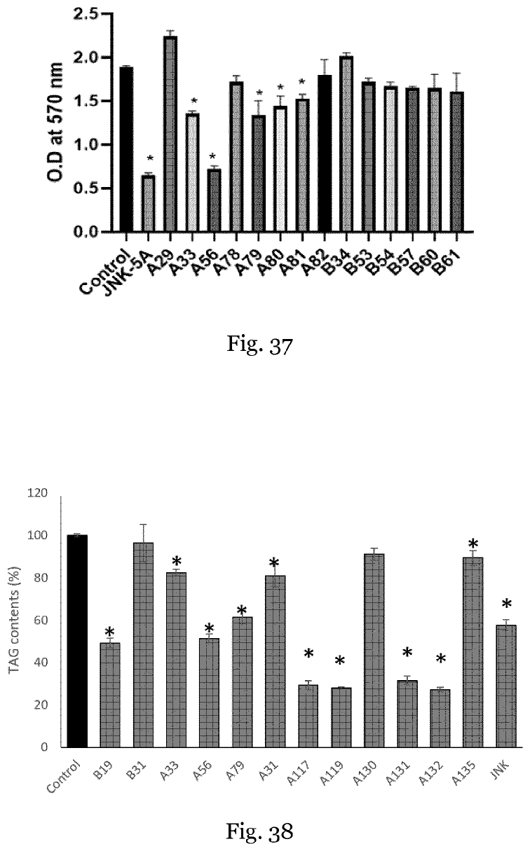

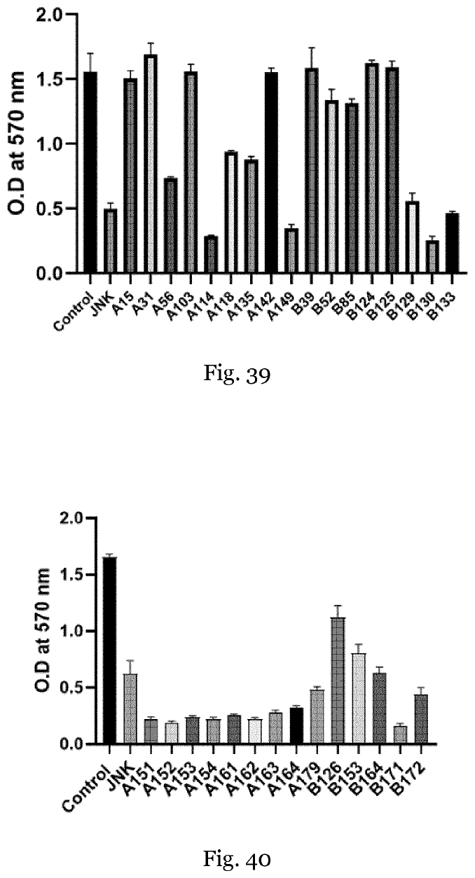

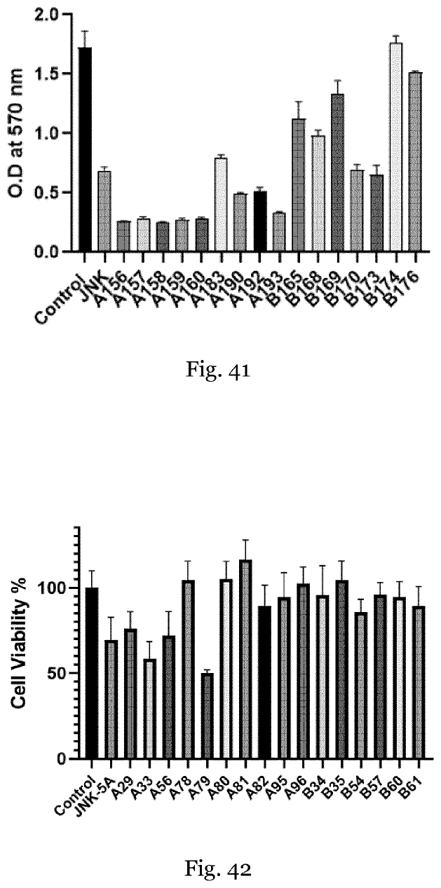

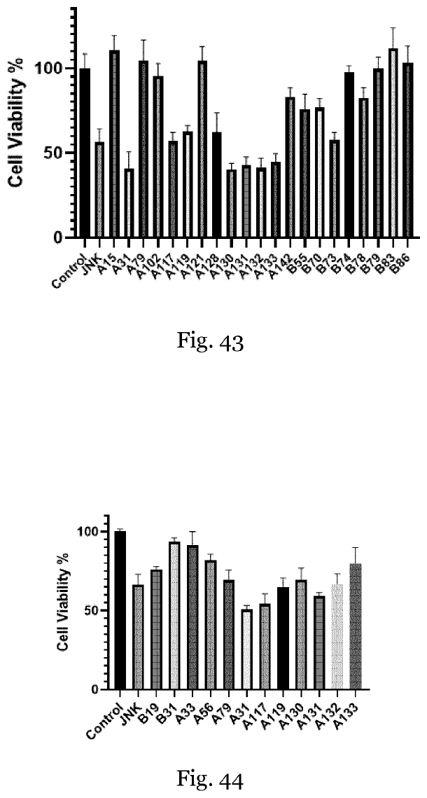

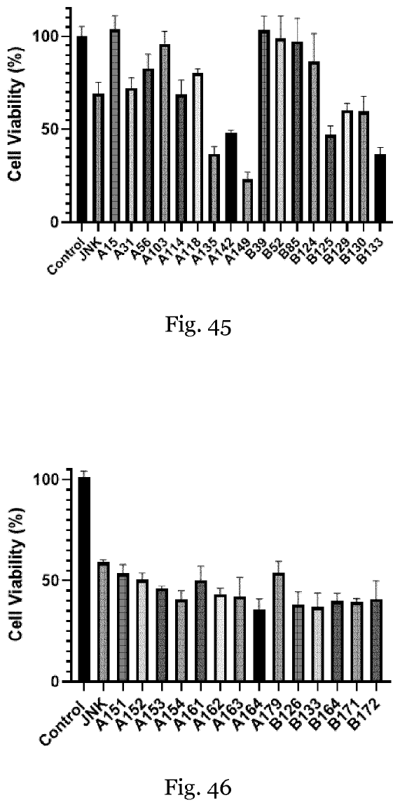

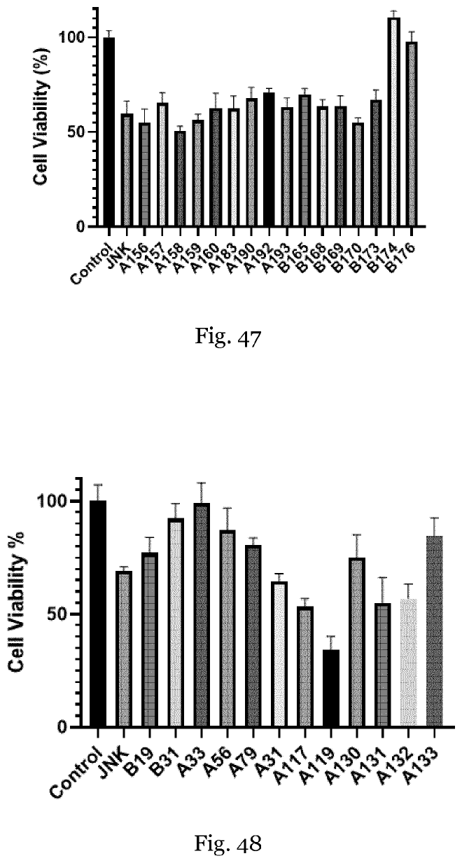

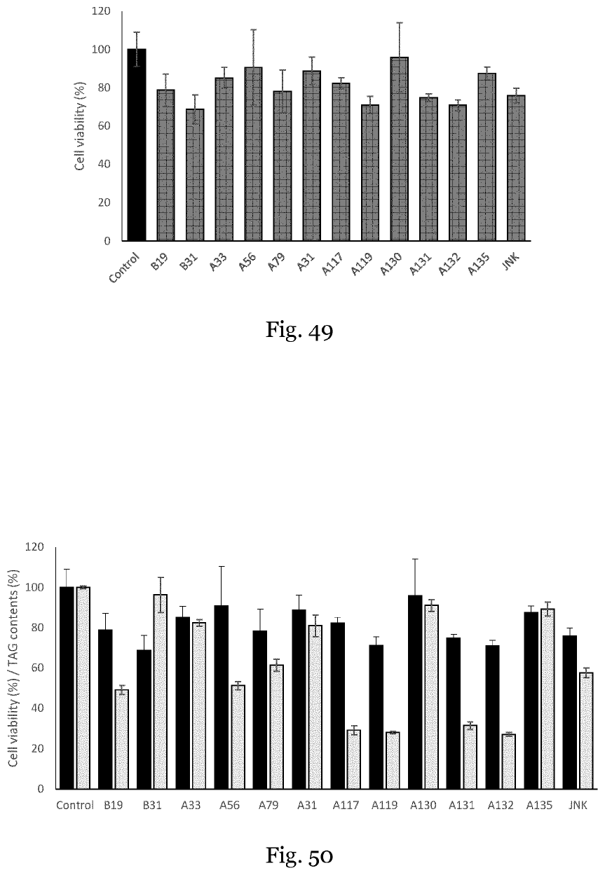

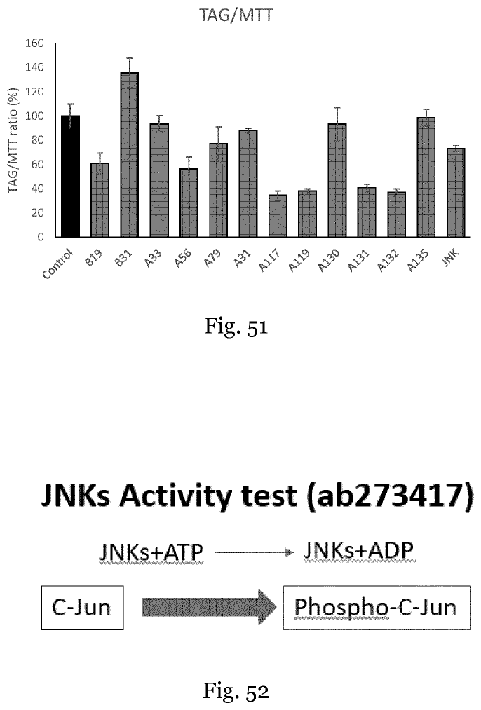

The activity of the synthesized target compounds was determined, see Table 1. All the active compounds resulted in reduced expression levels of PKLR (see FIG. 19-25) and other steatosis related proteins (see FIG. 26-34) as well as reduced TAG accumulation (see FIGS. 35-41 and 47-51). Most of the active compounds also resulted in reduced cell viability of HepG2 (see FIG. 42-50).

Treatment with the active compounds reduced PKL expression, while the treatment had no adverse effects on PKM expression (see FIG. 26-34). Thus, all active compounds might be considered PKL-specific compound with minimum side effects on other tissues.

TABLE 1
A list of all synthesized compounds and their effectiveness.

| Compound | Structure | Active | Not active |
|---|---|---|---|
| PKL-A17 | 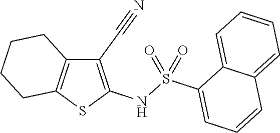 [SMILES: N#Cc1c(NS(=O)(=O)c2cccc3ccccc23)sc2c1CCCC2] | X | |
| PKL-A43 | 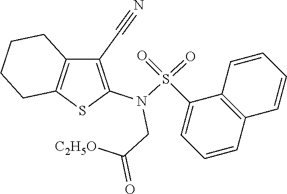 | | X |
| PKL-A44 | 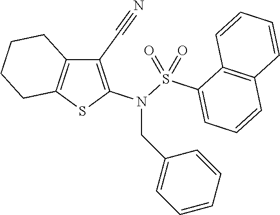 [SMILES: N#Cc1c(N(Cc2ccccc2)S(=O)(=O)c2cccc3ccccc23)sc2c1CCCC2] | | X |
| PKL-A53 | 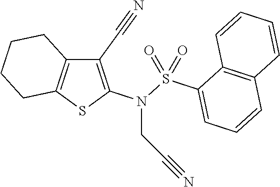 [SMILES: N#CCN(c1sc2c(c1C#N)CCCC2)S(=O)(=O)c1cccc2ccccc12] | | X |
| PKL-A54 | 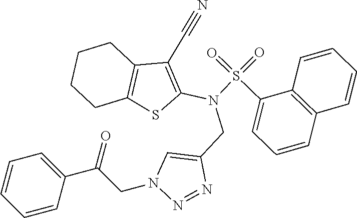 [SMILES: N#Cc1c(N(Cc2cn(CC(=O)c3ccccc3)nn2)S(=O)(=O)c2cccc3ccccc23)sc2c1CCCC2] | | X |
| PKL-A58 | 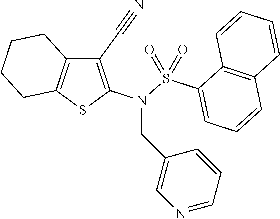 [SMILES: N#Cc1c(N(Cc2cccnc2)S(=O)(=O)c2cccc3ccccc23)sc2c1CCCC2] | | X |
| PKL-A2 | 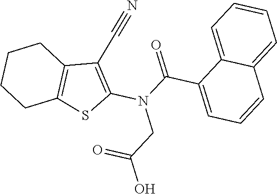 [SMILES: N#Cc1c(N(CC(=O)O)C(=O)c2cccc3ccccc23)sc2c1CCCC2] | | X |
| PKL-A3 | 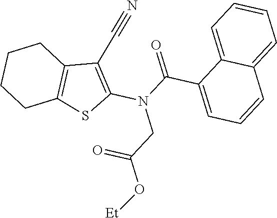 [SMILES: CCOC(=O)CN(C(=O)c1cccc2ccccc12)c1sc2c(c1C#N)CCCC2] | | X |
| PKL-A4 | 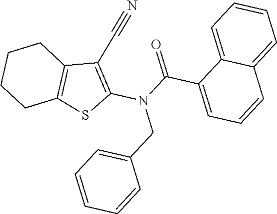 [SMILES: N#Cc1c(N(Cc2ccccc2)C(=O)c2cccc3ccccc23)sc2c1CCCC2] | | X |
| PKL-B29 | 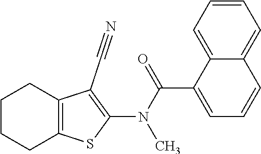 [SMILES: CN(C(=O)c1cccc2ccccc12)c1sc2c(c1C#N)CCCC2] | | X |
| PKL-A65 | 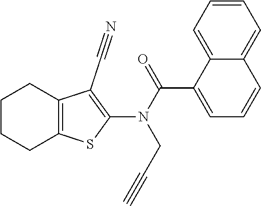 [SMILES: C#CCN(C(=O)c1cccc2ccccc12)c1sc2c(c1C#N)CCCC2] | | X |
| PKL-B26 | 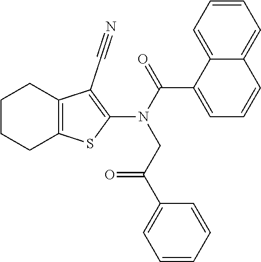 [SMILES: N#Cc1c(N(CC(=O)c2ccccc2)C(=O)c2cccc3ccccc23)sc2c1CCCC2] | | X |
| PKL-A66 | 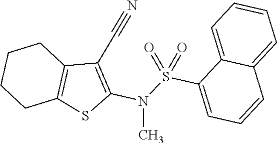 [SMILES: CN(c1sc2c(c1C#N)CCCC2)S(=O)(=O)c1cccc2ccccc12] | | X |
| PKL-A42 | 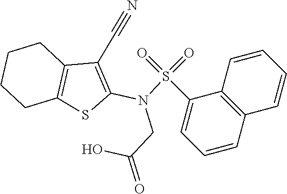 [SMILES: N#Cc1c(N(CC(=O)O)S(=O)(=O)c2cccc3ccccc23)sc2c1CCCC2] | | X |
| PKL-A61 | 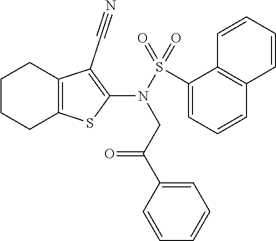 [SMILES: N#Cc1c(N(CC(=O)c2ccccc2)S(=O)(=O)c2cccc3ccccc23)sc2c1CCCC2] | | X |
| PKL-A62 | 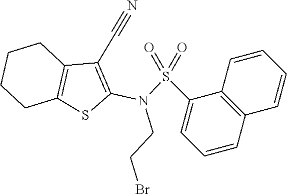 [SMILES: N#Cc1c(N(CCBr)S(=O)(=O)c2cccc3ccccc23)sc2c1CCCC2] | | X |
| PKL-A55 | 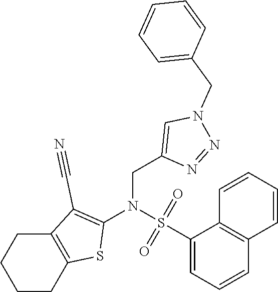 [SMILES: N#Cc1c(N(Cc2cn(Cc3ccccc3)nn2)S(=O)(=O)c2cccc3ccccc23)sc2c1CCCC2] | | X |
| PKL-A64 | 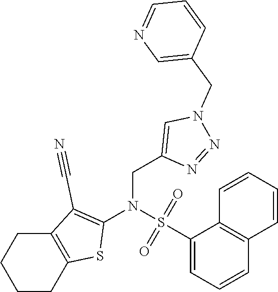 [SMILES: N#Cc1c(N(Cc2cn(Cc3cccnc3)nn2)S(=O)(=O)c2cccc3ccccc23)sc2c1CCCC2] | | X |
| PKL-A48-1 | 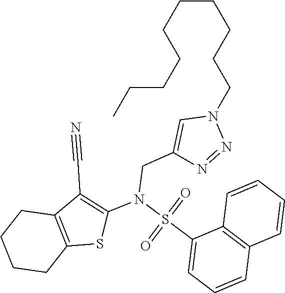 [SMILES: CCCCCCCCCCn1cc(CN(c2sc3c(c2C#N)CCCC3)S(=O)(=O)c2cccc3ccccc23)nn1] | | X |
| PKL-B1 | 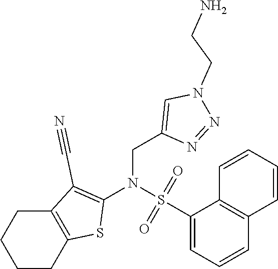 [SMILES: N#Cc1c(N(Cc2cn(CCN)nn2)S(=O)(=O)c2cccc3ccccc23)sc2c1CCCC2] | | X |
| PKL-A73 | 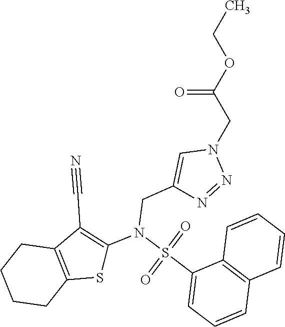 [SMILES: CCOC(=O)Cn1cc(CN(c2sc3c(c2C#N)CCCC3)S(=O)(=O)c2cccc3ccccc23)nn1] | | X |
| PKL-A67 | 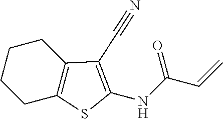 [SMILES: C=CC(=O)Nc1sc2c(c1C#N)CCCC2] | | X |
| PKL-A69 | 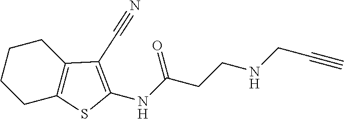 [SMILES: C#CCNCCC(=O)Nc1sc2c(c1C#N)CCCC2] | | X |
| PKL-A68 | 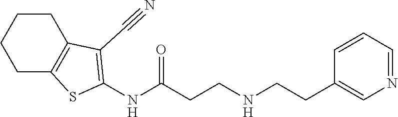 [SMILES: N#Cc1c(NC(=O)CCNCCc2cccnc2)sc2c1CCCC2] | | X |
| PKL-A72 | 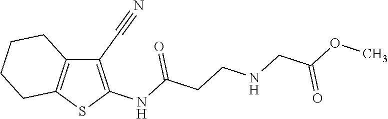 [SMILES: COC(=O)CNCCC(=O)Nc1sc2c(c1C#N)CCCC2] | | X |
| PKL-A70 | 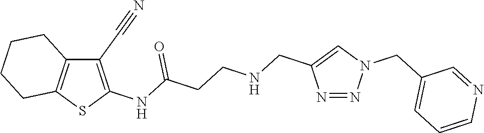 [SMILES: N#Cc1c(NC(=O)CCNCc2cn(Cc3cccnc3)nn2)sc2c1CCCC2] | | X |
| PKL-A1 | 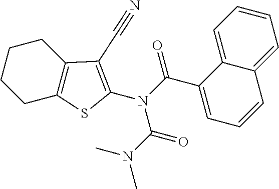 [SMILES: CN(C)C(=O)N(C(=O)c1cccc2ccccc12)c1sc2c(c1C#N)CCCC2] | | X |
| PKL-B28 | 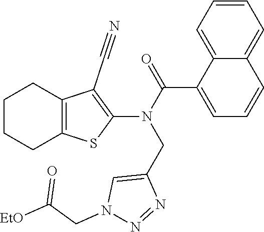 [SMILES: CCOC(=O)Cn1cc(CN(C(=O)c2cccc3ccccc23)c2sc3c(c2C#N)CCCC3)nn1] | | X |
| PKL-B30 | 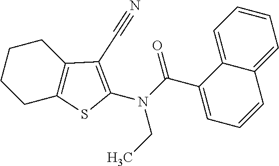 [SMILES: CCN(C(=O)c1cccc2ccccc12)c1sc2c(c1C#N)CCCC2] | | X |
| PKL-B31 | 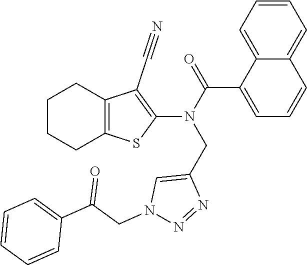 [SMILES: N#Cc1c(N(Cc2cn(CC(=O)c3ccccc3)nn2)C(=O)c2cccc3ccccc23)sc2c1CCCC2] | | X |
| PKL-B32 | 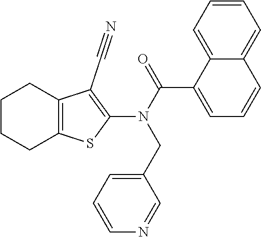 [SMILES: N#Cc1c(N(Cc2cccnc2)C(=O)c2cccc3ccccc23)sc2c1CCCC2] | | X |
| PKL-B33 | 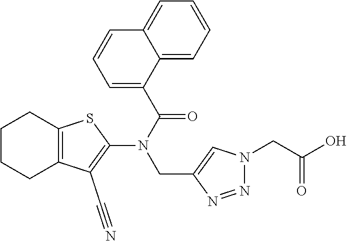 [SMILES: N#Cc1c(N(Cc2cn(CC(=O)O)nn2)C(=O)c2cccc3ccccc23)sc2c1CCCC2] | | X |
| PKL-B35 | 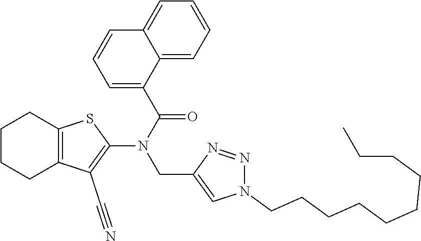 [SMILES: CCCCCCCCCCn1cc(CN(C(=O)c2cccc3ccccc23)c2sc3c(c2C#N)CCCC3)nn1] | | X |
| PKL-B4 | 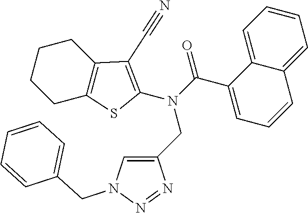 [SMILES: N#Cc1c(N(Cc2cn(Cc3ccccc3)nn2)C(=O)c2cccc3ccccc23)sc2c1CCCC2] | | X |
| PKL-B40 | 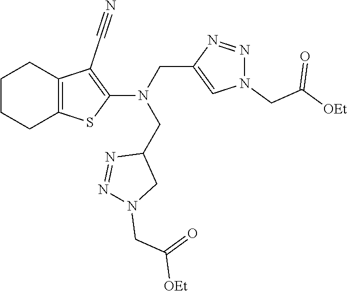 [SMILES: CCOC(=O)CN1CC(CN(Cc2cn(CC(=O)OCC)nn2)c2sc3c(c2C#N)CCCC3)N=N1] | | X |
| PKL-B36 | 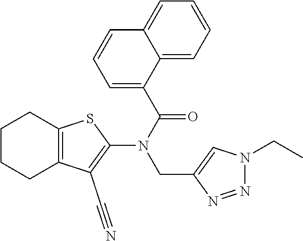 [SMILES: CCn1cc(CN(C(=O)c2cccc3ccccc23)c2sc3c(c2C#N)CCCC3)nn1] | | X |
| PKL-B19/ PKL-A131 | 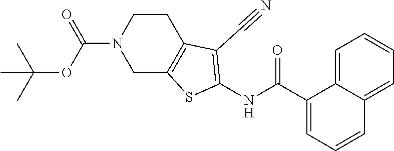 [SMILES: CC(C)(C)OC(=O)N1CCc2c(sc(NC(=O)c3cccc4ccccc34)c2C#N)C1] | X | |
| PKL-A15 | 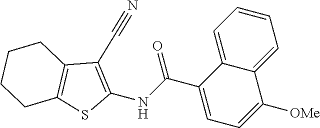 [SMILES: COc1ccc(C(=O)Nc2sc3c(c2C#N)CCCC3)c2ccccc12] | | X |
| PKL-A79 | 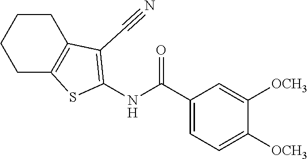 [SMILES: COc1ccc(C(=O)Nc2sc3c(c2C#N)CCCC3)cc1OC] | | X |
| PKL-A102 | 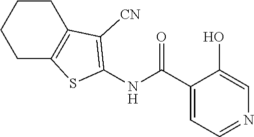 [SMILES: N#Cc1c(NC(=O)c2ccncc2O)sc2c1CCCC2] | | X |
| PKL-A117 | 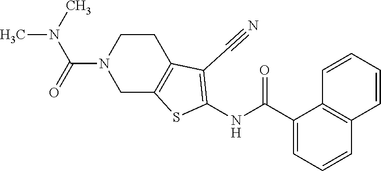 [SMILES: CN(C)C(=O)N1CCc2c(sc(NC(=O)c3cccc4ccccc34)c2C#N)C1] | X | |
| PKL-A119 | 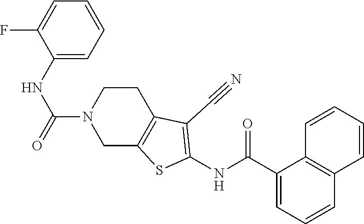 [SMILES: N#Cc1c(NC(=O)c2cccc3ccccc23)sc2c1CCN(C(=O)Nc1ccccc1F)C2] | X | |
| PKL-A130 | 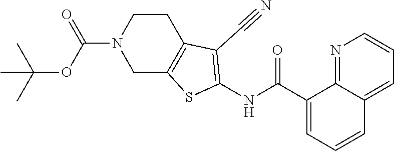 [SMILES: CC(C)(C)OC(=O)N1CCc2c(sc(NC(=O)c3cccc4cccnc34)c2C#N)C1] | X | |
| PKL-A131/ PKL-B19 | 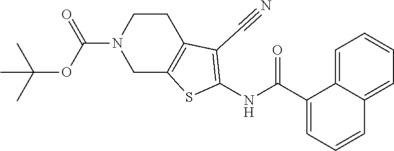 [SMILES: CC(C)(C)OC(=O)N1CCc2c(sc(NC(=O)c3cccc4ccccc34)c2C#N)C1] | X | |
| PKL-A132 | 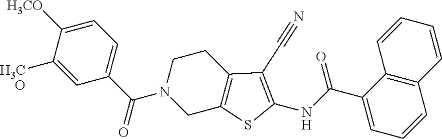 [SMILES: COc1ccc(C(=O)N2CCc3c(sc(NC(=O)c4cccc5ccccc45)c3C#N)C2)cc1OC] | X | |
| PKL-A133 | 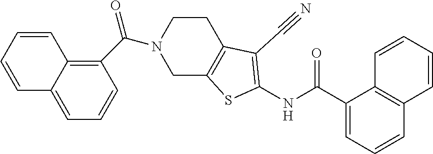 [SMILES: N#Cc1c(NC(=O)c2cccc3ccccc23)sc2c1CCN(C(=O)c1cccc3ccccc13)C2] | X | |
| PKL-A121 | 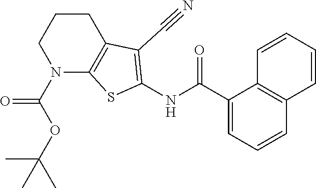 [SMILES: CC(C)(C)OC(=O)N1CCCc2c1sc(NC(=O)c1cccc3ccccc13)c2C#N] | | X |
| PKL-A128 | 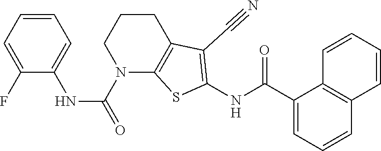 [SMILES: N#Cc1c(NC(=O)c2cccc3ccccc23)sc2c1CCCN2C(=O)Nc1ccccc1F] | | X |
| PKL-142 | 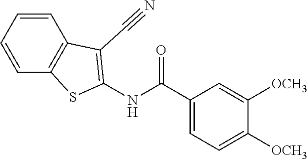 [SMILES: COc1ccc(C(=O)Nc2sc3ccccc3c2C#N)cc1OC] | | X |
| PKL-B74 | 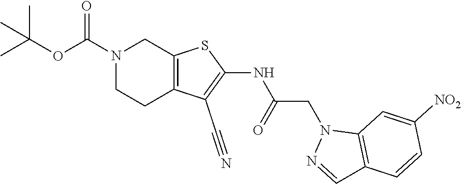 [SMILES: CC(C)(C)OC(=O)N1CCc2c(sc(NC(=O)Cn3ncc4ccc([N+](=O)[O-])cc43)c2C#N)C1] | | X |
| PKL-B70 | 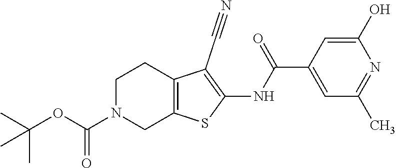 [SMILES: Cc1cc(C(=O)Nc2sc3c(c2C#N)CCN(C(=O)OC(C)(C)C)C3)cc(O)n1] | | X |
| PKL-B55 | 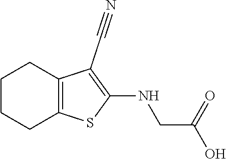 [SMILES: N#Cc1c(NCC(=O)O)sc2c1CCCC2] | | X |
| PKL-B79 | 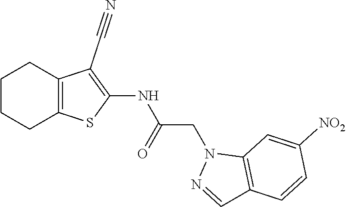 [SMILES: N#Cc1c(NC(=O)Cn2ncc3ccc([N+](=O)[O-])cc32)sc2c1CCCC2] | | X |
| PKL-B73 | 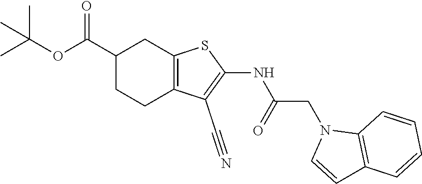 [SMILES: CC(C)(C)OC(=O)C1CCc2c(sc(NC(=O)Cn3ccc4ccccc43)c2C#N)C1] | | X |
| PKL-B78 | 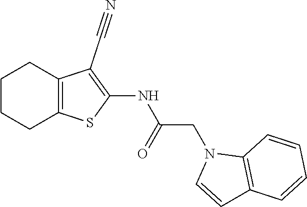 [SMILES: N#Cc1c(NC(=O)Cn2ccc3ccccc32)sc2c1CCCC2] | | X |
| PKL-B86 | 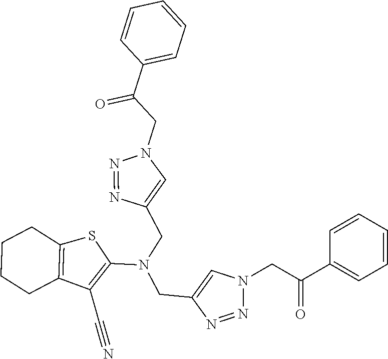 [SMILES: N#Cc1c(N(Cc2cn(CC(=O)c3ccccc3)nn2)Cc2cn(CC(=O)c3ccccc3)nn2)sc2c1CCCC2] | | X |
| PKL-B83 | 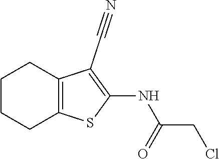 [SMILES: N#Cc1c(NC(=O)CCl)sc2c1CCCC2] | | X |
| PKL-A15 | 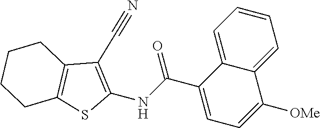 [SMILES: COc1ccc(C(=O)Nc2sc3c(c2C#N)CCCC3)c2ccccc12] | | X |
| PKL-A31 | 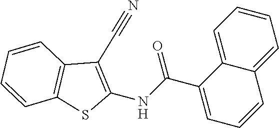 [SMILES: N#Cc1c(NC(=O)c2cccc3ccccc23)sc2ccccc12] | | X |
| PKL-A56 | 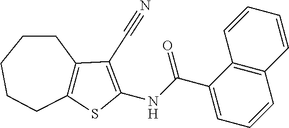 [SMILES: N#Cc1c(NC(=O)c2cccc3ccccc23)sc2c1CCCCC2] | X | |
| PKL-A103 | 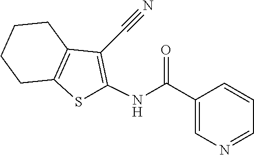 [SMILES: N#Cc1c(NC(=O)c2cccnc2)sc2c1CCCC2] | | X |
| PKL-A114 | 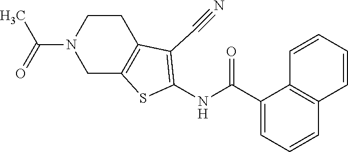 [SMILES: CC(=O)N1CCc2c(sc(NC(=O)c3cccc4ccccc34)c2C#N)C1] | X | |
| PKL-A118 | 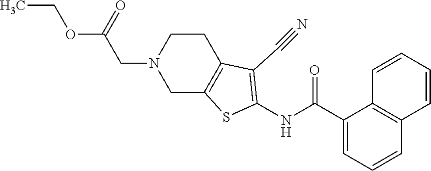 [SMILES: CCOC(=O)CN1CCc2c(sc(NC(=O)c3cccc4ccccc34)c2C#N)C1] | X | |
| PKL-A135 | 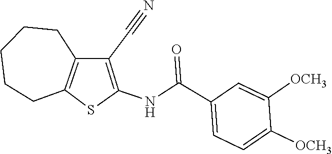 [SMILES: COc1ccc(C(=O)Nc2sc3c(c2C#N)CCCCC3)cc1OC] | X | |
| PKL-A142 | 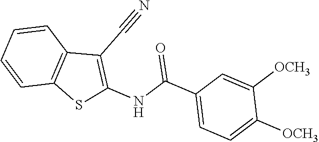 [SMILES: COc1ccc(C(=O)Nc2sc3ccccc3c2C#N)cc1OC] | | X |
| PKL-A149 | 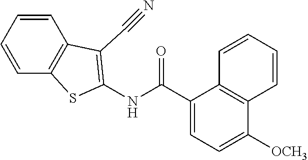 [SMILES: COc1ccc(C(=O)Nc2sc3ccccc3c2C#N)c2ccccc12] | X | |
| PKL-B85 | 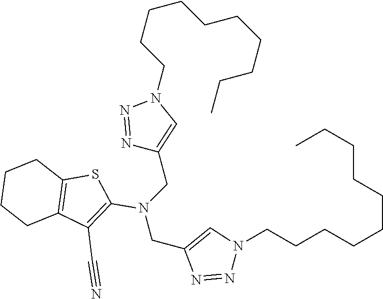 [SMILES: CCCCCCCCCCn1cc(CN(Cc2cn(CCCCCCCCCC)nn2)c2sc3c(c2C#N)CCCC3)nn1] | | X |
| PKL-B52 | 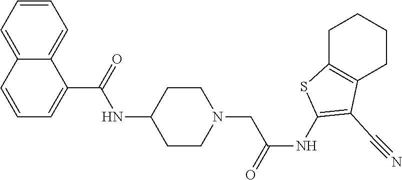 [SMILES: N#Cc1c(NC(=O)CN2CCC(NC(=O)c3cccc4ccccc34)CC2)sc2c1CCCC2] | | X |
| PKL-B129 | 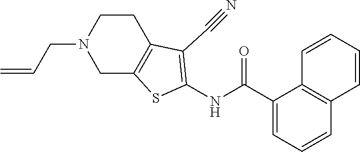 [SMILES: C=CCN1CCc2c(sc(NC(=O)c3cccc4ccccc34)c2C#N)C1] | X | |
| PKL-B125 | 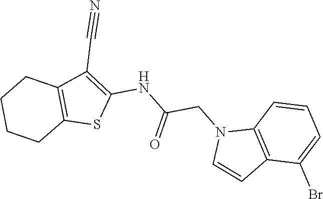 [SMILES: N#Cc1c(NC(=O)Cn2ccc3c(Br)cccc32)sc2c1CCCC2] | | X |
| PKL-B133 | 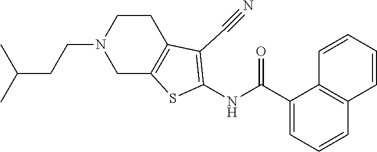 [SMILES: CC(C)CCN1CCc2c(sc(NC(=O)c3cccc4ccccc34)c2C#N)C1] | X | |
| PKL-B124 | 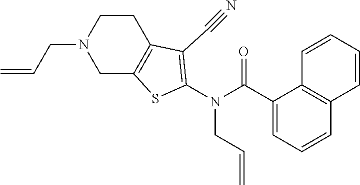 [SMILES: C=CCN1CCc2c(sc(N(CC=C)C(=O)c3cccc4ccccc34)c2C#N)C1] | | X |
| PKL-130 | 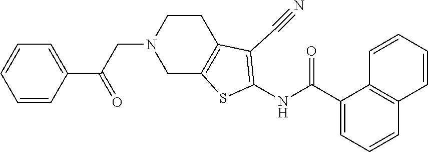 [SMILES: N#Cc1c(NC(=O)c2cccc3ccccc23)sc2c1CCN(CC(=O)c1ccccc1)C2] | X | |
| PKL-B39 | 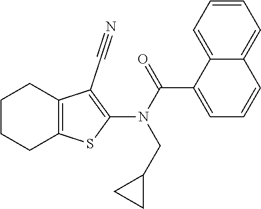 [SMILES: N#Cc1c(N(CC2CC2)C(=O)c2cccc3ccccc23)sc2c1CCCC2] | | X |
| PKL-A151 | 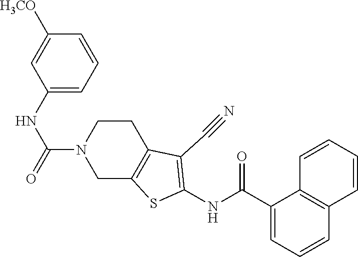 [SMILES: COc1cccc(NC(=O)N2CCc3c(sc(NC(=O)c4cccc5ccccc45)c3C#N)C2)c1] | X | |
| PKL-A152 | 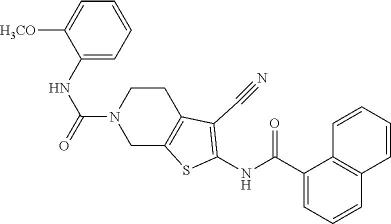 [SMILES: COc1ccccc1NC(=O)N1CCc2c(sc(NC(=O)c3cccc4ccccc34)c2C#N)C1] | X | |
| PKL-A153 | 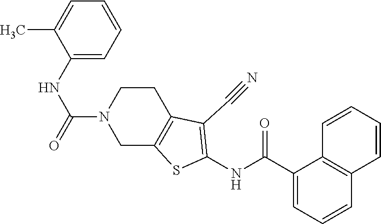 [SMILES: Cc1ccccc1NC(=O)N1CCc2c(sc(NC(=O)c3cccc4ccccc34)c2C#N)C1] | X | |
| PKL-A154 | 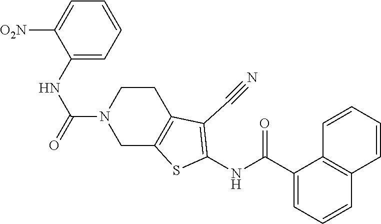 [SMILES: N#Cc1c(NC(=O)c2cccc3ccccc23)sc2c1CCN(C(=O)Nc1ccccc1[N+](=O)[O-])C2] | X | |
| PKL-A161 | 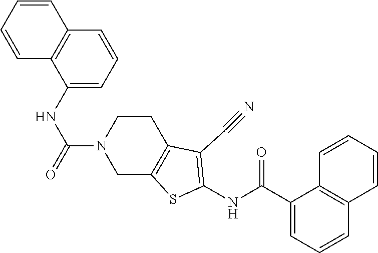 [SMILES: N#Cc1c(NC(=O)c2cccc3ccccc23)sc2c1CCN(C(=O)Nc1cccc3ccccc13)C2] | X | |
| PKL-A162 | 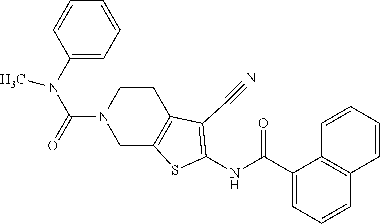 [SMILES: CN(C(=O)N1CCc2c(sc(NC(=O)c3cccc4ccccc34)c2C#N)C1)c1ccccc1] | X | |
| PKL-A163 | 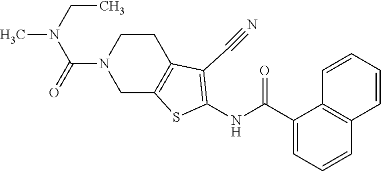 [SMILES: CCN(C)C(=O)N1CCc2c(sc(NC(=O)c3cccc4ccccc34)c2C#N)C1] | X | |
| PKL-A179 | 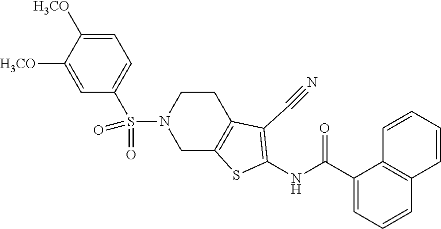 [SMILES: COc1ccc(S(=O)(=O)N2CCc3c(sc(NC(=O)c4cccc5ccccc45)c3C#N)C2)cc1OC] | X | |
| PKL-B126 | 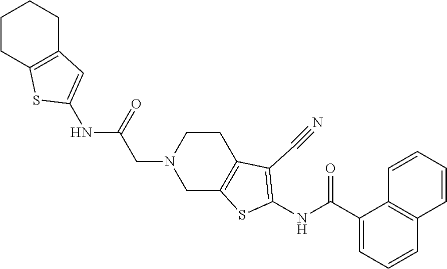 [SMILES: N#Cc1c(NC(=O)c2cccc3ccccc23)sc2c1CCN(CC(=O)Nc1cc3c(s1)CCCC3)C2] | | X |
| PKL-B153 | 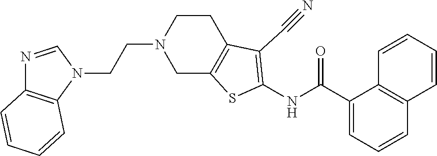 [SMILES: N#Cc1c(NC(=O)c2cccc3ccccc23)sc2c1CCN(CCn1cnc3ccccc31)C2] | X | |
| PKL-B164 | 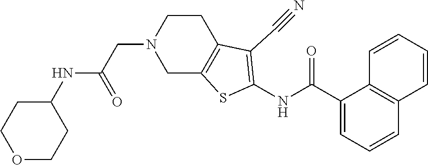 [SMILES: N#Cc1c(NC(=O)c2cccc3ccccc23)sc2c1CCN(CC(=O)NC1CCOCC1)C2] | X | |
| PKL-B171 | 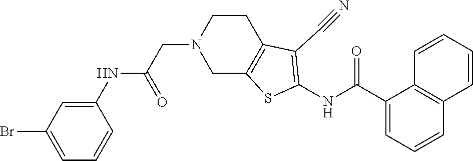 [SMILES: N#Cc1c(NC(=O)c2cccc3ccccc23)sc2c1CCN(CC(=O)Nc1cccc(Br)c1)C2] | X | |
| PKL-B172 | 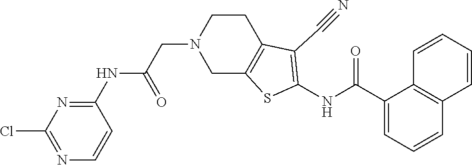 [SMILES: N#Cc1c(NC(=O)c2cccc3ccccc23)sc2c1CCN(CC(=O)Nc1ccnc(Cl)n1)C2] | X | |
| PKL-A156 | 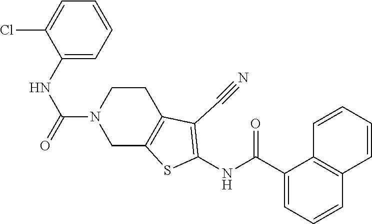 [SMILES: N#Cc1c(NC(=O)c2cccc3ccccc23)sc2c1CCN(C(=O)Nc1ccccc1Cl)C2] | X | |
| PKL-A157 | 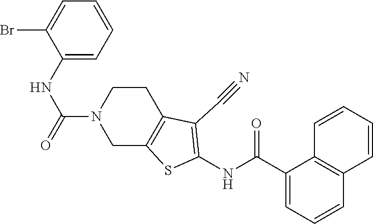 [SMILES: N#Cc1c(NC(=O)c2cccc3ccccc23)sc2c1CCN(C(=O)Nc1ccccc1Br)C2] | X | |
| PKL-A158 | 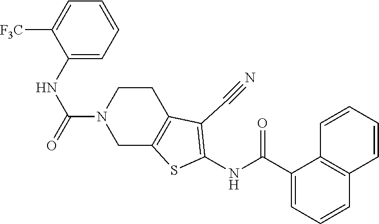 [SMILES: N#Cc1c(NC(=O)c2cccc3ccccc23)sc2c1CCN(C(=O)Nc1ccccc1C(F)(F)F)C2] | X | |
| PKL-A159 | 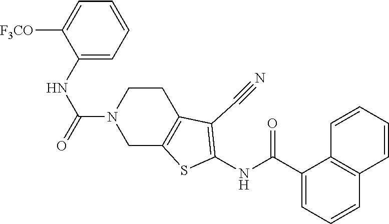 [SMILES: N#Cc1c(NC(=O)c2cccc3ccccc23)sc2c1CCN(C(=O)Nc1ccccc1OC(F)(F)F)C2] | X | |
| PKL-A160 | 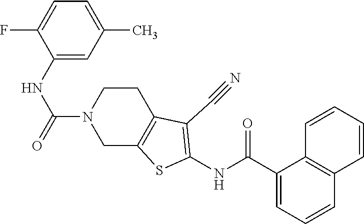 [SMILES: Cc1ccc(F)c(NC(=O)N2CCc3c(sc(NC(=O)c4cccc5ccccc45)c3C#N)C2)c1] | X | |
| PKL-A183 | 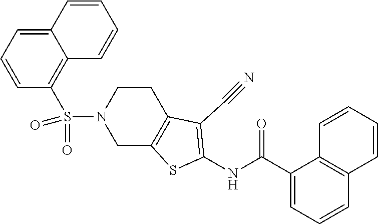 [SMILES: N#Cc1c(NC(=O)c2cccc3ccccc23)sc2c1CCN(S(=O)(=O)c1cccc3ccccc13)C2] | X | |
| PKL-A190 | 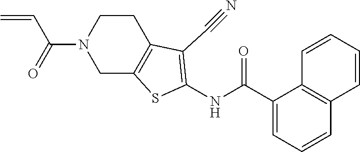 [SMILES: C=CC(=O)N1CCc2c(sc(NC(=O)c3cccc4ccccc34)c2C#N)C1] | X | |
| PKL-A192 | 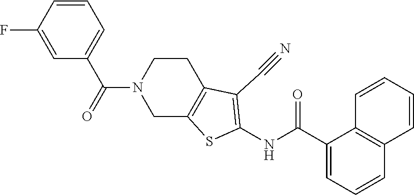 [SMILES: N#Cc1c(NC(=O)c2cccc3ccccc23)sc2c1CCN(C(=O)c1cccc(F)c1)C2] | X | |
| PKL-A193 | 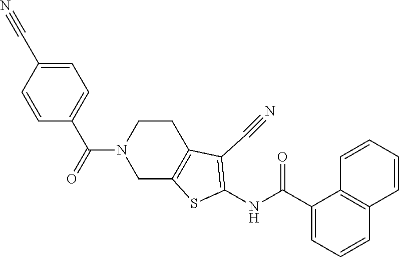 [SMILES: N#Cc1ccc(C(=O)N2CCc3c(sc(NC(=O)c4cccc5ccccc45)c3C#N)C2)cc1] | X | |
| PKL-B165 | 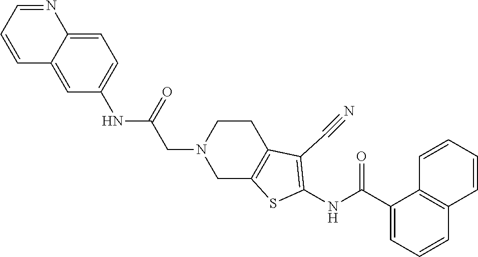 [SMILES: N#Cc1c(NC(=O)c2cccc3ccccc23)sc2c1CCN(CC(=O)Nc1ccc3ncccc3c1)C2] | | X |
| PKL-B168 | 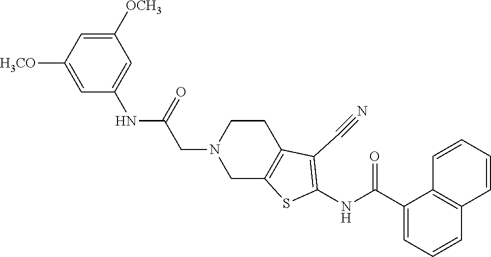 [SMILES: COc1cc(NC(=O)CN2CCc3c(sc(NC(=O)c4cccc5ccccc45)c3C#N)C2)cc(OC)c1] | | X |
| PKL-B169 | 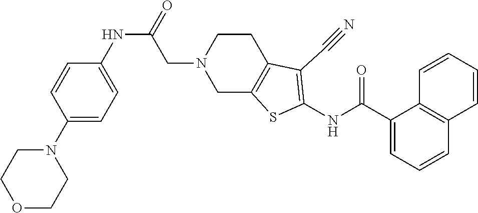 [SMILES: N#Cc1c(NC(=O)c2cccc3ccccc23)sc2c1CCN(CC(=O)Nc1ccc(N3CCOCC3)cc1)C2] | | X |
| PKL-B170 | 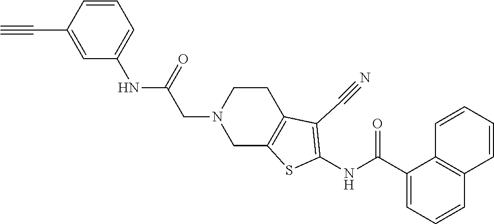 [SMILES: C#Cc1cccc(NC(=O)CN2CCc3c(sc(NC(=O)c4cccc5ccccc45)c3C#N)C2)c1] | X | |
| PKL-B173 | 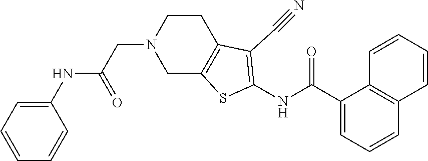 [SMILES: N#Cc1c(NC(=O)c2cccc3ccccc23)sc2c1CCN(CC(=O)Nc1ccccc1)C2] | X | |
| PKL-B174 | 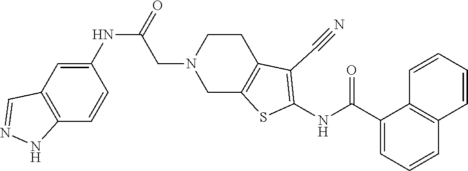 [SMILES: N#Cc1c(NC(=O)c2cccc3ccccc23)sc2c1CCN(CC(=O)Nc1ccc3[nH]ncc3c1)C2] | | X |
| PKL-B176 | 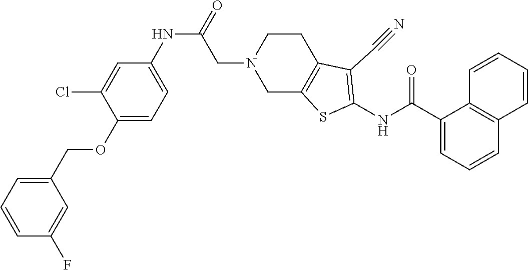 [SMILES: N#Cc1c(NC(=O)c2cccc3ccccc23)sc2c1CCN(CC(=O)Nc1ccc(OCc3cccc(F)c3)c(Cl)c1)C2] | | X |

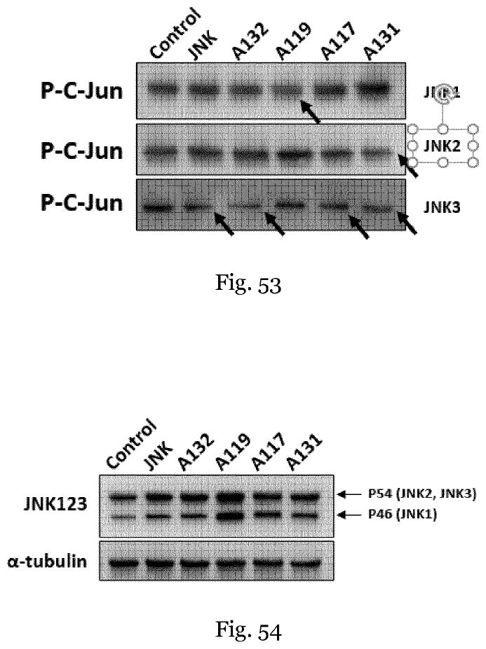

The JNK enzyme activity test, see FIG. 52-53, showed that the tested target molecules (A132, A119, A117 and A131) inhibited of expression of JNK1, JNK2 and JNK3. The greatest inhibition was obtained for JNK3.

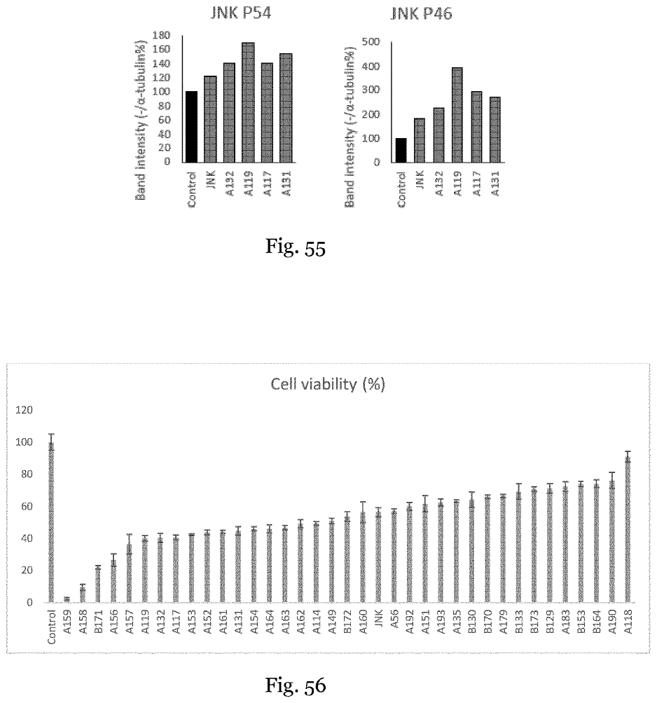

The protein-ligand binding was assessed for selected drug candidates, see FIG. 54-55. The tested drug candidates (A132, A119, A117 and A131) exhibited a better binding to the JNK protein than JNK-IN-5A.

### Ranking of the Target Compounds

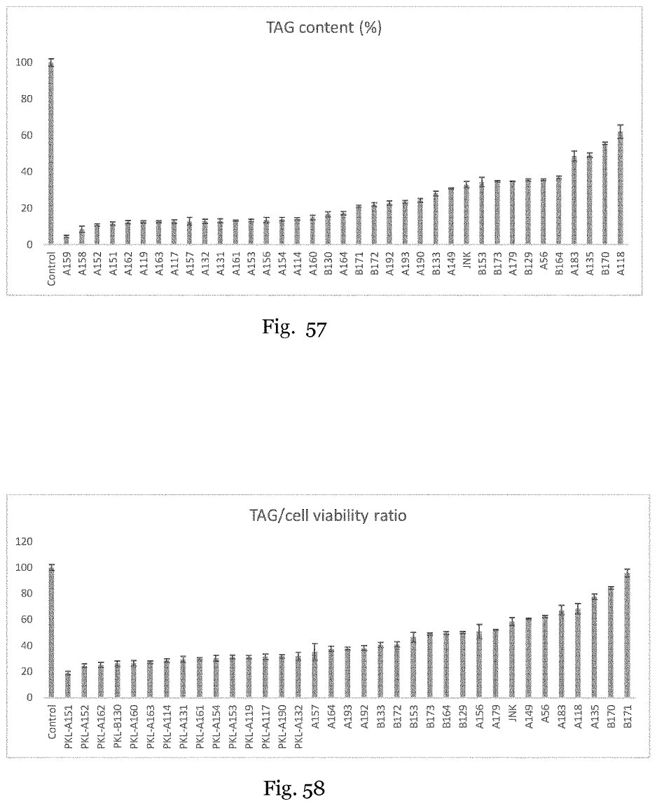

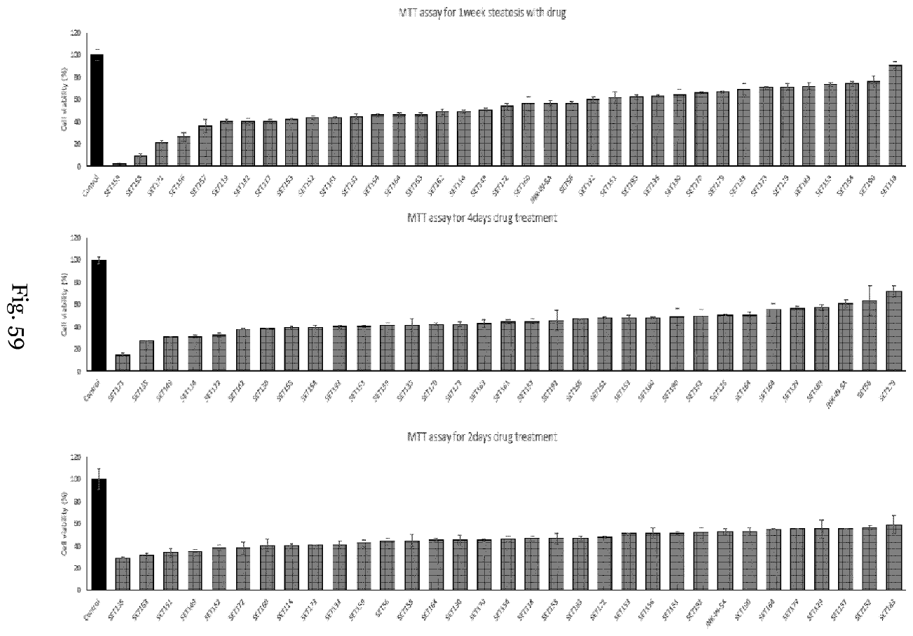

The ranking of the synthesized target compounds can be seen in Tables 2-4. The ranking is based on TAG contents, cell viability and the ratio between TAG content and cell viability. The ranking based on cell viability, Table 3 and FIG. 56, shows the compounds that may be suitable for treatment of HCC where a low cell viability is desired. The rankings based on TAG content and the ratio between TAG content and cell viability, Tables 2 and 4 and FIG. 57-58, show the compounds which are most suitable for treatment of fatty liver disease where a low TAG content together with a low toxicity is desired.

PKL-A159 and PKL-A158 had the greatest effect on TAG accumulation, having a TAG content of 4.54% and 8.35% respectively, see Table 2 and FIG. 57. These compounds were followed by PKL-A152 and PKL-A151 having a TAG content of 10.77% and 11.51%. 26 of the tested target compounds resulted in lower TAG content than JNK-IN-5A.

TABLE 2
Ranking of the tested target compounds based on TAG content.

| Ranking | Compound | TAG (%) | SD± |
|---|---|---|---|
| — | Control | 100.00 | 2.16 |
| 1 | A159 | 4.54 | 0.75 |
| 2 | A158 | 8.35 | 1.71 |
| 3 | A152 | 10.77 | 0.67 |
| 4 | A151 | 11.51 | 0.90 |
| 5 | A162 | 12.35 | 0.96 |
| 6 | A119 | 12.54 | 0.58 |
| 7 | A163 | 12.68 | 0.50 |
| 8 | A117 | 12.75 | 0.90 |
| 9 | A157 | 12.80 | 2.25 |
| 10 | A132 | 12.85 | 1.18 |
| 11 | A131 | 13.10 | 1.03 |
| 12 | A161 | 13.15 | 0.38 |
| 13 | A153 | 13.25 | 0.68 |
| 14 | A156 | 13.54 | 1.49 |
| 15 | A154 | 13.94 | 0.99 |
| 16 | A114 | 14.11 | 0.69 |
| 17 | A160 | 14.87 | 1.27 |
| 18 | B130 | 16.76 | 1.28 |
| 19 | A164 | 17.28 | 0.91 |
| 20 | B171 | 21.05 | 0.69 |
| 21 | B172 | 21.97 | 1.10 |
| 22 | A192 | 22.85 | 1.16 |
| 23 | A193 | 23.41 | 0.74 |
| 24 | A190 | 24.22 | 1.11 |
| 25 | B133 | 28.11 | 1.33 |
| 26 | A149 | 30.78 | 0.37 |
| 27 | JNK | 32.97 | 1.83 |
| 28 | B153 | 34.34 | 2.63 |
| 29 | B173 | 34.71 | 0.48 |
| 30 | A179 | 34.72 | 0.18 |
| 31 | B129 | 35.52 | 0.58 |
| 32 | A56 | 35.59 | 0.44 |
| 33 | B164 | 36.79 | 0.75 |
| 34 | A183 | 48.61 | 2.75 |
| 35 | A135 | 49.06 | 1.25 |
| 36 | B170 | 55.63 | 0.72 |
| 37 | A118 | 62.07 | 3.72 |

Treatment with PKL-A159 and PKL-A158 resulted in the greatest reduction of ell viability and were hence the most toxic compounds, see Table 3 and FIG. 6. The cell viability of these compounds was 2.33% and 9.59% respectively. Treatment with PKL-A159 and PKL-A158 were followed by treatment with PKL-B171 and PKL-A156, which resulted in higher cell viability, 22.00% and 26.64% respectively, but were still toxic. These four compounds and in particular PKL-A159, PKL-A158 and PKL-B171, may be particularly suitable for treatment of HCC where a great reductions of cell viability is desired.

TABLE 3
Ranking of the tested target compounds based on cell viability.

| Ranking | Compound | Cell viability (%) | SD± |
|---|---|---|---|
| — | Control | 100.00 | 5.02 |
| 1 | A159 | 2.33 | 0.84 |
| 2 | A158 | 9.59 | 1.96 |
| 3 | B171 | 22.00 | 1.18 |
| 4 | A156 | 26.64 | 3.83 |
| 5 | A157 | 36.47 | 6.01 |
| 6 | A119 | 40.12 | 1.57 |
| 7 | A132 | 40.31 | 2.91 |
| 8 | A117 | 40.59 | 1.58 |
| 9 | A153 | 42.50 | 0.43 |
| 10 | A152 | 43.87 | 1.42 |
| 11 | A161 | 44.06 | 0.93 |
| 12 | A131 | 44.75 | 2.57 |
| 13 | A154 | 45.95 | 1.38 |
| 14 | A164 | 46.01 | 2.47 |
| 15 | A163 | 46.64 | 1.44 |
| 16 | A162 | 49.20 | 2.63 |
| 17 | A114 | 49.35 | 1.28 |
| 18 | A149 | 50.88 | 1.66 |
| 19 | B172 | 53.70 | 3.04 |
| 20 | A160 | 56.41 | 6.36 |
| 21 | JNK | 56.44 | 2.85 |
| 22 | A56 | 56.98 | 1.47 |
| 23 | A192 | 60.10 | 2.44 |
| 24 | A151 | 61.60 | 5.00 |
| 25 | A193 | 62.32 | 2.39 |
| 26 | A135 | 63.24 | 0.68 |
| 27 | B130 | 64.10 | 4.88 |
| 28 | B170 | 66.03 | 1.06 |
| 29 | A179 | 66.54 | 0.97 |
| 30 | B133 | 69.29 | 4.79 |
| 31 | B173 | 70.83 | 1.40 |
| 32 | B129 | 71.23 | 3.01 |
| 33 | A183 | 72.28 | 3.00 |
| 34 | B153 | 74.00 | 1.70 |
| 35 | B164 | 74.13 | 2.45 |
| 36 | A190 | 76.16 | 5.12 |
| 37 | A118 | 91.00 | 3.20 |

PKL-A159 and PKL-A158 were removed from the ranking of the ratio between TAG content and cell viability due to their high toxicity which would not be suitable in the treatment of fatty liver disease. PKL-A151 exhibits the lowest ratio between TAG/MTT, see Table 4 and FIG. 58, and is hence particularly suitable for the treatment of fatty liver disease. Treatment with PKL-A151 resulted in a TAG content/cell viability ratio of 18.7 which is more than 3 times lower than JNK-IN-5A while having a higher cell viability than JNK-IN-5A. Treatment with PKL-A151 is followed by treatment with PKL-A152 and PKL-A162 as top drug candidates for the treatment of fatty liver disease resulting in a TAG/cell viability of 24.5% and 25.1% respectively. 27 of the tested compounds exhibited a lower TAG content than JNK-IN-5A.

TABLE 4
Ranking of the tested target compounds based on
the ratio between TAG content and cell viability.

| Ranking | Compound | TAG/cell viability | SD± |
|---|---|---|---|
| — | Control | 100.0 | 2.2 |
| 1 | A151 | 18.7 | 1.5 |
| 2 | A152 | 24.5 | 1.5 |
| 3 | A162 | 25.1 | 2.0 |
| 4 | B130 | 26.2 | 2.0 |
| 5 | A160 | 26.4 | 2.2 |
| 6 | A163 | 27.2 | 1.1 |
| 7 | A114 | 28.6 | 1.4 |
| 8 | A131 | 29.3 | 2.3 |
| 9 | A161 | 29.8 | 0.9 |
| 10 | A154 | 30.3 | 2.1 |
| 11 | A153 | 31.2 | 1.6 |
| 12 | A119 | 31.2 | 1.4 |
| 13 | A117 | 31.4 | 2.2 |
| 14 | A190 | 31.8 | 1.5 |
| 15 | A132 | 31.9 | 2.9 |
| 16 | A157 | 35.1 | 6.2 |
| 17 | A164 | 37.6 | 2.0 |
| 18 | A193 | 37.6 | 1.2 |
| 19 | A192 | 38.0 | 1.9 |
| 20 | B133 | 40.6 | 1.9 |
| 21 | B172 | 40.9 | 2.1 |
| 22 | B153 | 46.4 | 3.6 |
| 23 | B173 | 49.0 | 0.7 |
| 24 | B164 | 49.6 | 1.0 |
| 25 | B129 | 49.9 | 0.8 |
| 26 | A156 | 50.8 | 5.6 |
| 27 | A179 | 52.2 | 0.3 |
| 28 | JNK | 58.4 | 3.2 |
| 29 | A149 | 60.5 | 0.7 |
| 30 | A56 | 62.5 | 0.8 |
| 31 | A183 | 67.2 | 3.8 |
| 32 | A118 | 68.2 | 4.1 |
| 33 | A135 | 77.6 | 2.0 |
| 34 | B170 | 84.2 | 1.1 |
| 35 | B171 | 95.7 | 3.1 |

In conclusion, several promising candidate drugs have been developed the use in the treatment of fatty liver disease (in particular PKL-A151, PKL-A152, PKL-A162 and PKL-B130) and HCC (PKL-B171). The candidate drugs result in significant reduction of the expression levels of PKLR and other steatosis-related proteins as well as reduced TAG contents.

### Example 2

In this examples, the anti-HCC effect of treatment with JNK-IN-5A and the target compounds PKL-A135, PKL-A156, PKL-A158, PKL-A159, PKL-B171, and PKL-B172 on the HepG2 human HCC cell line is demonstrated. In this example and the corresponding figures, the derivatives will be denoted as SET135 (PKL-A135), SET156 (PKL-A156), SET158 (PKL-A158), SET159 (PKL-A159), SET171 (PKL-B171), and SET172 (PKL-B172).

### Methods

### Cell Culture, Cell Viability and Cytotoxicity Assay.

HepG2 cells were maintained and used with RPMI 1640 (R2405, Sigma-Aldrich) supplemented with 10% fetal bovine serum (F7524, Sigma-Aldrich), 1% P/S (P4333, Sigma-Aldrich). 20,000 cells per well of HepG2 cells were seeded into a 96-well plate and triplicated for MTT assay and LDH assay. Cell viability was measured by MTT assay (M6494, ThermoFisher) and cytotoxicity was measured by LDH assay kit (ab65393, abcam) by following manufacturer's instructions. Optical density (O.D) was numerated using a microplate reader (Hidex Sense Meta Plus).

### Western Blot Analysis

HepG2 cells were seeded into a 6-well plate at 400,000 cells per well. After treatment with 10 μM of JNK-IN-5A and its derivatives for two days, lysates were harvested with CelLytic M (C2978, Sigma-Aldrich). 20 μg lysate was prepared with 2× Laemmli Sample Buffer (1610737, Biorad). SDS PAGE were performed using Mini-PROTEAN® TGX™ Precast Gels (Bio-Rad) and transferred using Trans-Blot® Turbo™ Transfer System (Bio-Rad). Primary antibody, PARP (95425, Cell signalling), Caspase 3 (ab32042, abcam), Bcl-2 (ab182858), STAT1 (HPA000982, Merk), PKL (06653, Sigma), PKM (4053S, Cell signalling), GAPDH (ab8245, abcam), P53 (ab32389, abcam), p21 (ab109199, abcam), p-S349 p62 (ab211324, abcam), p62 (ab109012, abcam), c-Myc (ab32072, abcam), GPX4 (ab125066, abcam), MMP9 (MA5-32705, Invitrogen), β-actin (ab8227, abcam), and α-tubulin (ab7291, abcam) were blotted overnight and Goat Anti-Rabbit HRP (ab205718) and goat anti-mouse IgG-HRP (sc2005, Santa Cruz Biotechnology, Inc.) were blotted for secondary antibody for one hour. ImageQuantTMLAS 500 (29-0050-63, GE) was used to detect protein bands.

### FACs Analysis

400,000 HepG2 cells were seeded in a 6-well plated and treated with 10 μM of the compounds for 2 days. Cells were fixed with 70% ethanol and stained with 50 μg/ml PI. Prepared cells were analysed using BD FACS Canto II Flow Cytometry system at PE-A channel 10,000 cells per group. The data was histogram plotted with BD FACSDiva 9.0.

### Mitochondria Membrane Potential, Autophagy, ROS, and Lipid Peroxidation Assay

HepG2 cells were seeded into a 6-well plate at 400,000 cells per well. After treatment with the compounds at desired concentration and time, TMRE (ab113852, abcam), Autophagy (ab139484, abcam), ROS (ab113851, abcam), and Lipid peroxidation (ab243377, abcam) assay were performed according to manufacturer's instructions. Fluorescence images were taken using ZOE™ Fluorescent Cell Imager (Biorad) and ROI of fluorescence intensity was measured and calculated by Image J. For microplate reader florescence assay, 40,000 of HepG2 cells were seeded into a 96-well plate and triplicated. GFP (Ex480 nm, Em530 nm) and RFP (Ex549 nm, Em574 nm) were measured using a microplate reader (Hidex Sense Meta Plus).

### Invasion Assay and Wound Healing Assay

The effects of the treatment with the compounds on the invasion ability of HepG2 and Huh7 cells were determined by 8 μm pores transwell chamber (Corning™ 3464, USA).

Transwell membrane was coated with 100 μl collagen (Sigma C3867, USA) diluted with PBS to a concentration of 1 mg/ml. After overnight incubation at 4° C., the collagen solution was discarded and the membrane was washed with PBS. 100,000 cells in 200 μl serum free media together with the tested compounds were added into the upper well and 600 μl of 10% FBS supplemented growth media was added to the lower well as an invasive attractant in a 24-well plate. Cells were incubated at 37° C. in a CO₂ incubator for one day. The cells were fixed with 3.7% formaldehyde for 30 minutes at room temperature and washed with PBS twice. The cells were stained with 1% crystal violet in PBS for 20 minutes and residual cells in inner membrane were swabbed gently with a cotton swab. Invaded cells located at the outer membrane were imaged by ZOE Fluorescent Cell Imager (Bio-Rad, USA) and counted by image. For wound healing assay, 100,000 cells per well were seeded into 24-well plates and grown until 95-100% confluent. Linear wound was created by using a 200 μl pipette tip. Image was recorded at the time point of day 0 until control group wound closure with 1 μM of compounds by ZOE Fluorescent Cell Imager (Bio-Rad, USA). Wound areas were measured using ImageJ software.

### RNA Extraction and Sample Preparation for RNA Sequencing

RNA samples for RNA sequencing were extracted with RNeasy Plus Mini Kit (74134, QIAGEN). RIN score of total RNA samples was measured using RNA 6000 Nano kit (5067-1511, Agilent). 2100 Bioanalyzer Instrument (G2939BA, Agilent) was used for RIN score test using electrophoresis. Samples with a Rof IN score>8 were submitted to SZA Omics, Istanbul for RNA sequencing analysis.

### RNA Sequencing

The quality, quantity, and RNA integrity number (RIN) of the samples were assessed with NanoDrop (Thermo Fisher, USA), and Bioanalyzer (2100, Agilent Technology, USA). For the RNA samples, the ratio of absorbance at 260 and 280 nm (A260/280) of −2, A260/230 of 2.0-2.2, and RIN>8.0 are acceptable. All samples passed the quality control.

The Illumina Stranded Total RNA Prep, Ligation with Ribo-Zero Plus kit was used for the construction of NGS libraries from the samples with acceptable quality parameters. In short, RNA samples were sequenced with 2×100 paired-end reads by the NovaSeq 6000 system. Raw sequencing data (.bcl) was demultiplexed and converted to FASTQ files with DRAGEN Software (v3.9.5). The data was delivered in FASTQ format using Illumina 1.8 quality scores.

### RNA-Seq Data Preprocessing

The quality of FASTQ files was assessed by FastQC (v0.11.9) software to ensure the quality of every sequenced sample. Kallisto (v0.48.0)16 was used to quantify the count and transcripts per million (TPM) values of transcripts based on the Homo sapiens reference cDNA (version GRCh38, Ensembl release 110) [16]. The transcript read counts and TPM values were assembled to gene level using the R package tximport (v1.28.0) [17], with only protein-coding transcripts and genes included. The lowly expressed genes with an average count below 5 were filtered out, resulting in a total of 14,592 genes for the downstream analysis.

### RNA-Seq Data Analyses

### Differential Expression Analysis

Differential expression analysis was conducted using the R package DESeq2 (v1.40.2) [18] followed by the Benjamini-Hochberg procedure to correct p-values. Adjusted p-value<0.05 was chosen as the threshold for the significance of differentially expressed genes (DEGs), with log 2 fold change>1 for up-regulation and log 2 fold change<−1 for down-regulation.

### Principal Component Analysis

Principal component analysis (PCA) was applied to visualize the distribution samples, which was performed by the R package pcaMethods (v1.92.0) [19] based on the gene expression profiles after variance stabilizing transformation by DESeq2.

### Gene Set Functional Analysis

Gene set overrepresentation analysis (GSOA) was applied to determine whether a list of DEGs of interest was significantly associated with specific Gene Ontology (GO) biological process terms in the RNA-seq dataset, with all 14,592 analysed genes as the background. In addition, gene set enrichment analysis (GSEA) [20] was performed to assess the activation or inhibition of biological pathways in response to the treatments. Based on this method, genes were sorted based on the log 2 fold change in descending order, and the 50 hallmark gene sets from the Molecular Signatures Database (MSigDB) [21] were tested for their significance. Here, a positive normalized enrichment score (NES) indicates that a hallmark gene set is activated in the corresponding gene list ranked by log 2 fold change, and vice versa. The hallmark gene sets were retrieved from the R package msigdbr (v7.5.1). GSEA was also performed based on the KEGG pathways [22] in the same manner. The R package clusterProfiler (v4.8.2) [ref: 23, 24] was used for both GSOA and GSEA, with p-values adjusted by the Benjamini-Hochberg procedure. An adjusted p-value<0.05 was considered significant.

In addition, the PROGENy analysis [25] was performed to infer the activity scores of the 14 cancer-related pathways. For this, the log 2 fold change values of each treatment vs. control were used as input, and the relative scores across all nine treatments were inferred using the R package progeny (v1.24.0) and decoupleR (v2.8.0) [26].

### Comparison of DEGs

We used three approaches to estimate the similarity between DEGs under different comparisons. First, the Jaccard index (in other words, Jaccard similarity) defined as the ratio of the size of the intersection relative to the size of the union of the two sets of DEGs was calculated. Second, the p-value for the overlap between DEGs by hypergeometric testing was calculated. The p-values were then corrected by the Benjamini-Hochberg procedure. Lastly, the R package GOSemSim (v2.26.1) [27] was used to evaluate the semantic similarity between the significant GO biological process terms that are associated with different sets of DEGs.

### Results

### The Effect of PKL Expression on Cell Viability

The present inventors postulated that JNK-IN-5A and the synthesized target compounds (JNK-IN-5A derivatives) potential use in the treatment of HCC is based on their ability to modulate PKL expression levels. Thus, the inhibition of PKL on cell viability was evaluated.

It was shown that siRNA-mediated inhibition of PKL did not result in the desired anticancer effect (FIG. 84), since WT HepG2 cells also express PKM2. Three distinct siRNAs were transfected into HepG2 cells and, after three days, no statistically significant change in cell viability was observed (FIG. 84). Furthermore, Western blot analysis revealed that the suppression of PKL expression via siRNAs did not result in increased cleaved-PARP or decreased Bcl-2 expression levels, unlike the effects seen with JNK-IN-5A and the synthesized target compounds (FIG. 84B). This suggests the presence of alternative pathways, potentially involving p53 and c-Myc, that lead to apoptosis.

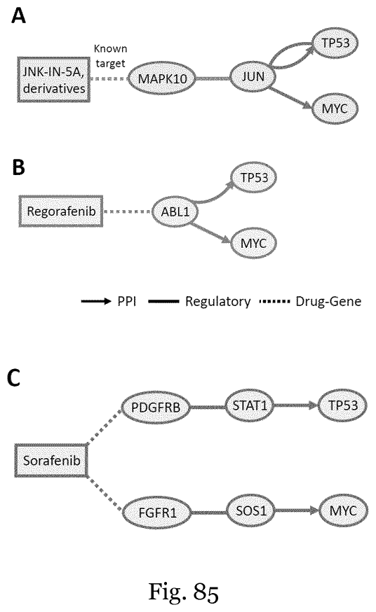

Open MoA is a robust tool for elucidating the mechanism of action (MoA) of drugs by leveraging network topology and hierarchy [28]. Utilizing drug-specific differentially expressed genes (DEGs) data, Open MoA constructs drug-specific subnetworks and identifies the most probable pathways from the drugs to p53 or c-Myc. All identified interactions had a high confidence score, surpassing 0.99 (FIG. 85). For JNK-IN-5A and target compounds, MAPK10, a known target of JNK-IN-5A, was selected as the starting point [14]. JUN was identified as the sole intermediate target in the projected MoA, with its strong association with p53 and c-Myc supported by prior research [29]. Regarding regorafenib, among its 18 direct downstream targets, ABL1 was pinpointed as having the highest likelihood of interacting with p53 and c-Myc. For sorafenib, among its 8 downstream targets, PDGFRB and FGFR1 were deemed the most likely to influence the mechanisms leading to p53 and c-Myc, with STAT1 and SOS1 acting as intermediate proteins, respectively.

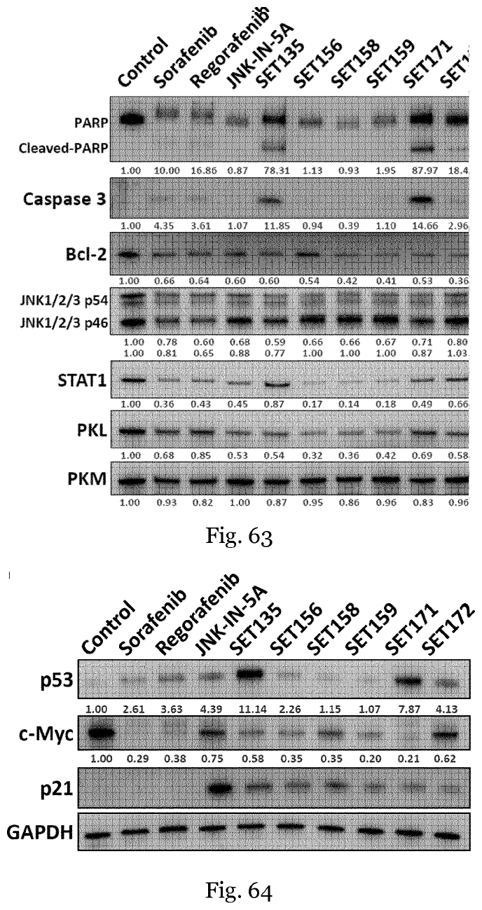

Despite the predicted MoAs for sorafenib, regorafenib, and JNK-IN-5A showing distinct variations, none included PKL, even though all compounds inhibited PKL protein expression (FIG. 63). The intermediate proteins identified within these MoAs may serve as pivotal targets through which the drugs impact apoptosis by modulating p53 and c-Myc activation. The differing MoAs might contribute to the observed variations in cell apoptosis.

### Target Compounds Induce Apoptotic Cell Death, Cell Cycle Arrest and Autophagy Via p53 Tumour Suppressor Pathway

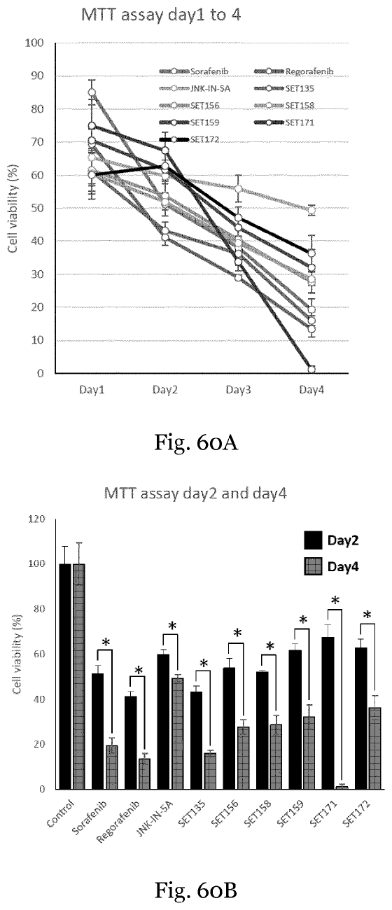

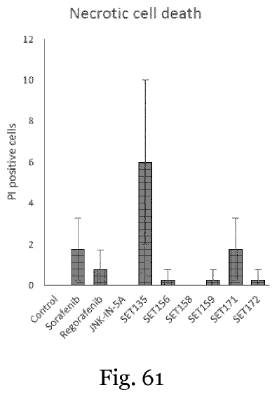

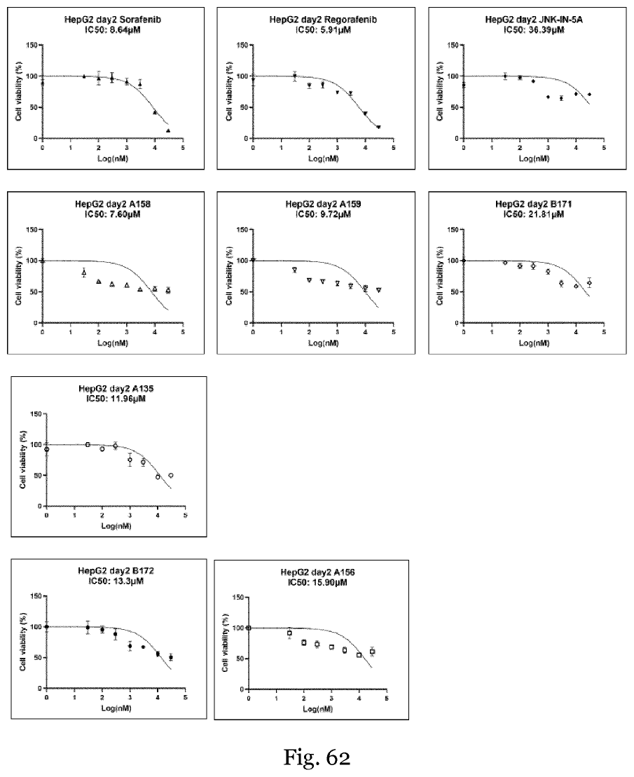

The target compounds were screened for anti-liver cancer effect using MTT assay (FIG. 59). HepG2 cells were treated with 37 different target compounds based on JNK-IN-5A for one week under DNL steatosis condition [30]. Six compounds were selected for their potent toxicity against HepG2 human HCC cell line, including the top four most toxic compounds, SET159, SET158, SET171, and SET156, along with two additional compounds, SET135 and SET172, which demonstrated high toxicity after 2 and 4 days of treatment. The effect of treating HepG2 cells with JNK-IN-5A and the six chosen target compounds was assessed alongside the effect of treatment with established liver cancer drugs regorafenib and sorafenib (FIG. 60A-B). Initially, cell viability was tested using MTT assay at a concentration of 10 μM over a period of up to 4 days (FIG. 60A-B). Treatment with all compounds significantly reduced cell viability after treatment for 2 days: sorafenib (51%±3.6), regorafenib (41.1%±2.4), JNK-IN-5A (59.7%±2.3), SET135 (43.1%±2.7), SET156 (53.7%±4.3), SET158 (51.9%±0.8), SET159 (61.6%±2.9), SET171 (67.4%±5.7), and SET172 (62.7%±4.0) (FIG. 60B). Sorafenib (19.2%±3.4) and regorafenib (13.4%±2.3) demonstrated a time-dependent decrease in cell viability at day 4. Notably, SET171 showed the lowest cell viability at 1.1%±1.1 on day 4, followed by treatment with SET135 which resulted in a cell viability of 15.9%±1.4 (FIG. 60A-B). Both SET135 and SET171 significantly induced necrotic cell death, as evidenced by PI (Propidium Iodide) staining (FIG. 61). The IC50 values of the compounds were determined (FIG. 62 and Table 5).

TABLE 5
The IC50 values of the tested compounds.

| HepG2 | IC50 (μM) |
|---|---|
| Sorafenib | 8.64 |
| Regorafenib | 5.91 |
| JNK-IN-5A | 36.39 |
| SET135 | 11.96 |
| SET156 | 15.90 |
| SET158 | 7.60 |
| SET159 | 9.72 |
| SET171 | 21.81 |
| SET172 | 13.3 |

Regorafenib and sorafenib showed a dose-dependent decrease in cell viability. Intriguingly, treatment with JNK-IN-5A and the target compounds demonstrated a more non-dose-dependent, linear reduction in cell viability at concentrations ranging 30 μM to 30 nM. This suggests that JNK-IN-5A and the target compounds may operate through a different mechanism of action compared to regorafenib and sorafenib, providing a new promising pathway for treatment of HCC.

The expression of proteins associated with the apoptosis pathway and the JNKs target pathway was examined via Western blot analysis. This was conducted on HepG2 cells treated for 2 days with 10 μM of the compounds (FIG. 63). Treatment with both sorafenib and regorafenib elicited a 10 and 16 times higher increase in cleaved PARP, respectively, than the untreated control. Yet, cells treated with SET135 and SET171 exhibited an even higher increase in cleaved PARP (78.31 and 87.97 times, respectively). Additionally, active caspase 3 levels were significantly elevated in cells treated with SET135 (11.85 times) and SET171 (14.66 times), surpassing the increases induced by treatment with sorafenib (4.35 times) and regorafenib (3.61 times). The anti-apoptotic protein Bcl-2, which can inhibit the release of mitochondrial cytochrome C [31], was reduced following treatment with all tested compounds.

The effect of treatment with JNK-IN-5A and the target compounds on their major targets, JNK1/2/3, was evaluated and differences between the JNKs P54 (54 kDa) and p46 (46 kDa) isoforms were observed (FIG. 63). Treatment with all tested compounds resulted in a relatively similar decrease in JNK1/2/3 P54 (0.6 to 0.8 times). The expression levels of JNK1/2/3 p46 were reduced by treatment with sorafenib (0.81 times), regorafenib (0.65 times), and JNK-IN-5A (0.88 times), but not significantly altered by treatment with SET156, SET158, SET159, and SET172, which exhibited less toxicity. SET135 and SET171, however, did reduce JNK1/2/3 p46 expression (0.77 and 0.87 times, respectively). STAT1, which has been reported to regulate PKL expression in liver cells through the JNKs pathway [28], was significantly reduced in all the treated groups (FIG. 63). PKL expression notably decreased in cells treated with JNK-IN-5A and the target compounds. Interestingly, levels of another pyruvate kinase isoform, PKM, did not decrease in comparison to PKL. These findings suggest a specific inhibitory effect on PKL by the target compounds and JNK-IN-5A, which may contribute to their mechanism of action against HCC.

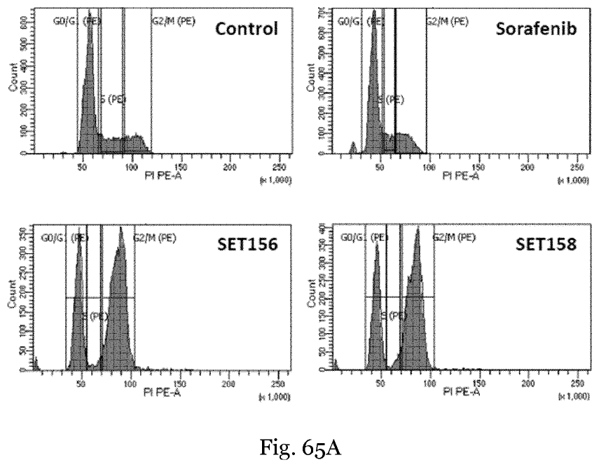

JNK-IN-5A has been previously documented to induce cell cycle arrest and apoptosis through the p53 and p21 pathways in pancreatic cancer and T-cell leukemia [32, 33]. Additionally, its downstream protein, c-Jun, has been identified as an antagonist of p53 in liver cancer [20]. In light of this, the expression level of nuclear P53 in treated HepG2 cells were analysed using Western blot analysis. HepG2 cells treated with 10 μM of SET135 and SET171 for 2 days exhibited a significant increase in nuclear-localized p53 compared to those treated with sorafenib and regorafenib (FIG. 64). Furthermore, the expression of the proto-oncogene c-Myc [34], which is transcriptionally repressed by p53, was reduced following treatment with the tested compounds. Sorafenib and regorafenib decreased nuclear c-Myc expression, consistent with previous studies [35, 36]. Treatment with JNK-IN-5A and the target compounds also significantly reduced c-Myc levels, wherein treatment with SET171 demonstrated the most substantial decrease in comparison to sorafenib and regorafenib. Interestingly, the p53-dependent tumour suppressor gene p21 was found to be localized in the nucleus specifically in the cells treated with JNK-IN-5A and the target compounds, but not in the groups treated with sorafenib or regorafenib. This indicates that JNK-IN-5A and its derivatives may uniquely influence the nuclear translocation of p21 [23], suggesting a distinct mechanism of action that differentiates them from other therapies with sorafenib and regorafenib.

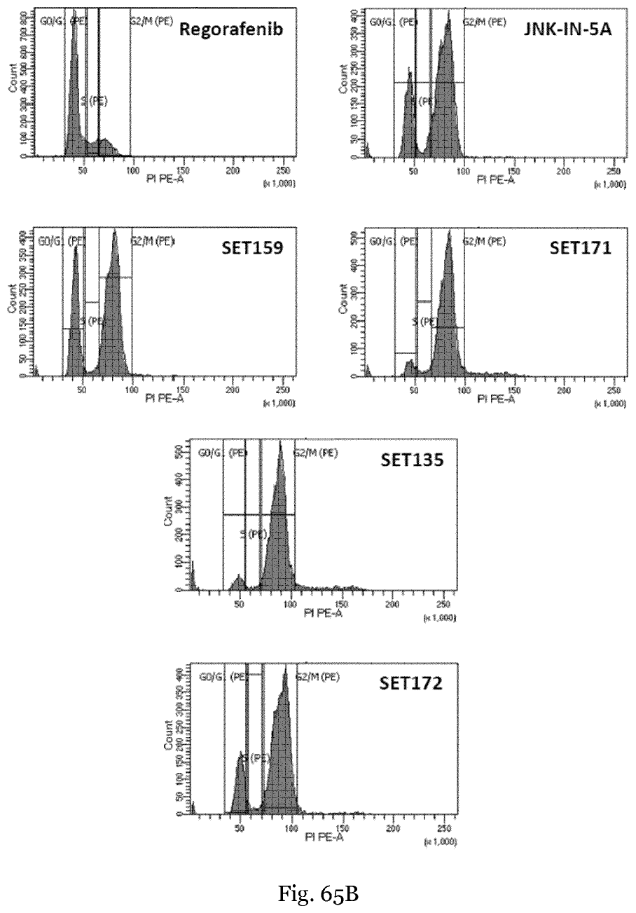

The p53 tumour suppressor protein is extensively recognized for its role in inducing apoptosis, cell cycle arrest, and autophagy [37-40] Studies suggest that both P53 and p21 are crucial for maintaining cell viability, with IC50 assays indicating that treatment with JNK-IN-5A and target compounds exert a robust cell cycle arrest effect. To verify this, a propidium iodide (PI) fluorescence-activated cell sorting (FACS) analysis was conducted (FIG. 65A-B). PI staining and FACS analysis confirmed significant G2/M cell cycle arrest in cells treated with JNK-IN-5A and target compounds (FIG. 65A-B and Table 6).

TABLE 6
Cell population on PI FACs analysis. JNK-IN-5A and target
compounds induce G2/M cell cycle arrest. SET135 and SET171
showed much stronger effect than other compounds.

| | G0/G1 | S | G2/M |
|---|---|---|---|
| Control | 62.4 | 15.4 | 16.2 |
| Sorafenib | 62.2 | 10.1 | 19.5 |
| Regorafenib | 70.5 | 9.7 | 14.9 |
| JNK-IN-5A | 22.7 | 4.4 | 67.3 |
| SET135 | 5.5 | 2.0 | 79.9 |
| SET156 | 30.1 | 2.3 | 60.8 |
| SET158 | 31.6 | 3.5 | 60.3 |
| SET159 | 30.5 | 3.3 | 62.4 |
| SET171 | 6.6 | 3.6 | 79.1 |
| SET172 | 16.7 | 2.7 | 73.2 |

In contrast, sorafenib and regorafenib did not alter the cell cycle distribution significantly compared to the control group. Histogram plots revealed that JNK-IN-5A, SET156, SET158, SET159, and SET172 maintained 20 to 30% of the cells in the G0/G1 phase and 60 to 70% in the G2/M phase. SET135 and SET171 induced a more pronounced cell cycle arrest, with approximately 5 to 6% of cells in G0/G1 and around 89% in the G2/M phase.

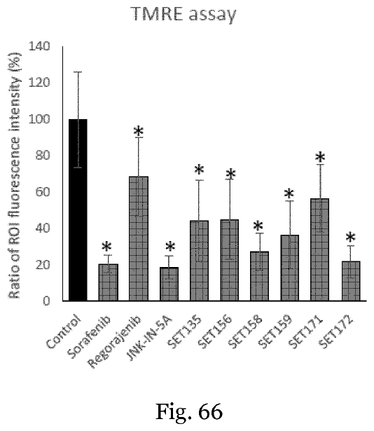

Bcl-2, a protein downstream of JNKs involved in the apoptotic pathway [41], has been reported to facilitate mitochondrial apoptosis when phosphorylated [32]. To assess mitochondrial function during apoptosis, the mitochondrial membrane potential was examined using TMRE staining (FIG. 66). HepG2 cells treated with 10 μM of sorafenib and regorafenib for one day showed a decrease in mitochondrial membrane potential, as previously reported [42]. Similarly, treatment with JNK-IN-5A, SET135, SET156, SET158, SET159, SET171, and SET172 also significantly decreased mitochondrial membrane potential.

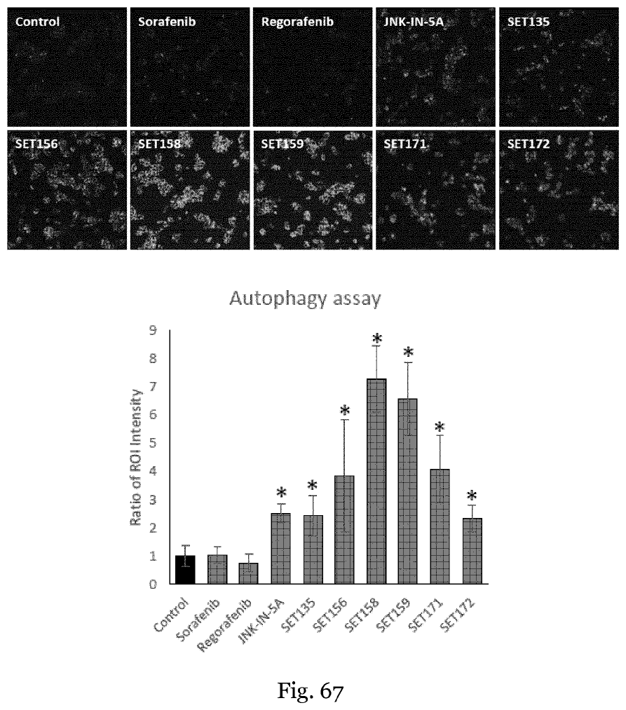

Furthermore, JNK and Bcl-2 are known to participate in autophagic cell death [43,44]. Autophagy assays were, thus, performed on HepG2 cells treated with 10 μM of the compounds for one day (FIG. 67). Notably, while treatment with sorafenib and regorafenib did not induce autophagy, JNK-IN-5A and target compounds significantly increased autophagy GFP signals. These findings collectively suggest that JNK-IN-5A and its derivatives prompt apoptotic cell death through a multifaceted mechanism involving strong cell cycle arrest, diminished mitochondrial membrane potential, and pronounced autophagy, all mediated via the p53 pathway. The implications of these effects underline the potential of the target compounds as multifunctional agents in cancer therapy, particularly in targeting HCC.

Thus, it is shown herein that treatment with JNK-IN-5A and target compounds induce apoptotic cell death, cell cycle arrest, and autophagy through the activation of the p53 tumour suppressor pathway. This mechanism highlights the compounds' ability to engage a critical regulatory pathway in cell biology, leveraging the p53 protein's role in maintaining cellular integrity by prompting responses to DNA damage, stress signals, and aberrant growth signals. By activating this pathway, JNK-IN-5A and the target compounds effectively promote the removal of damaged or cancerous cells, representing a targeted approach to cancer therapy that harnesses the body's natural defence mechanisms against tumour progression.

SET135 and SET171 have Specific Necrotic Cell Death Program.

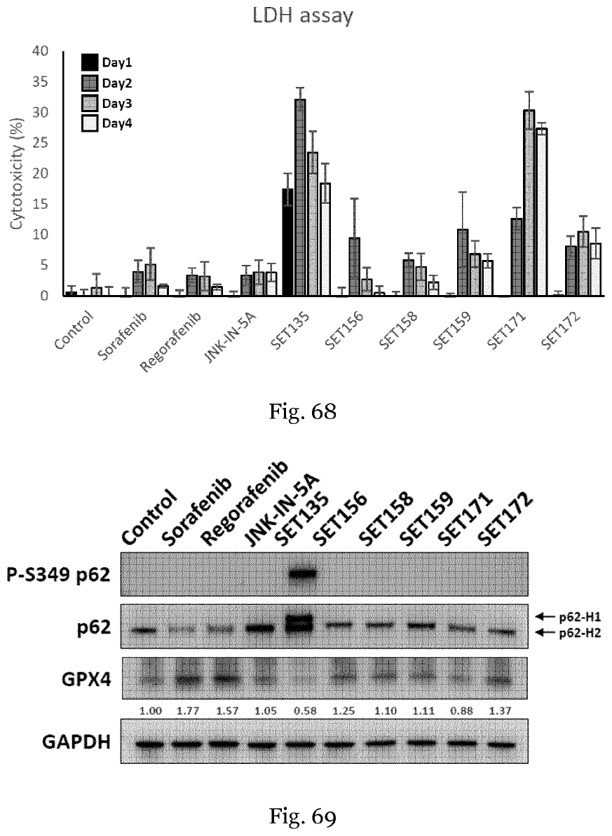

The progression of necrotic cell death was monitored over a period of up to 4 days. Notably, treatment with 10 μM of SET135 induced a pronounced necrotic cell death from day 1 to day 4 (FIG. 68). As previously mentioned, SET135 also triggers apoptosis (FIG. 63). The concurrent induction of apoptosis and necrosis that is observed is reminiscent of the effects reported for Tanshinone IIA treatment of HepG2 cells, a process termed necroptosis [45]. Necroptosis is characterized as programmed cell necrosis, distinguishing it from apoptosis in several key ways [46, 47]. Unlike apoptosis, during necroptosis, the protein p62/SQSTM1 recruits autophagic proteins to form a necrosome, leading to necrotic cell death [48]. To further understand this process in the context of SET135 treatment, the expression of p62 (SQSTM1) and its phosphorylated form, phospho-p62 (P-S349), an activated state of p62, was analysed using Western blot analysis (FIG. 69). p62 and its phosphorylated form at serine 349 (p-S349 p62), is recognized as a potent oncogenic protein [49]. The phosphorylation of p62 at serine 349 enhances its binding affinity to the Keap1 protein, leading to the activation of the Nrf2 pathway. This pathway is well-known for its cytoprotective functions in normal cells. However, it also plays a role in tumorigenesis in pre-malignant cells, contributing to drug resistance and facilitating tumour growth. p62, which is a crucial component of protein aggregates forming Mallory-Denk bodies (MDBs) or intracellular hyaline bodies (IHBs), is considered both a prognostic and diagnostic marker for NASH and HCC58-60. A recent study has differentiated two isoforms of p62: p62-H1, the full-length isoform, and p62-H2, which is partially devoid of the PB1 domain [50]. These isoforms differ in their aggregation potential. Notably, p62-H2 is more predominantly expressed in human liver tissue than p62-H1.

Treatment of HepG2 cells with 10 μM SET135 for 2 days resulted in significantly elevated levels of both p62 and P-S349 p62. Interestingly, the SET135 treated group exhibited not only an increase in p62-H2 but also a significant upregulation of p62-H1. This observation suggests that SET135 may induce necroptosis in HepG2 cells through mechanisms involving both the upregulation of p62 and its phosphorylated form, contributing to the formation of the necrosome complex. The dual action of SET135, promoting both apoptosis and necroptosis, underlines its potential as a multifaceted therapeutic agent, offering insights into its mechanism of action.

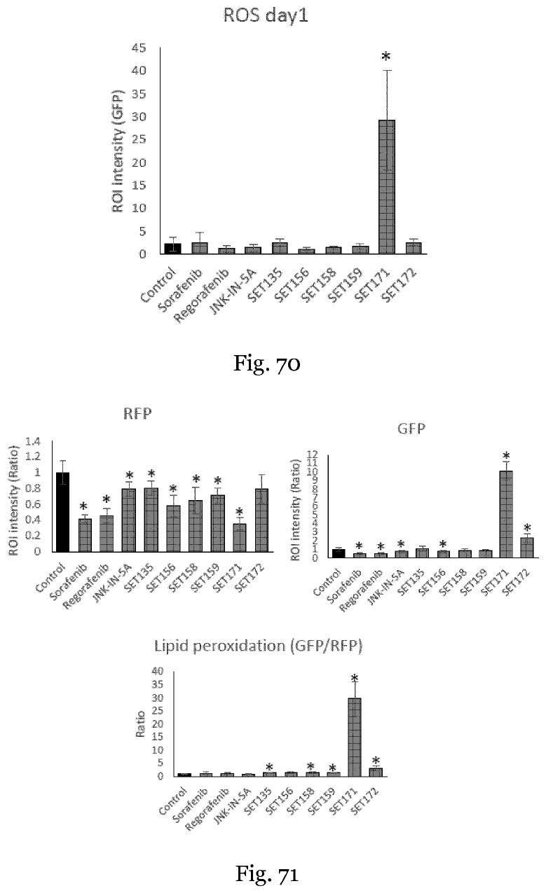

Oxidative stress can induce cell death through multiple pathways, including mitochondrial damage via caspase-3 activation, DNA damage through cleaved PARP, and the initiation of autophagy [51-54]. To understand the mechanism behind cell death induced by JNK-IN-5A and the target compounds, the cellular reactive oxygen species (ROS) levels in HepG2 cells treated with 10 μM of the compounds for one day were assessed. Cells were stained with the ROS detector DCFDA, and fluorescence image analysis showed that treatment with SET171 significantly increased cellular ROS (FIG. 70). Notably, SET171 was associated with pronounced late necrotic cell death from day 3 to 4 (FIG. 68).

Previous studies have demonstrated that elevated cellular ROS can induce lipid peroxidation, leading to membrane rupture and necrotic cell death, a process referred to as ferroptosis [40,41]. To confirm lipid peroxidation, we employed a ratiometric lipid peroxidation sensor staining (FIG. 71). This sensor stains cellular lipids with RFP fluorescence, which is replaced by GFP fluorescence upon lipid peroxidation. Treatment with SET171 exhibited a statistically significant increase in the GFP/RFP ratio, indicating lipid peroxidation, unlike other compounds tested. The expression level of glutathione peroxidase 4 (GPX4), a crucial enzyme that mitigates lipid peroxidation and plays a key role in preventing ferroptosis [55], was also measured. HepG2 cells treated with sorafenib and regorafenib showed significantly higher GPX4 expression compared to the control (FIG. 69), while treatments with JNK-IN-5A, SET156, SET158, SET159, and SET172 resulted in similar or slightly increased GPX4 levels (1.05 to 1.37 times). In contrast, treatment with SET135 and SET171 showed a marked decrease in GPX4 expression levels (0.58 times and 0.88 times, respectively).

These findings suggest that SET171 induces ferroptosis through mechanisms involving elevated cellular ROS, lipid peroxidation, and reduced GPX4 expression. The ability of SET135 and SET171 to simultaneously induce apoptosis and necrosis, including ferroptosis, presents a promising approach to overcoming resistance to apoptosis in cancer cells, highlighting their potential as therapeutic agents in cancer treatment strategies [56].

### Target Compounds Inhibit Cancer Invasion and Mobility in HepG2

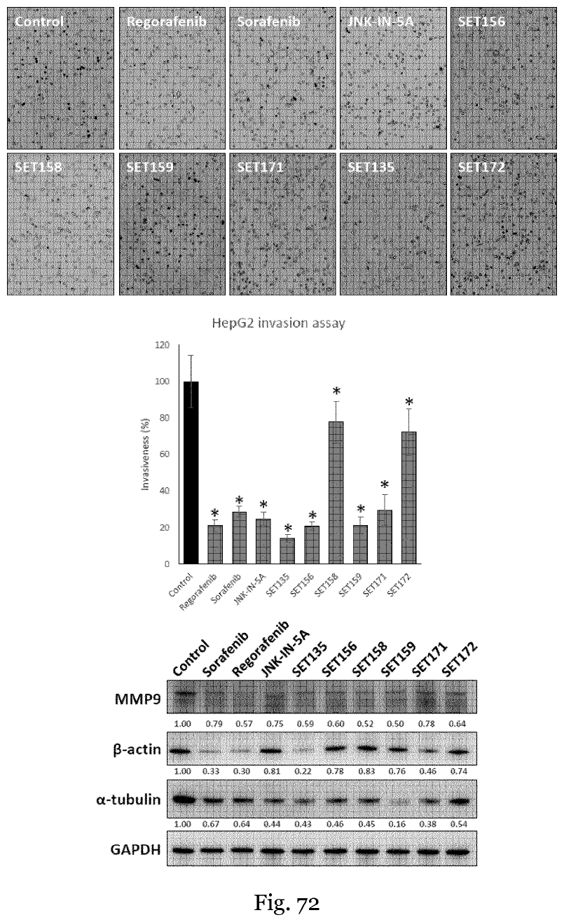

The impact of JNK-IN-5A, target compounds, sorafenib and regorafenib, on the invasiveness and motility of HepG2 cancer cells was assessed. Western blot analysis indicated that treatment with all the tested compounds led to a decrease in MMP9 levels (FIG. 72), a matrix metalloproteinase implicated in cancer invasion and metastasis. Furthermore, the expression levels of proteins involved in cancer cell migration and motility, such as α-tubulin [57-59] and β-actin [60, 61], were significantly reduced. An invasion assay conducted with a concentration of 1 μM revealed that treatment with regorafenib, sorafenib, JNK-IN-5A, and the target compounds substantially reduced the invasiveness of HepG2 cells. Specifically, treatment with regorafenib reduced invasiveness by 21.0%±3.1, sorafenib by 28.7%±2.9, JNK-IN-5A by 24.9%±3.8, SET156 by 14.0%±1.9, SET158 by 20.8%±2.4, SET159 by 77.8%±11.5, SET171 by 21.4%±4.3, SET135 by 29.5%±8.3, and SET172 by 72.3%±12.6 (FIG. 72).

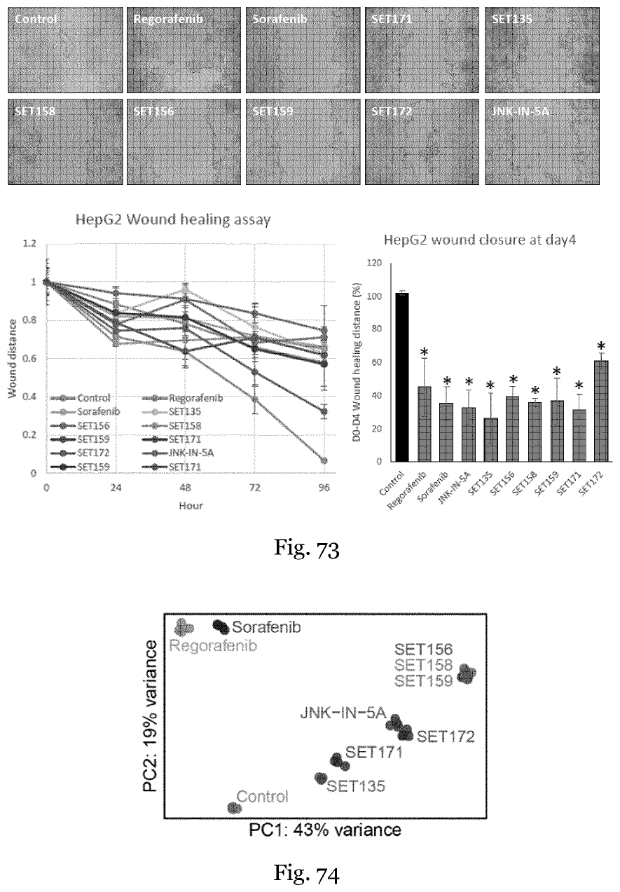

Additionally, a wound healing assay was performed to evaluate the compounds' effect on cancer cell motility. After achieving a confluent state, a wound was created in the HepG2 cell layer using a pipette tip, and the wound width was measured through image analysis until the control HepG2 cells closed the wounded area by day 4 (FIG. 73). The results showed that all tested compounds delayed wound healing compared to untreated HepG2 cells, which achieved 100% wound closure by day 4. Specifically, treatment with regorafenib delayed wound healing by 44.8%±17.4, sorafenib by 35.0%±9.9, JNK-IN-5A by 32.7%±10.4, SET156 by 26.2%±15.3, SET158 by 39.5%±6.1, SET159 by 35.5%±2.6, SET171 by 36.4%±14.0, SET135 by 31.2%±9.4, and SET172 by 60.7%±5.0. These findings suggest that the tested compounds effectively impair the invasiveness and motility of HepG2 cells, highlighting their potential as therapeutic agents in limiting cancer progression and metastasis.

### Target Compounds Show Distinct Transcriptional Profiles to Regorafenib and Sorafenib in HepG2 Cells

To elucidate the changes in gene expression and biological pathways induced by drug treatment, RNA sequencing (RNA-seq) analysis was conducted on HepG2 cells treated with JNK-IN-5A, target compounds, regorafenib and sorafenib. The analysis revealed distinct transcriptional profiles for each treatment group compared to the control (FIG. 74). Notably, the transcriptional responses to regorafenib and sorafenib were more similar to each other than to the responses elicited by JNK-IN-5A and the target compounds. This suggests that JNK-IN-5A and the target compounds may activate different mechanistic pathways from those targeted by regorafenib and sorafenib. Among the target compounds, SET172 displayed the closest transcriptional similarity to JNK-IN-5A. SET156, SET158, and SET159 clustered closely together, indicating similar transcriptional changes, while SET135 and SET171 constituted another distinct group (FIG. 74).

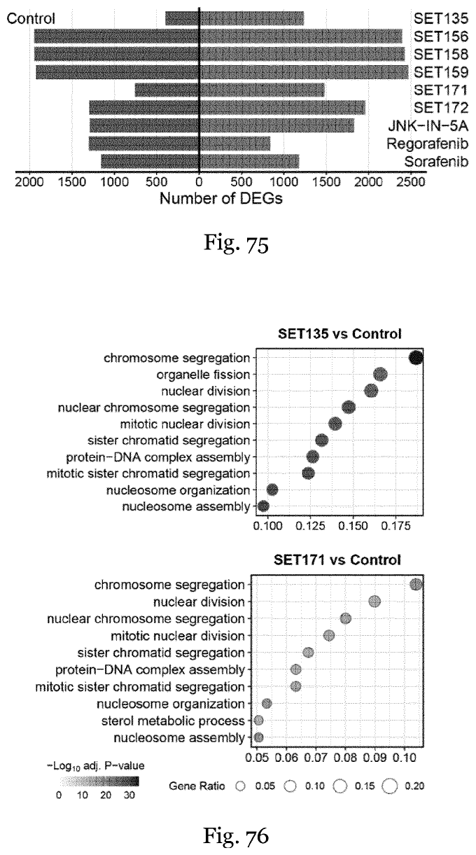

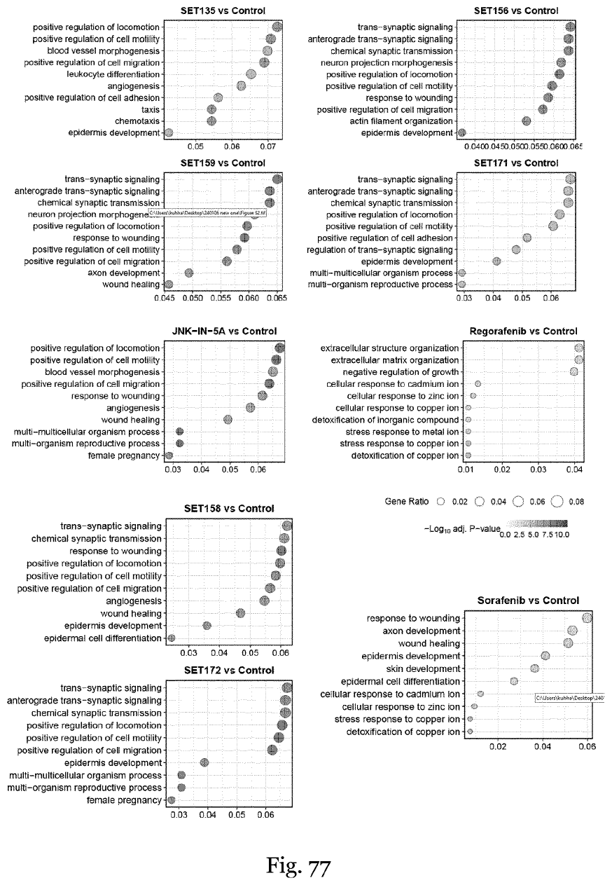

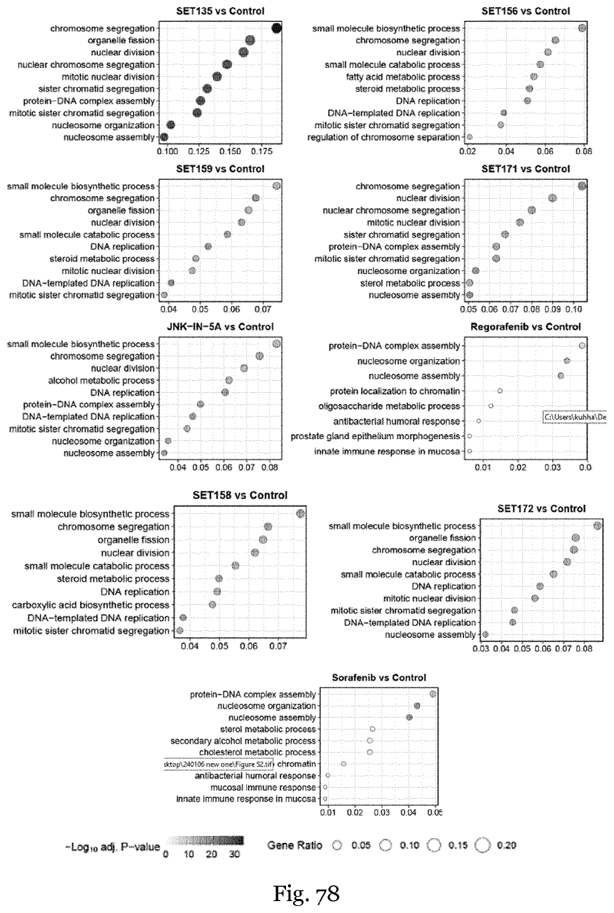

In terms of differentially expressed genes (DEGs), regorafenib and sorafenib induced a smaller number of DEGs compared to the other treatments. In contrast, SET135 and SET171 exhibited a moderate number of DEGs, highlighting their potential for inducing significant transcriptional changes (FIG. 75). Gene ontology (GO) analysis and gene set overrepresentation analysis (GSOA) of these DEGs revealed a significant association with cell cycle regulation. Specifically, DEGs downregulated by treatment with SET135 and SET171 were closely linked to cell cycle processes, with SET135 showing a more pronounced effect than SET171 and other target compounds (FIG. 76-78). This RNA-seq analysis underlines the unique impacts of JNK-IN-5A and target compounds on HepG2 cells, particularly in modulating cell cycle-related genes, and distinguishes their effects from those of regorafenib and sorafenib, suggesting a novel mechanism of action for these compounds in cancer treatment.

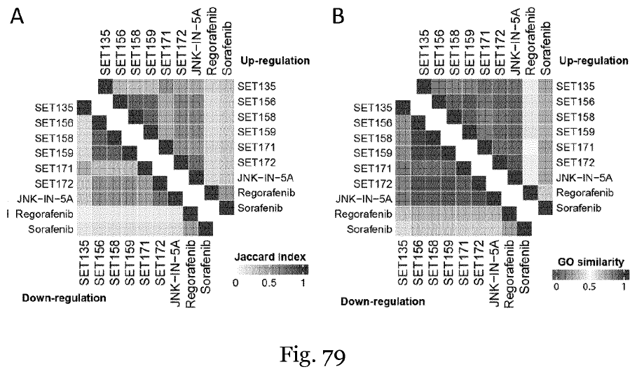

Hierarchical clustering analysis of gene expression profiles revealed that the compounds could be categorized into three distinct clusters beyond the control group. These clusters include regorafenib and sorafenib as one group, SET135 and SET171 as another, and the last cluster comprising the remaining target compounds, and JNK-IN-5A. To further assess the similarity between differentially expressed genes (DEGs) resulting from various treatments, the Jaccard index and semantic similarity analyses [62] based on significant gene ontology (GO) biological pathways associated with these DEGs was analysed. The outcomes of these analytical approaches were congruent, demonstrating that regorafenib and sorafenib exhibited less similarity to JNK-IN-5A and the target compounds (FIG. 79). This finding aligns with the results obtained from principal component analysis (PCA) and clustering analysis (FIGS. 74 and 79A), reinforcing the notion that JNK-IN-5A and the target compounds potentially engage distinct biological mechanisms compared to regorafenib and sorafenib. Within the group of JNK-IN-5A and target compounds, SET156, SET158, and SET159 were closely related, indicating a high degree of similarity among their effects. Treatment with these compounds also showed a notable resemblance to treatment with JNK-IN-5A, suggesting shared pathways or targets that might underlie their therapeutic actions.

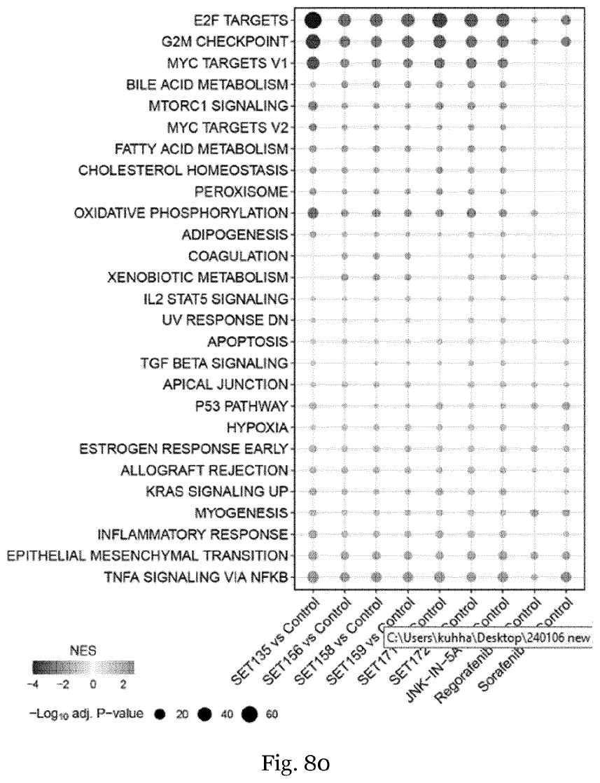

To thoroughly assess the pathways disrupted by the treatments, gene set enrichment analysis (GSEA) was conducted utilizing hallmark gene sets from the Molecular Signatures Database (MSigDB) [63] and the Kyoto Encyclopaedia of Genes and Genomes (KEGG) pathways [64]. All target compounds inhibited the cell cycle-related pathways, such as E2F targets and the G2M checkpoint, more effectively than regorafenib and sorafenib (FIG. 80). Among these, SET135 was identified as the most potent inhibitor, closely followed by SET171. Moreover, all treatments were found to upregulate apoptosis signalling. Distinctly, treatment with JNK-IN-5A and its derivatives exhibited liver-specific effects, notably inhibiting bile acid metabolism and fatty acid metabolism pathways. Additionally, oxidative phosphorylation (OXPHOS) was uniquely suppressed by treatments involving JNK-IN-5A and the target compounds These findings underline that while all treatments robustly hinder the cell cycle, JNK-IN-5A and the target compounds offer additional liver-specific actions that contribute to their efficacy in inhibiting cell growth. This suggests a broader spectrum of therapeutic effects, particularly relevant for liver-related conditions, underlining the potential of these compounds in targeted cancer therapy, especially in HCC.

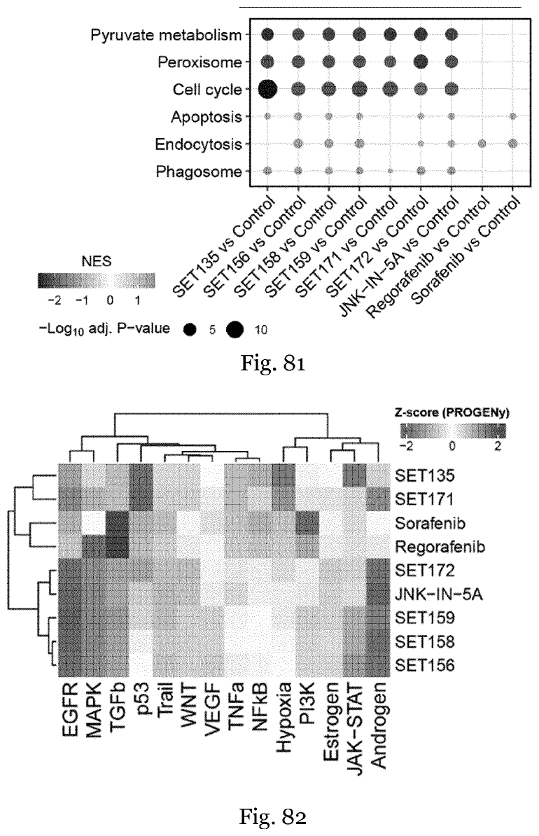

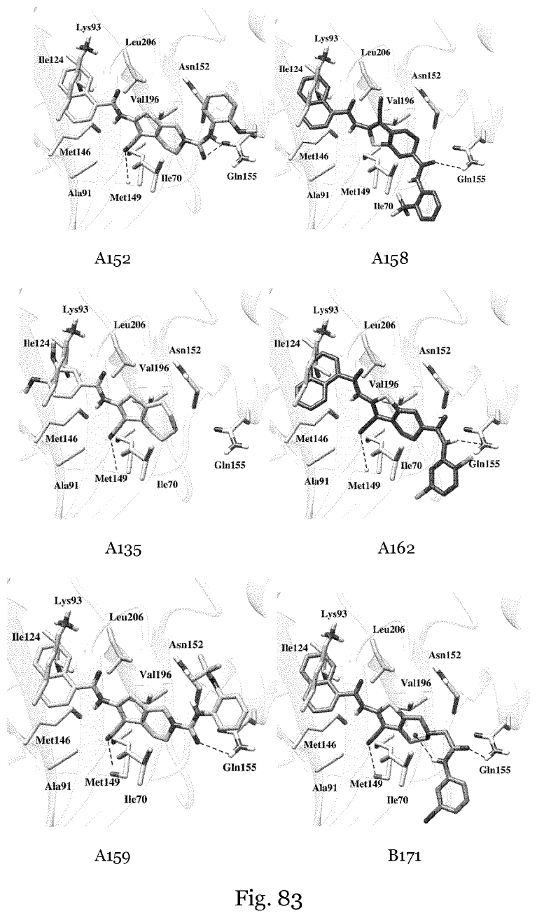

The findings derived from the analysis of hallmark gene sets were further corroborated by KEGG pathway analysis. Pyruvate metabolism was significantly suppressed in all treatments involving JNK-IN-5A and target derivatives (FIG. 81-82). Moreover, a pronounced inhibition of the cell cycle was observed with these treatments, notably with SET135 displaying the most substantial effect. Additionally, there was significant suppression of the peroxisome pathway, which may relate to autophagy, in treatments with JNK-IN-5A and target compounds, unlike with regorafenib and sorafenib. When evaluating pathways associated with programmed cell death, such as apoptosis, endocytosis, and phagosome, the analysis did not reveal a clear distinction between the effects of treatment with target compounds and treatment with sorafenib and regorafenib. This indicates that while treatment with JNK-IN-5A and target compounds uniquely impact certain metabolic and cell cycle pathways, their influence on programmed cell death pathways might share similarities with that of regorafenib and sorafenib. These insights suggest that the unique therapeutic potential of JNK-IN-5A and target compounds could be attributed to their specific actions on metabolic regulation and cell cycle control, rather than a fundamentally different approach to inducing programmed cell death.

Utilizing PROGENy, a computational algorithm designed to infer the activity of 14 cancer-related pathways from gene expression data [65], it was discovered that SET135 and SET171 significantly activated the p53 pathways, corroborating the findings illustrated in FIG. 66. Furthermore, while treatment with regorafenib and sorafenib exhibited a high level of TGFβ signalling pathway activation, such activation was not observed in treatments involving JNK-IN-5A and the target compounds. This points to a distinct functional profile of the target compounds compared to the sorafenib and regorafenib.

The comprehensive analyses in this example utilizing HepG2 cells indicate that although all tested treatments are capable of inhibiting cancer cell growth—with SET135 and SET171 treatment being the most effective—the target compounds and JNK-IN-5A likely possess unique and liver-specific effects that distinguish them from regorafenib and sorafenib. This distinction suggests that the target compounds, especially SET135 and SET171, might offer alternative therapeutic mechanisms, potentially providing new avenues for the treatment of liver-related cancers, particularly by targeting pathways such as p53 which are crucial in the regulation of cancer cell survival and death.

### Example A

An in vivo toxicological study was performed to assess the potential toxicity, in terms of clinical signs, body weight and clinical pathological parameters, of PKL-A135, PKL-A152, PKL-A159 and PKL-B171 in rats when administered orally. In this example (like Example 2) the derivatives will be denoted as SET135 (PKL-A135), SET152 (PKL-A152), SET159 (PKL-A159), and SET171 (PKL-B171).

### Methods

### Formulations

The target compounds were formulated prior to administration (Table 7).

TABLE 7
The formulations used in the toxicological study.

| Compound | Vehicle | Physical form |
|---|---|---|
| Control | 1.5 w/w % HPMC, 1.5 | n.a. |
| | w/w % PS80 | |
| | in 10 mM PBS, pH 7 | |
| SET135 | 1.5 w/w % HPMC, 1.5 | Liquid suspension, |
| | w/w % PS80 | milky white colour |
| | in 10 mM PBS pH 7. | |
| SET152 | 2 w/w % HPMC, 2 | Liquid suspension, |
| | w/w % PS80 in | yellow colour with |
| | 10 mM PBS pH 7. | black precipitations |
| SET159 | 20 w/w % Gelucire 48/16 | Liquid suspension, |
| | | milky white colour |
| SET171 | 20 w/w % Gelucire 48/16 | Liquid suspension, |
| | | milky white colour |

### Animals and Dosing

Forty-two male and forty-two female rats of the Wistar strain were allocated to the study. Three groups of three males and three females were given doses of 30, 100 and 300 mg/kg of target compounds (Table 8). The vehicle was 1.5 w/w % HPMC, 1.5 w/w % PS80 in 10 mM PBS, pH 7 and dose volume were 5 mL/kg.

TABLE 8
Study design with details on animals and dosing.

| | | Number | Prelim. | Dose | Dose |
|---|---|---|---|---|---|
| Gr | | of | Dose | conc. | vol |
| No | Compound | Animals | mg*/kg | mg/mL | mL/kg |
| 1 | Fel! Hittar | 4 M + 4 F | 0 | 0 | 5 |
| | inte | | | | |
| | referenskä | | | | |
| | lla. | | | | |
| 2 | SET-135Fel! | 3 M + 3 F | 30 | 6 | 5 |
| | [symbol] | | | | |
| 3 | SET-135 | 3 M + 3 F | 100 | 20 | 5 |
| 4 | SET-135 | 3 M + 3 F | 300 | 60 | 5 |
| 5 | SET-152 | 3 M + 3 F | 30 | 6 | 5 |
| 6 | SET-152 | 3 M + 3 F | 100 | 20 | 5 |
| 7 | SET-152 | 3 M + 3 F | 300 | 60 | 5 |
| 8 | SET-159 | 3 M + 3 F | 30 | 6 | 5 |
| 9 | SET-159 | 3 M + 3 F | 100 | 20 | 5 |
| 10 | SET-159 | 3 M + 3 F | 300 | 60 | 5 |
| 11 | SET-171 | 3 M + 3 F | 30 | 6 | 5 |
| 12 | SET-171 | 3 M + 3 F | 100 | 20 | 5 |
| 13 | SET-171 | 3 M + 3 F | 300 | 60 | 5 |

[symbol]

indicates data missing or illegible when filed

The animals were acclimatised at least 14 day prior to the study and exposed to a 12 hours light, 12 hours dark light cycle. The formulations were administered via oral gavage. Administration occurred once daily in the morning for seven consecutive days.

### Clinical Observations and Measurements

A check of animal health and welfare status was done at least once on working days and weekend days.

Animals were observed for extended clinical observations on Day 1 and at least once at the following intervals: 0-1-hour; 1-3-hours; 3-6 hours; 6-24 hours after dosing at each dose level.

Body weights were recorded the week before and within 24 hours before start of dosing, on Day 3 and on necropsy Day 8.

Water consumption was not recorded but checked by visual inspections.

All animals were bled for bioanalysis (lipid and metabolite analysis) from the tail vein at 2 hours after dosing on Day 1 and Day 7.

Blood samples (at least 60 μL blood in anticoagulant (K₂EDTA)) were placed on ice after sample collection and centrifuged at ~3000×G for 5 min at 4° C. All plasma were transferred into a separate low binding protein tube. The tubes were frozen upright at approximately −20° C. The whole sample processing was made within 30 minutes after sampling.

### Clinical Pathology

Blood for scheduled haematology and plasma chemistry were taken at necropsy. For plasma chemistry, 0.6 ml blood (lithium heparin) per animal and for haematology, 0.5 ml blood (K₂EDTA) per animal. The blood and plasma were analysed using Exigo analyzer and Vetscan equipment.

### Haematology Sample Procedures

Blood was analysed within 60 min of sampling. On the morning of analysis, the Exigo analyzer was checked against a reference sample provided by the manufacturer, and a control sample analysis cycle was performed (Table 9).

TABLE 9
The haematology parameters analysed and their abbreviations.

| | |
|---|---|
| Red Blood cells | RBC |
| Red cell distribution width (%) | RDW % |
| Red cell distribution width (absolute) | RDWa |
| Mean Red cell hemoglobin | MCH |
| Hematocrit | HCT |
| Mean Red cell hemoglobin concentration | MCHC |
| Mean Red cell volume | MCV |
| Hemoglobin | HGB |
| Platelets | PLT |
| Mean Platelet volume | MPV |
| White blood cells | WBC |
| Lymphocytes | LYM |
| Granulocytes | GRAN |
| Monocytes | MONO |

### Plasma Chemistry Sample Procedures

Blood samples were kept in room temperature (−20° C.) after collection and mixing. The blood samples were analysed as whole blood within 60 min of collection. The VetScan analyser was calibrated with calibration samples of known plasma levels of each parameter before start of analysis, occasionally during the analysis period of study samples and after completed analyses (Table 10).

TABLE 10
The plasma chemistry parameters analysed and their abbreviations.

| | |
|---|---|
| Albumin | ALB |
| Alanine aminotransferase | ALT |
| Alkaline phosphatase | ALP |
| Amylase | AMYL |
| Bilirubin (total) | TBIL |
| Blood urea nitrogen | BUN |
| Calcium | Ca²⁺ |
| Creatinine | CREA |
| Globulin | GLOB |
| Glucose | GLU |
| Phosphate | PHOS |
| Potassium | K⁺ |
| Sodium | Na⁺ |
| Total Protein | TP |

### Pathology

Animals were killed for scheduled necropsy on Study Day 8 by exsanguination from a common carotid artery under isoflurane and O₂ anaesthesia.

The body weight was recorded in all animals at scheduled necropsy after exsanguination. For all necropsies, external features were inspected, the cranial, thoracic and abdominal cavities and contents were examined. Macroscopic abnormalities were recorded.

Intact and unfixed organ weights of kidney and liver were determined.

### Results

### Clinical Observations

24 observations were performed on all the tested groups, respectively, between day 1 and day 8 of the study. No clinically adverse effects from the test compounds were seen in any of the treated animals, at any dosage (30-300 mg/kg).

Furthermore, there were no effects on the body weight related to the treatment in this study.

### Clinical Pathology

As compared to the corresponding control group (Group 1), no alterations in haematology variables were noted in males nor females of the groups treated with any of the target compounds, at any dose levels (Tables 11-14).

TABLE 11
The Haematology parameters of the control group 1
and the groups treated with 30-300 mg/kg of SET135.

| | Sex/Group | | | | | | | |
|---|---|---|---|---|---|---|---|---|
| | M/1 | M/2 | M/3 | M/4 | F/1 | F/2 | F/3 | F/4 |
| WBC | 6.4 | 8.6 | 7.4 | 7.5 | 6.3 | 6.6 | 8.3 | 6.4 |
| (10⁹/l) | | | | | | | | |
| LYM | 5.2 | 6.5 | 6.1 | 6.2 | 5.3 | 5.4 | 6.7 | 5.1 |
| (10⁹/l) | | | | | | | | |
| MONO | 0.13 | 0.30 | 0.17 | 0.20 | 0.15 | 0.17 | 0.27 | 0.20 |
| (10⁹/l) | | | | | | | | |
| GRAN | 1.10 | 1.83 | 1.17 | 1.13 | 0.85 | 0.97 | 1.33 | 1.10 |
| (10⁹/l) | | | | | | | | |
| HGB | 14.9 | 15.0 | 14.6 | 14.9 | 14.2 | 14.2 | 14.4 | 14.7 |
| (g/l) | | | | | | | | |
| MCH | 19.8 | 19.7 | 19.6 | 20.7 | 19.4 | 19.5 | 19.9 | 20.8 |
| (pg) | | | | | | | | |
| MCHC | 34.6 | 33.9 | 33.7 | 34.0 | 34.9 | 34.1 | 34.1 | 34.2 |
| (g/l) | | | | | | | | |
| RBC | 7.58 | 7.62 | 7.43 | 7.20 | 7.31 | 7.26 | 7.21 | 7.04 |
| (10¹²/l) | | | | | | | | |
| MCV | 57.1 | 58.2 | 58.1 | 60.9 | 55.7 | 57.4 | 58.4 | 60.8 |
| (fl) | | | | | | | | |
| HCT | 43.2 | 44.3 | 43.2 | 43.8 | 40.6 | 41.7 | 42.1 | 42.8 |
| (%) | | | | | | | | |
| RDW % | 14.4 | 14.1 | 14.4 | 14.0 | 14.1 | 14.2 | 13.7 | 13.8 |
| (%) | | | | | | | | |
| PLT | 806 | 841 | 815 | 924 | 1005 | 1014 | 978 | 1011 |
| (10⁹/l) | | | | | | | | |

TABLE 12
The Haematology parameters of the control group 1
and the groups treated with 30-300 mg/kg of SET152.

| | Sex/Group | | | | | | | |
|---|---|---|---|---|---|---|---|---|
| | M/1 | M/5 | M/6 | M/7 | F/1 | F/5 | F/6 | F/7 |
| WBC | 6.4 | 7.7 | 8.5 | 7.2 | 6.3 | 6.8 | 7.1 | 6.0 |
| (10⁹/l) | | | | | | | | |
| LYM | 5.2 | 5.9 | 6.9 | 5.8 | 5.3 | 5.6 | 5.7 | 4.8 |
| (10⁹/l) | | | | | | | | |
| MONO | 0.13 | 0.23 | 0.17 | 0.17 | 0.15 | 0.17 | 0.17 | 0.23 |
| (10⁹/l) | | | | | | | | |
| GRAN | 1.10 | 1.50 | 1.47 | 1.17 | 0.85 | 1.00 | 1.20 | 1.03 |
| (10⁹/l) | | | | | | | | |
| HGB | 14.9 | 15.6 | 15.1 | 15.4 | 14.2 | 14.1 | 14.3 | 14.7 |
| (g/l) | | | | | | | | |
| MCH | 19.8 | 19.6 | 19.8 | 19.7 | 19.4 | 19.5 | 19.5 | 19.7 |
| (pg) | | | | | | | | |
| MCHC | 34.6 | 33.8 | 33.8 | 33.8 | 34.9 | 34.1 | 34.0 | 34.1 |
| (g/l) | | | | | | | | |
| RBC | 7.58 | 7.97 | 7.63 | 7.80 | 7.31 | 7.24 | 7.32 | 7.43 |
| (10¹²/l) | | | | | | | | |
| MCV | 57.1 | 58.0 | 58.8 | 58.4 | 55.7 | 57.2 | 57.4 | 57.7 |
| (fl) | | | | | | | | |
| HCT | 43.2 | 46.3 | 44.7 | 45.6 | 40.6 | 41.5 | 42.0 | 42.9 |
| (%) | | | | | | | | |
| RDW % | 14.4 | 14.2 | 14.6 | 14.4 | 14.1 | 13.9 | 14.0 | 14.1 |
| (%) | | | | | | | | |
| PLT | 806 | 922 | 990 | 1046 | 1005 | 1089 | 1122 | 1062 |
| (10⁹/l) | | | | | | | | |

TABLE 13
The Haematology parameters of the control group 1
and the groups treated with 30-300 mg/kg of SET159.

| | Sex/Group | | | | | | | |
|---|---|---|---|---|---|---|---|---|
| | M/1 | M/8 | M/9 | M/10 | F/1 | F/8 | F/9 | F/10 |
| WBC | 6.4 | 3.9 | 8.7 | 5.7 | 6.3 | 5.9 | 6.7 | 6.4 |
| (10⁹/l) | | | | | | | | |
| LYM | 5.2 | 2.8 | 7.1 | 4.6 | 5.3 | 4.9 | 5.8 | 5.3 |
| (10⁹/l) | | | | | | | | |
| MONO | 0.13 | 0.10 | 0.23 | 0.10 | 0.15 | 0.13 | 0.10 | 0.17 |
| (10⁹/l) | | | | | | | | |
| GRAN | 1.10 | 1.03 | 1.43 | 1.00 | 0.85 | 0.80 | 0.77 | 0.93 |
| (10⁹/l) | | | | | | | | |
| HGB | 14.9 | 14.5 | 14.9 | 14.8 | 14.2 | 14.4 | 14.5 | 14.5 |
| (g/l) | | | | | | | | |
| MCH | 19.8 | 19.9 | 19.9 | 19.6 | 19.4 | 19.4 | 18.9 | 19.4 |
| (pg) | | | | | | | | |
| MCHC | 34.6 | 35.4 | 35.3 | 35.1 | 34.9 | 35.7 | 35.2 | 35.2 |
| (g/l) | | | | | | | | |
| RBC | 7.58 | 7.26 | 7.51 | 7.59 | 7.31 | 7.39 | 7.66 | 7.45 |
| (10¹²/l) | | | | | | | | |
| MCV | 57.1 | 56.3 | 56.4 | 55.8 | 55.7 | 54.5 | 53.8 | 55.2 |
| (fl) | | | | | | | | |
| HCT | 43.2 | 40.9 | 42.4 | 42.4 | 40.6 | 40.3 | 41.2 | 41.1 |
| (%) | | | | | | | | |
| RDW % | 14.4 | 15.1 | 15.4 | 15.3 | 14.1 | 14.1 | 14.6 | 14.4 |
| (%) | | | | | | | | |
| PLT | 806 | 1117 | 974 | 1200 | 1005 | 1028 | 994 | 808 |
| (10⁹/l) | | | | | | | | |

TABLE 14
The Haematology parameters of the control group 1
and the groups treated with 30-300 mg/kg of SET171.

| | Sex/Group | | | | | | | |
|---|---|---|---|---|---|---|---|---|
| | M/1 | M/11 | M/12 | M/13 | F/1 | F/11 | F/12 | F/13 |
| WBC | 6.4 | 7.7 | 8.1 | 8.6 | 6.3 | 5.5 | 5.2 | 7.1 |
| (10⁹/l) | | | | | | | | |
| LYM | 5.2 | 6.3 | 6.6 | 7.0 | 5.3 | 4.6 | 4.4 | 5.7 |
| (10⁹/l) | | | | | | | | |
| MONO | 0.13 | 0.17 | 0.17 | 0.20 | 0.15 | 0.13 | 0.10 | 0.17 |
| (10⁹/l) | | | | | | | | |
| GRAN | 1.10 | 1.17 | 1.30 | 1.40 | 0.85 | 0.77 | 0.77 | 1.23 |
| (10⁹/l) | | | | | | | | |
| HGB | 14.9 | 14.4 | 15.2 | 15.4 | 14.2 | 15.3 | 14.4 | 14.4 |
| (g/L) | | | | | | | | |
| MCH | 19.8 | 19.7 | 20.1 | 19.6 | 19.4 | 18.9 | 19.7 | 19.1 |
| (pg) | | | | | | | | |
| MCHC | 34.6 | 35.4 | 35.2 | 35.4 | 34.9 | 35.6 | 35.4 | 35.7 |
| (g/l) | | | | | | | | |
| RBC | 7.58 | 7.31 | 7.57 | 7.87 | 7.31 | 8.08 | 7.36 | 7.55 |
| (10¹²/1) | | | | | | | | |
| MCV | 57.1 | 55.5 | 57.1 | 55.4 | 55.7 | 53.3 | 55.5 | 53.5 |
| (fl) | | | | | | | | |
| HCT | 43.2 | 40.6 | 43.2 | 43.6 | 40.6 | 43.0 | 40.8 | 40.5 |
| (%) | | | | | | | | |
| RDW % | 14.4 | 14.8 | 14.9 | 15.1 | 14.1 | 14.6 | 14.1 | 14.2 |
| (%) | | | | | | | | |
| PLT | 806 | 991 | 981 | 1115 | 1005 | 933 | 1106 | 1027 |
| (10⁹/l) | | | | | | | | |

As compared to corresponding control group (Group 1), no alterations in plasma chemistry variables were noted in males nor females of the groups treated with any of the target compounds, at any dose levels (Tables 15-18).

TABLE 15
The plasma chemistry parameters of the control group
1 and the groups treated with 30-300 mg/kg of SET135.

| | Sex/Group | | | | | | | |
|---|---|---|---|---|---|---|---|---|
| | M/1 | M/2 | M/3 | M/4 | F/1 | F/2 | F/3 | F/4 |
| ALB | 49 | 50 | 50 | 50 | 58 | 56 | 59 | 59 |
| (g/L) | | | | | | | | |
| ALP | 239 | 234 | 259 | 275 | 179 | 117 | 126 | 156 |
| (U/L) | | | | | | | | |
| ALT | 30 | 33 | 25 | 28 | 22 | 23 | 23 | 20 |
| (U/L) | | | | | | | | |
| AMY | 766 | 757 | 770 | 835 | 614 | 564 | 589 | 590 |
| (U/L) | | | | | | | | |
| TBIL | 4.8 | 5.0 | 4.7 | 5.0 | 4.3 | 5.3 | 5.0 | 5.3 |
| (μmol/L) | | | | | | | | |
| BUN | 6.6 | 5.6 | 5.6 | 5.7 | 6.4 | 6.0 | 5.7 | 5.3 |
| (mmol/L) | | | | | | | | |
| CA | 2.97 | 3.01 | 2.95 | 2.98 | 2.93 | 2.91 | 2.96 | 3.00 |
| (mmol/L) | | | | | | | | |
| PHOS | 2.38 | 2.20 | 2.51 | 2.64 | 2.04 | 2.30 | 2.34 | 2.91 |
| (mmol/L) | | | | | | | | |
| CRE | 37 | 39 | 36 | 38 | 48 | 31 | 44 | 41 |
| (μmol/L) | | | | | | | | |
| GLU | 7.9 | 8.5 | 8.9 | 9.0 | 7.9 | 8.1 | 7.9 | 7.8 |
| (mmol/L) | | | | | | | | |
| NA+ | 141 | 140 | 138 | 140 | 141 | 141 | 140 | 142 |
| (mmol/L) | | | | | | | | |
| K+ | 4.60 | 4.80 | 4.83 | 5.2 | 4.6 | 4.6 | 4.4 | 4.6 |
| (mmol/L) | | | | | | | | |
| TP | 60 | 65 | 61 | 63 | 66 | 64 | 71 | 67 |
| (g/L) | | | | | | | | |
| GLOB | 11 | 15 | 12 | 13 | 8 | 8 | 11 | 9 |
| (g/L) | | | | | | | | |

TABLE 16
The plasma chemistry parameters of the control group
1 and the groups treated with 30-300 mg/kg of SET152.

| | Sex/Group | | | | | | | |
|---|---|---|---|---|---|---|---|---|
| | M/1 | M/5 | M/6 | M/7 | F/1 | F/5 | F/6 | F/7 |
| ALB | 49 | 50 | 51 | 49 | 58 | 56 | 58 | 56 |
| (g/L) | | | | | | | | |
| ALP | 239 | 272 | 267 | 244 | 179 | 84 | 162 | 134 |
| (U/L) | | | | | | | | |
| ALT | 30 | 30 | 25 | 28 | 22 | 23 | 20 | 21 |
| (U/L) | | | | | | | | |
| AMY | 766 | 837 | 838 | 854 | 614 | 548 | 573 | 539 |
| (U/L) | | | | | | | | |
| TBIL | 4.8 | 4.0 | 5.0 | 4.7 | 4.3 | 4.0 | 4.7 | 4.7 |
| (μmol/L) | | | | | | | | |
| BUN | 6.6 | 5.7 | 5.7 | 5.5 | 6.4 | 4.9 | 6.0 | 5.2 |
| (mmol/L) | | | | | | | | |
| CA | 2.97 | 2.99 | 2.98 | 3.04 | 2.93 | 2.90 | 2.87 | 2.92 |
| (mmol/L) | | | | | | | | |
| PHOS | 2.38 | 2.11 | 2.57 | 2.47 | 2.04 | 2.28 | 2.38 | 2.39 |
| (mmol/L) | | | | | | | | |
| CRE | 37 | 42 | 42 | 51 | 48 | 44 | 48 | 51 |
| (μmol/L) | | | | | | | | |
| GLU | 7.9 | 8.0 | 8.2 | 8.6 | 7.9 | 8.0 | 7.8 | 7.7 |
| (mmol/L) | | | | | | | | |
| NA+ | 141 | 140 | 141 | 143 | 141 | 148 | 139 | 140 |
| (mmol/L) | | | | | | | | |
| K+ | 4.60 | 4.6 | 4.7 | 4.5 | 4.6 | 2.9 | 4.5 | 4.8 |
| (mmol/L) | | | | | | | | |
| TP | 60 | 63 | 63 | 63 | 66 | 64 | 66 | 64 |
| (g/L) | | | | | | | | |
| GLOB | 11 | 13 | 12 | 14 | 8 | 7 | 8 | 8 |
| (g/L) | | | | | | | | |

TABLE 17
The plasma chemistry parameters of the control group
1 and the groups treated with 30-300 mg/kg of SET159.

| | Sex/Group | | | | | | | |
|---|---|---|---|---|---|---|---|---|
| | M/1 | M/8 | M/9 | M/10 | F/1 | F/8 | F/9 | F/10 |
| ALB | 49 | 49 | 47 | 50 | 58 | 56 | 57 | 53 |
| (g/L) | | | | | | | | |
| ALP | 239 | 274 | 324 | 284 | 179 | 179 | 135 | 175 |
| (U/L) | | | | | | | | |
| ALT | 30 | 25 | 30 | 24 | 22 | 22 | 21 | 23 |
| (U/L) | | | | | | | | |
| AMY | 766 | 719 | 823 | 806 | 614 | 541 | 542 | 481 |
| (U/L) | | | | | | | | |
| TBIL | 4.8 | 4.0 | 4.3 | 4.3 | 4.3 | 4.0 | 4.3 | 4.0 |
| (μmol/L) | | | | | | | | |
| BUN | 6.6 | 5.9 | 6.8 | 6.1 | 6.4 | 6.2 | 5.1 | 4.8 |
| (mmol/L) | | | | | | | | |
| CA | 2.97 | 2.94 | 2.99 | 3.02 | 2.93 | 2.76 | 2.88 | 2.89 |
| (mmol/L) | | | | | | | | |
| PHOS | 2.38 | 2.44 | 2.43 | 2.42 | 2.04 | 2.59 | 2.65 | 2.80 |
| (mmol/L) | | | | | | | | |
| CRE | 37 | 29 | 36 | 47 | 48 | 39 | 46 | 40 |
| (μmol/L) | | | | | | | | |
| GLU | 7.9 | 8.0 | 8.8 | 7.9 | 7.9 | 7.8 | 7.0 | 7.8 |
| (mmol/L) | | | | | | | | |
| NA+ | 141 | 141 | 141 | 142 | 141 | 139 | 139 | 139 |
| (mmol/L) | | | | | | | | |
| K+ | 4.6 | 5.0 | 5.0 | 4.8 | 4.6 | 4.9 | 4.4 | 4.7 |
| (mmol/L) | | | | | | | | |
| TP | 60 | 60 | 63 | 62 | 66 | 65 | 65 | 62 |
| (g/L) | | | | | | | | |
| GLOB | 11 | 11 | 16 | 13 | 8 | 9 | 8 | 9 |
| (g/L) | | | | | | | | |

TABLE 18
The plasma chemistry parameters of the control group
1 and the groups treated with 30-300 mg/kg of SET171.

| | Sex/Group | | | | | | | |
|---|---|---|---|---|---|---|---|---|
| | M/1 | M/11 | M/12 | M/13 | F/1 | F/11 | F/12 | F/13 |
| ALB | 49 | 51 | 52 | 50 | 58 | 61 | 57 | 56 |
| (g/L) | | | | | | | | |
| ALP | 239 | 260 | 248 | 263 | 179 | 114 | 126 | 145 |
| (U/L) | | | | | | | | |
| ALT | 30 | 26 | 35 | 25 | 22 | 21 | 21 | 21 |
| (U/L) | | | | | | | | |
| AMY | 766 | 810 | 846 | 808 | 614 | 527 | 570 | 550 |
| (U/L) | | | | | | | | |
| TBIL | 4.8 | 4.3 | 4.3 | 5.0 | 4.3 | 5.3 | 5.0 | 4.0 |
| (μmol/L) | | | | | | | | |
| BUN | 6.6 | 5.3 | 6.2 | 6.4 | 6.4 | 6.0 | 6.5 | 6.3 |
| (mmol/L) | | | | | | | | |
| CA | 2.97 | 2.97 | 3.04 | 2.96 | 2.93 | 2.90 | 2.92 | 2.89 |
| (mmol/L) | | | | | | | | |
| PHOS | 2.38 | 2.83 | 3.04 | 2.94 | 2.04 | 2.33 | 2.42 | 2.64 |
| (mmol/L) | | | | | | | | |
| CRE | 37 | 47 | 35 | 29 | 48 | 45 | 38 | 39 |
| (μmol/L) | | | | | | | | |
| GLU | 7.9 | 7.8 | 7.7 | 8.4 | 7.9 | 7.8 | 7.7 | 7.9 |
| (mmol/L) | | | | | | | | |
| NA+ | 141 | 140 | 140 | 141 | 141 | 138 | 137 | 139 |
| (mmol/L) | | | | | | | | |
| K+ | 4.6 | 4.3 | 4.6 | 4.4 | 4.6 | 4.4 | 4.5 | 4.5 |
| (mmol/L) | | | | | | | | |
| TP | 60 | 62 | 64 | 63 | 66 | 70 | 66 | 67 |
| (g/L) | | | | | | | | |
| GLOB | 11 | 12 | 12 | 13 | 8 | 8 | 9 | 11 |
| (g/L) | | | | | | | | |

It can be concluded that 300 mg/kg of oral administration of SET-135, SET-152, SET-159, SET-171 is well tolerated with regards to adverse clinical signs, body weight and clinical pathology parameters.

### REFERENCES

[1] Gluchowski, N. L., Becuwe, M., Walther, T. C., and Farese, R. V., Jr. (2017). Lipid droplets and liver disease: from basic biology to clinical implications. Nat Rev Gastroenterol Hepatol 14, 343-355.
[2] Lee, S., Zhang, C., Liu, Z., Klevstig, M., Mukhopadhyay, B., Bergentall, M., Cinar, R., Stahlman, M., Sikanic, N., Park, J. K., et al. (2017). Network analyses identify liver-specific targets for treating liver diseases. Mol Syst Biol 13, 938.
[3] Chella Krishnan, K., Kurt, Z., Barrere-Cain, R., Sabir, S., Das, A., Floyd, R., Vergnes, L., Zhao, Y., Che, N., Charugundla, S., et al. (2018). Integration of Multi-omics Data from Mouse Diversity Panel Highlights Mitochondrial Dysfunction in Non-alcoholic Fatty Liver Disease. Cell Syst 6, 103-115 e107.
[4] Liu, Z., Zhang, C., Lee, S., Kim, W., Klevstig, M., Harzandi, A. M., Sikanic, N., Arif, M., Stahlman, M., Nielsen, J., et al. (2019). Pyruvate kinase L/R is a regulator of lipid metabolism and mitochondrial function. Metab Eng 52, 263-272.
[5] Zhang, C., Shi, M., Kim, W., Arif, M., Klevstig, M., Li, X., . . . & Mardinoglu, A. (2022). Discovery of therapeutic agents targeting PKLR for NAFLD using drug repositioning. EBioMedicine, 83, 104214.
[6] Godoy-Matos A F, Silva Jnnior W S, Valerio C M. NAFLD as a continuum: from obesity to metabolic syndrome and diabetes. Diabetology & metabolic syndrome, 12.1 (2020): 1-20.
[7] Kim W, Li M, Jin H, Yang H, Turkez H, Uhlen M, et al. Characterization of an in vitro Steatosis Model for Modelling the Activated de novo Lipogenesis in MAFLD Patients. (2023)
[8] Siriwardana R C, Niriella M A, Dassanayake A S, Liyanage C, Gunathilaka B, Jayathunge S, et al. Clinical characteristics and outcome of hepatocellular carcinoma in alcohol related and cryptogenic cirrhosis: a prospective study. Hepatobiliary & Pancreatic Diseases International, 14.4 (2015):401-5.
[9] Ozcan M, Altay O, Lam S, Turkez H, Aksoy Y, Nielsen J, et al. Improvement in the current therapies for hepatocellular carcinoma using a systems medicine approach. Advanced biosystems, 4.6 (2020):2000030.
[10] Siegel R L, Miller K D, Wagle N S, Jemal A. Cancer statistics, Ca Cancer J Clin, 73.1 (2023):17-48.
[11] Marin, J. J. G. et al. Molecular Bases of Drug Resistance in Hepatocellular Carcinoma. Cancers 12, 1663 (2020).
[12] SOLÓRZANO-MELENDEZ, Alejandro, et al. Micronucleated erythrocytes in peripheral blood from neonate rats fed by nursing mothers exposed to X-rays. Environmental and Molecular Mutagenesis, 2021, 62.3: 177-184.
[13] Molecular Operating Environment (MOE), 2019.01; Chemical Computing Group ULC, 1010 Sherbooke St. West, Suite #910, Montreal, QC, Canada, H3A 2R7, 2019.” 2019.
[14] Angell R M, Atkinson F L, Brown M J, Chuang T T, Christopher J A, Cichy-Knight M, Dunn A K, Hightower K E, Malkakorpi S, Musgrave J R, Neu M. N-(3-Cyano-4, 5, 6, 7-tetrahydro-1-benzothien-2-yl) amides as potent, selective, inhibitors of JNK2 and JNK3. Bioorganic & medicinal chemistry letters. 2007 Mar. 1; 17(5):1296-301.
[15] Halgren, Thomas A. 1996. “Merck Molecular Force Field. I. Basis, Form, Scope, Parameterization, and Performance of MMFF94.” Journal of Computational Chemistry 17 (5-6): 490-519.
[16] Bray, N., Pimentel, H., Melsted, P. et al. Near-optimal probabilistic RNA-seq quantification. Nat Biotechnol 34, 525-527 (2016). https://doi.org/10.1038/nbt.3519
[17] Soneson C, Love M I, Robinson M D. Differential analyses for RNA-seq: transcript-level estimates improve gene-level inferences. F1000Res. 2015 Dec. 30; 4:1521.
[18] Love, M. I., Huber, W. & Anders, S. Moderated estimation of fold change and dispersion for RNA-seq data with DESeq2. Genome Biol 15, 550 (2014). https://doi.org/10.1186/s13059-014-0550-8
[19] Stacklies W, Redestig H, Scholz M, Walther D, Selbig J. pcaMethods—a bioconductor package providing PCA methods for incomplete data. Bioinformatics. 2007 May 1; 23(9):1164-7.

Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proc Natl Acad Sci USA. 2005 Oct. 25; 102(43):15545-50.
[21] Liberzon A, Birger C, Thorvaldsdöttir H, Ghandi M, Mesirov J P, Tamayo P. The Molecular Signatures Database (MSigDB) hallmark gene set collection. Cell Syst. 2015 Dec. 23; 1(6):417-425.
[22] Kanehisa M, Furumichi M, Sato Y, Kawashima M, Ishiguro-Watanabe M. KEGG for taxonomy-based analysis of pathways and genomes. Nucleic Acids Res. 2023 Jan. 6; 51(D1):D587-D592.
[23] WU, Tianzhi, et al. clusterProfiler 4.0: A universal enrichment tool for interpreting omics data. The innovation, 2021, 2.3.
[24] Guangchuang Yu, Li-Gen Wang, Yanyan Han, and Qing-Yu He.clusterProfiler: an R Package for Comparing Biological Themes Among Gene Clusters.OMICS: A Journal of Integrative Biology. May 2012.284-287.
[25] Schubert, M., Klinger, B., Klünemann, M. et al. Perturbation-response genes reveal signaling footprints in cancer gene expression. Nat Commun 9, 20 (2018).
[26] Badia-I-Mompel P, Vélez Santiago J, Braunger J, Geiss C, Dimitrov D, Müller-Dott S, Taus P, Dugourd A, Holland C H, Ramirez Flores R O, Saez-Rodriguez J. decoupleR: ensemble of computational methods to infer biological activities from omics data. Bioinform Adv. 2022 Mar. 8; 2(1):vbaco16.
[27] Yu G, Li F, Qin Y, Bo X, Wu Y, Wang S. GOSemSim: an R package for measuring semantic similarity among GO terms and gene products. Bioinformatics. 2010 Apr. 1; 26(7):976-8.
[28] Liao, X. et al. Open MoA: Revealing the Mechanism of Action (MoA) based on Network Topology and Hierarchy. Bioinformatics btad666 (2023)
[29] Eferl, R. et al. Liver Tumor Development: c-Jun Antagonizes the Proapoptotic Activity of p53.
[30] Kim, W. et al. Characterization of an in vitro steatosis model simulating activated de novo lipogenesis in MAFLD patients. iScience 26, 107727 (2023).
[31] Chiyo, T. et al. Galectin-9 Induces Mitochondria-Mediated Apoptosis of Esophageal Cancer In Vitro and In Vivo in a Xenograft Mouse Model. Int. J. Mol. Sci. 20, 2634 (2019).
[32] Jang, W. Y., Lee, J. Y., Lee, S. T., Jun, D. Y. & Kim, Y. H. Inhibition of JNK2 and JNK3 by JNK inhibitor IX induces prometaphase arrest-dependent apoptotic cell death in human Jurkat T cells. Biochem. Biophys. Res. Commun. 452, 845-851 (2014).
[33] Shi, J. et al. JNK inhibitor IX restrains pancreatic cancer through p53 and p21. Front. Oncol. 12, 1006131 (2022).
[34] Ho, J. S. L., Ma, W., Mao, D. Y. L. & Benchimol, S. p53-Dependent Transcriptional Repression of c-myc Is Required for G₁ Cell Cycle Arrest. Mol. Cell. Biol. 25, 7423-7431 (2005).
[35] Jindal, A. et al. Milciclib and sorafenib synergistically downregulate c-Myc to suppress tumor growth in an orthotopic murine model of human hepatocellular carcinoma. J. Transl. Sci. 7, (2021).
[36] Cai, M.-H. et al. Regorafenib suppresses colon tumorigenesis and the generation of drug resistant cancer stem-like cells via modulation of miR-34a associated signaling. J. Exp. Clin. Cancer Res. 37, 151 (2018).
[37] Sha, B. et al. USP8 inhibitor-induced DNA damage activates cell cycle arrest, apoptosis, and autophagy in esophageal squamous cell carcinoma. Cell Biol. Toxicol. 39, 2011-2032 (2023).
[38] Ozaki, T. & Nakagawara, A. Role of p53 in Cell Death and Human Cancers. Cancers 3, 994-1013 (2011).
[39] Shen, Y. & White, E. p53-Dependent apoptosis pathways. in Advances in Cancer Research vol. 82 55-84 (Elsevier, 2001).
[40] Mrakovcic, M. & Fröhlich, L. p53-Mediated Molecular Control of Autophagy in Tumor Cells. Biomolecules 8, 14 (2018).
[41] Lei, K. & Davis, R. J. JNK phosphorylation of Bim-related members of the Bcl2 family induces Bax-dependent apoptosis. Proc. Natl. Acad. Sci. 100, 2432-2437 (2003).
[42] Zhang, S. et al. Inhibition of the PINK1-Parkin Pathway Enhances the Lethality of Sorafenib and Regorafenib in Hepatocellular Carcinoma. Front. Pharmacol. 13, 851832 (2022).
[43] Wei, Y., Sinha, S. C. & Levine, B. Dual Role of JNK1-mediated phosphorylation of BCl-2 in autophagy and apoptosis regulation. Autophagy 4, 949-951 (2008).
[44] Wei, Y., Pattingre, S., Sinha, S., Bassik, M. & Levine, B. JNK1-Mediated Phosphorylation of BCl-2 Regulates Starvation-Induced Autophagy. Mol. Cell 30, 678-688 (2008).
[45] Lin, C.-Y. et al. Simultaneous induction of apoptosis and necroptosis by Tanshinone IIA in human hepatocellular carcinoma HepG2 cells. Cell Death Discov. 2, 16065 (2016).
[46] Dhuriya, Y. K. & Sharma, D. Necroptosis: a regulated inflammatory mode of cell death. J. Neuroinflammation 15, 199 (2018).
[47] Galluzzi, L., Kepp, O., Chan, F. K.-M. & Kroemer, G. Necroptosis: Mechanisms and Relevance to Disease. Annu. Rev. Pathol. Mech. Dis. 12, 103-130 (2017).
[48] Goodall, M. L. et al. The Autophagy Machinery Controls Cell Death Switching between Apoptosis and Necroptosis. Dev. Cell 37, 337-349 (2016).
[49] Ichimura, Y. & Komatsu, M. Activation of p62/SQSTM1-Keap1—Nuclear Factor Erythroid 2-Related Factor 2 Pathway in Cancer. Front. Oncol. 8, 210 (2018).
[50] Somlapura, M. et al. Different Roles of p62 (SQSTM1) Isoforms in Keratin-Related Protein Aggregation. Int. J. Mol. Sci. 22, 6227 (2021).
[51] Rodriguez-Vargas, J. M. et al. ROS-induced DNA damage and PARP-1 are required for optimal induction of starvation-induced autophagy. Cell Res. 22, 1181-1198 (2012).
[52] Filomeni, G., De Zio, D. & Cecconi, F. Oxidative stress and autophagy: the clash between damage and metabolic needs. Cell Death Differ. 22, 377-388 (2015).
[53] Środa-Pomianek, K. et al. Increased lipid peroxidation, apoptosis and selective cytotoxicity in colon cancer cell line LoVo and its doxorubicin-resistant subline LoVo/Dx in the presence of newly synthesized phenothiazine derivatives. Biomed. Pharmacother. 106, 624-636 (2018).
[54] Anuradha, C. D., Kanno, S. & Hirano, S. Oxidative damage to mitochondria is a preliminary step to caspase-3 activation in fluoride-induced apoptosis in HL-60 cells. Free Radic. Biol. Med. 31, 367-373 (2001).
[55] Yang, W. S. et al. Regulation of Ferroptotic Cancer Cell Death by GPX4. Cell 156, 317-331 (2014).
[56] Gong, Y. et al. The role of necroptosis in cancer biology and therapy. Mol. Cancer 18, 100 (2019).
[57] Jordan, A., Hadfield, J. A., Lawrence, N. J. & McGown, A. T. Tubulin as a target for anticancer drugs: Agents which interact with the mitotic spindle. Med. Res. Rev. 18, 259-296 (1998).
[58] Khwaja, S., Kumar, K., Das, R. & Negi, A. S. Microtubule associated proteins as targets for anticancer drug development. Bioorganic Chem. 116, 105320 (2021).
[59] Lee, C.-C., Cheng, Y.-C., Chang, C.-Y., Lin, C.-M. & Chang, J.-Y. Alpha-tubulin acetyltransferase/MEC-17 regulates cancer cell migration and invasion through epithelial-mesenchymal transition suppression and cell polarity disruption. Sci. Rep. 8, 17477 (2018).
[60] Izdebska, M., Zielinska, W., Grzanka, D. & Gagat, M. The Role of Actin Dynamics and Actin-Binding Proteins Expression in Epithelial-to-Mesenchymal Transition and Its Association with Cancer Progression and Evaluation of Possible Therapeutic Targets. BioMed Res. Int. 2018, 1-13 (2018).
[61] Simiczyjew, A., Mazur, A. J., Dratkiewicz, E. & Nowak, D. Involvement of β- and γ-actin isoforms in actin cytoskeleton organization and migration abilities of bleb-forming human colon cancer cells. PLOS ONE 12, e0173709 (2017).
[62] Yu, G. et al. GOSemSim: an R package for measuring semantic similarity among GO terms and gene products. Bioinformatics 26, 976-978 (2010).
[63] Subramanian, A. et al. Gene set enrichment analysis: A knowledge-based approach for interpreting genome-wide expression profiles. Proc. Natl. Acad. Sci. 102, 15545-15550 (2005).
[64] Kanehisa, M., Furumichi, M., Sato, Y., Kawashima, M. & Ishiguro-Watanabe, M. KEGG for taxonomy-based analysis of pathways and genomes. Nucleic Acids Res. 51, D587-D592 (2023).
[65] Schubert, M. et al. Perturbation-response genes reveal signaling footprints in cancer gene expression. Nat. Commun. 9, 20 (2018).

1. A compound of formula (I) 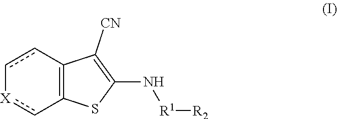
or a pharmaceutically acceptable salt thereof, wherein:
both [symbol] are single bonds or double bonds;
X is selected from —(CH₂)₂— and —NR₃— when [symbol] is a single bond;
X is —CH— when [symbol] is a double bond;
R1 is independently selected from a carbonyl and a sulphonyl;
R2 is selected from the group consisting of 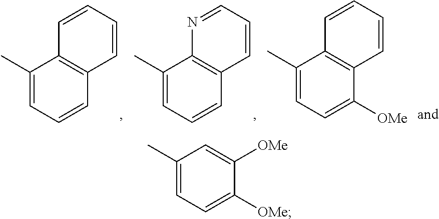
R3 is selected from the group consisting of —H, 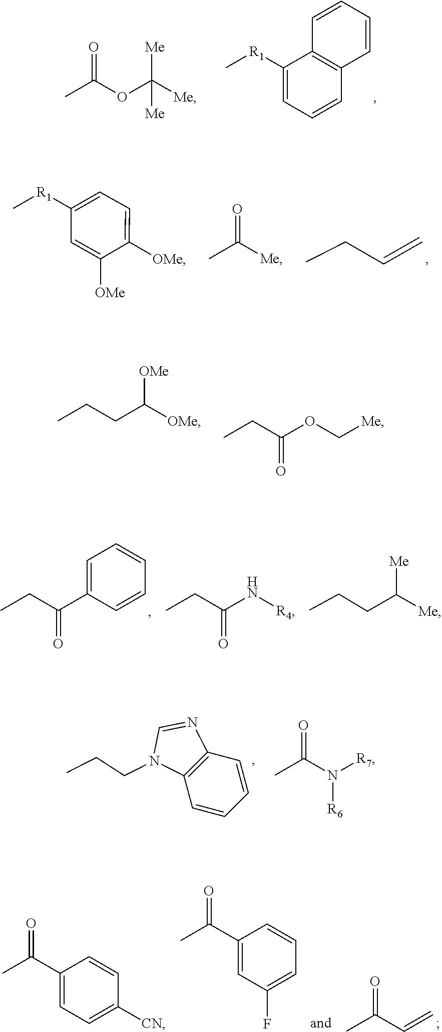
R4 is 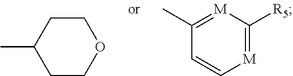
M is N or CH;
R5 is —H, halide, 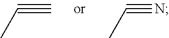
R6 is —H, -Me or phenyl; and
R7 is C1-C3 alkyl or an optionally substituted aryl;
provided that it is not a compound according to any one of formulae (II)-(V).

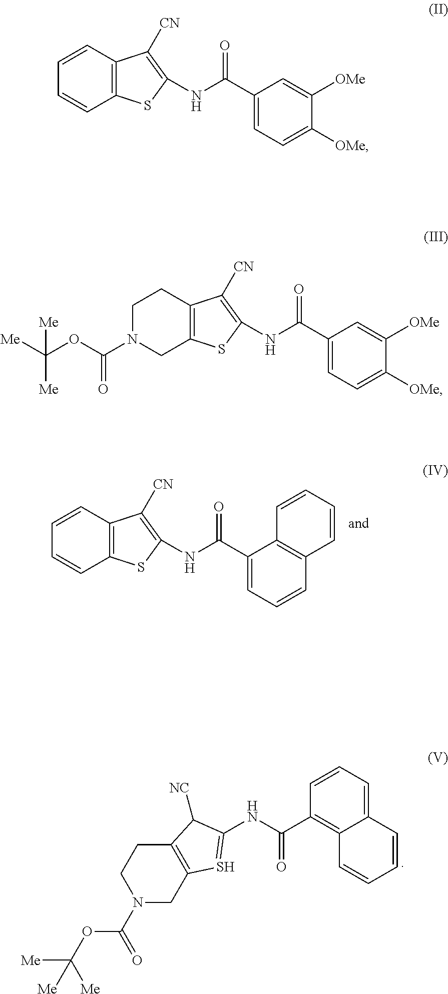

2. The compound of claim 1, wherein R4 is selected from the group consisting of

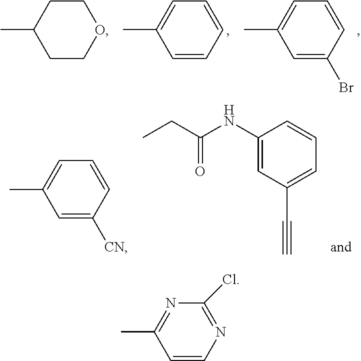

3. The compound of claim 1, wherein 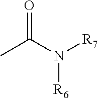
is selected from the group consisting of 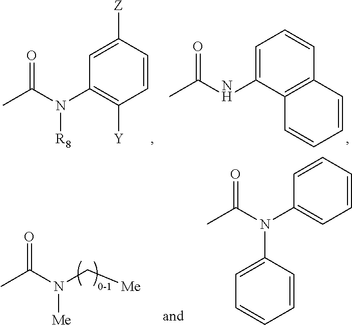
wherein:
Z is selected from the group consisting of —H, -Me and —OMe;
Y is selected from the group consisting of —H, —F, —Br, —Cl, —OMe, —OCF₃, —CF₃, -Me and NO₂;
and
R8 is selected from —H and -Me.

4. The compound of claim 3 wherein Y is selected from the group consisting of —H, —F, —Br, —Cl, —OMe, -Me and —NO₂.

5. The compound of claim 1, wherein R1 is a carbonyl.

6. The compound of claim 1, wherein [symbol] is a single bond and X is —NR₃—.

7. The compound of claim 1, wherein R2 is

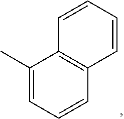

8. The compound of claim 1 having the formula (VI)

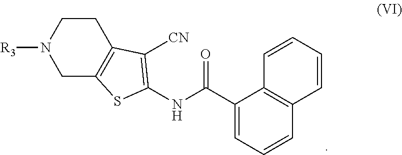

9. The compound of claim 8 wherein R3 is selected from 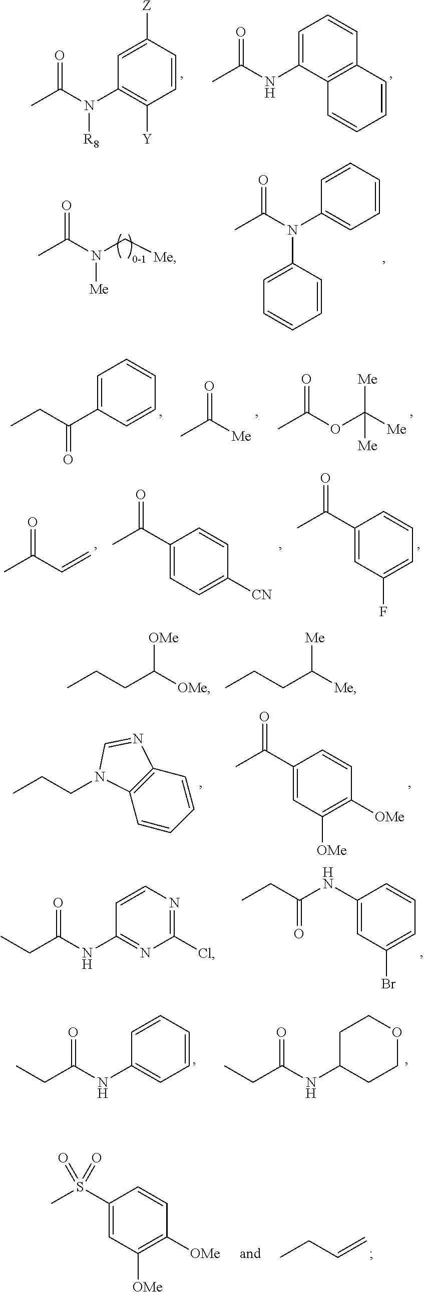
and wherein:
Z is —H, Y is —F, —Br, —Cl, —OMe or —NO₂, and R8 is —H;
Z is —OMe, Y is —H, and R8 is —H;
Z is -Me, Y is —F, and R8 is —H; or
Z is —H, Y is —H, and R8 is -Me.

10. The compound of claim 9 wherein R3 is selected from

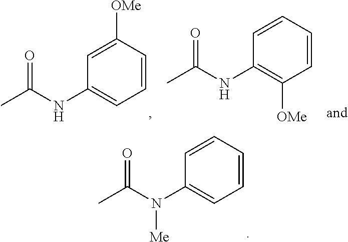

11. The compound according to claim 1, wherein X is —(CH₂)₂— and R2 is

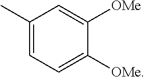

12. (canceled)

13. A pharmaceutical composition comprising a compound according to claim 1.

14. A method of treating fatty liver disease or hepatocellular carcinoma (HCC) comprising administering to a subject in need thereof a compound according to formulae (V) or (VII)

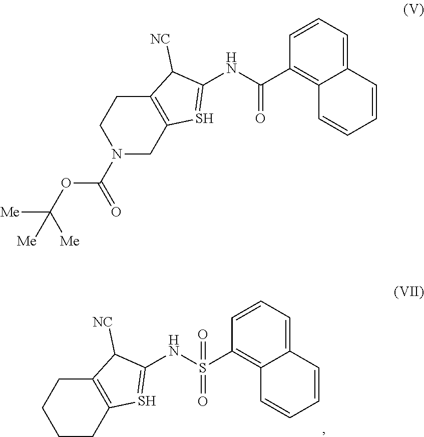

15. The method according to claim 14, wherein the method comprises oral administration of the compound.

16. The method according to claim 14, wherein said fatty liver disease is non-alcoholic fatty liver disease (NAFLD), wherein said NAFLD optionally has progressed to non-alcoholic steatohepatitis (NASH).

17. The method according to claim 14, wherein the method further comprises detection of overexpression of PKLR in the liver of the subject to be treated prior to administration of the compound to the subject.

18. A method of treating fatty liver disease or hepatocellular carcinoma (HCC) comprising administering to a subject in need thereof the pharmaceutical composition according to claim 13.

19. The method according to claim 18, wherein the method comprises oral administration of the compound.

20. The method according to claim 18, wherein said fatty liver disease is non-alcoholic fatty liver disease (NAFLD), wherein said NAFLD optionally has progressed to non-alcoholic steatohepatitis (NASH).

21. The method according to claim 18, wherein the method further comprises detection of overexpression of PKLR in the liver of the subject to be treated prior to administration of the compound to the subject.
